(19) United States
(12) Patent Application Publication
(10) Pub. No.: US 2024/0125768 A1
(43) Pub. Date: Apr. 18, 2024

(54) METHODS FOR MODULATING A SRC-1 CONDENSATE

(71) Applicant: ETERN BIOPHARMA (SHANGHAI) CO., LTD., Shanghai (CN)

(72) Inventors: Guangya ZHU, Shanghai (CN); Jingjing XIE, Shanghai (CN); Jidong ZHU, Shanghai (CN); Xin GUO, Shanghai (CN); Hao HE, Shanghai (CN)

(73) Assignee: ETERN BIOPHARMA ( SHANGHAI)CO, LTD., Shanghai (CN)

(21) Appl. No.: 18/264,764

(22) PCT Filed: Feb. 9, 2022
(86) PCT No.: PCT/CN2022/075698

§ 371 Date: Aug. 9, 2023

(30) Foreign Application Priority Data
Feb. 10, 2021 (WO) PCT/CN2021/076538

(51) Int. Cl. G01N 33/50; A61K 31/47; A61P 35/00; C07K 14/47; C12N 9/12; C12N 15/113; G01N 33/573

(52) U.S. Cl. CPC G01N 33/5017; A61K 31/47; A61P 35/00; C07K 14/4705; C12N 9/1247; C12N 15/1137; G01N 33/573; G01N 2333/4706; G01N 2333/91057; G01N 2333/91255

(57) ABSTRACT

Provided are methods for modulating a SRC-1 condensate to regulate transcription of one or more genes, methods of treating diseases and conditions using SRC-1 inhibitors, and methods for screening of agents that modulate SRC-1 condensate.

### FIELD OF THE INVENTION

The present disclosure generally relates to methods for modulating a SRC-1 condensate to regulate transcription of one or more genes, and methods for screening of agents that modulate SRC-1 condensate.

### BACKGROUND

Yes-associated protein (YAP) is a transcriptional coactivator that plays essential role in promoting cell proliferation, development and stem-cell fate (Meng, Z., et al., Genes. Dev. 30, 1-17 (2016)). Aberrant YAP activation is prevalent in diverse types of human solid cancers (Harvey, K. F., et al., Nat. Rev. Cancer 13, 246-257 (2013)). In mammals, a kinase cascade including MST1/2 and LATS1/2 phosphorylate YAP to prevent its nuclear translocation and subsequent association with the TEA-domain transcription factors TEAD1-4 in the canonical Hippo pathway. Previous studies have intensively emphasized on the upstream signals from the Hippo kinase cascade that regulate YAP (Halder, G., et al., Nat. Rev. Mol. Cell Biol. 13, 591-600 (2012)), however, the epigenetic regulatory mechanism of YAP transcriptional activity is far less understood.

The p160 family of steroid receptor coactivator SRC-1 function as transcriptional coactivator for nuclear hormone receptors, as well as many other transcription factors(Onate, S. A., et al., Science 270, 1354-1357 (1995), Lonard, D. M. & O'Malley, B. W. Mol. Cell 27, 691-700 (2007), York, B. & O'Malley, B. W. J. Biol. Chem. 285, 38743-38750 (2010)). Mounting evidence revealed that gene regulation occurs in transcriptional condensates, which concentrate transcription factors, coactivators, the transcription and elongation machinery for spatial and temporal transcription control (Hnisz, D., et al., Cell 169, 13-23 (2017), Alberti, S., et al., Cell 176, 419-434 (2019), Lee, T. I. & Young, R. A. Cell 152, 1237-1251 (2013)). Therefore, there is a need for exploration of the roles of YAP/TEAD and SRC-1 in transcription regulation for develop new therapeutic strategies for the treatment of cancer.

### BRIEF SUMMARY OF THE INVENTION

Throughout the present disclosure, the articles “a,” “an,” and “the” are used herein to refer to one or to more than one (i.e., to at least one) of the grammatical object of the article. By way of example, “an antibody” means one antibody or more than one antibody.

In one aspect, the present disclosure provides a method of modulating transcription of one or more genes in a cell or in a subject, comprising modulating a transcriptional SRC-1 condensate comprising at least SRC-1, wherein the transcriptional SRC-1 condensate regulates transcription of the one or more genes.

In some embodiments, the transcriptional SRC-1 condensate further comprises a first component capable of interacting with SRC-1.

In some embodiments, the first component comprises Yes-associated protein (YAP), Estrogen receptor (ER), androgen receptor (AR), vitamin D receptor(VDR) and AP-1.

In some embodiments, the transcriptional SRC-1 condensate further comprises a second component interacting with the first component.

In some embodiments, the second component comprises a TEA-domain transcription factor.

In some embodiments, the TEA-domain transcription factor comprises TEAD1, TEAD2, TEAD3, TEAD4 or any combination thereof.

In some embodiments, the transcriptional SRC-1 condensate further comprises a RNA polymerase.

In some embodiments, the transcriptional SRC-1 condensate is modulated by modulating or reducing formation, composition, stability, and/or activity of the transcriptional SRC-1 condensate.

In some embodiments, the transcriptional SRC-1 condensate is modulated by contacting with a SRC-1 condensate inhibitor. In certain embodiments, the SRC-1 condensate inhibitor reduces the formation, composition, stability, or activity of the transcriptional SRC-1 condensate.

In some embodiments, the SRC-1 condensate inhibitor is capable of
a) reducing formation or stability of a SRC-1 condensate,
b) reducing or eliminating interactions between SRC-1 and one or more components in the transcriptional condensate, optionally in a SRC-1 selective manner,
c) reducing or eliminating binding of SRC-1 to one or more components in the transcriptional condensate, optionally in a SRC-1 selective manner, or
d) sequestering SRC-1 outside the transcriptional condensate.

In some embodiments, the one or more components comprises YAP, Estrogen receptor (ER), androgen receptor (AR), vitamin D receptor (VDR) and AP-1.

In some embodiments, the SRC-1 condensate inhibitor interacts with an intrinsic disorder domain of SRC-1.

In some embodiments, the SRC-1 condensate inhibitor binds to a non-IDD region of SRC-1, and optionally allosterically induces a conformational change in the IDD.

In some embodiments, the SRC-1 condensate inhibitor sequesters SRC-1 outside the transcriptional condensate, optionally without significantly dissolving the transcriptional condensate without SRC-1.

In some embodiments, the SRC-1 condensate inhibitor comprises a peptide, nucleic acid, or small molecule.

In some embodiments, the SRC-1 condensate inhibitor decreases level of transcriptional SRC-1 condensate by at least 30% (e.g. at least 40%, 50%, 60%, or 70%) at a concentration of no more than 20 uM.

In some embodiments, the SRC-1 condensate inhibitor comprises elvitegravir (EVG), or competes with EVG for binding to SRC-1, or induces a conformational change in SRC-1 at least comparable to that induced by EVG.

In some embodiments, the SRC-1 condensate inhibitor has comparable activity to EVG or higher activity than EVG in decreasing level of transcriptional SRC-1 condensate.

In some embodiments, the transcription of the one or more genes is associated with an oncogenic signaling pathway.

In some embodiments, the one or more genes comprise one or more oncogenes.

In some embodiments, the one or more genes comprise one or more YAP target genes.

In some embodiments, the one or more YAP target genes are selected from the group consisting of ANKRD1, CTGF, and CYR61.

In some embodiments, the cell or the subject has an elevated expression level of SRC-1 relative to a reference level.

In another aspect, the present disclosure provides a method of modulating transcription of one or more YAP target genes in a cell or in a subject, comprising modulating SRC-1 by a SRC-1 inhibitor.

In some embodiments, the SRC-1 inhibitor is capable of reducing expression level or reducing biological activity of SRC-1, or is a SRC-1 condensate inhibitor.

In some embodiments, the SRC-1 inhibitor comprises a peptide, nucleic acid, or small molecule.

In some embodiments, the nucleic acid comprises an oligonucleotide specifically hybridizable to SRC-1 mRNA, or a polynucleotide encoding the oligonucleotide.

In some embodiments, the oligonucleotide comprises siRNA, shRNA, miRNA, or antisense oligonucleotide.

In some embodiments, the SRC-1 inhibitor is a SRC-1 mimetic.

In some embodiments, the one or more YAP target genes are selected from the group consisting of ANKRD1, CTGF, and CYR61.

In some embodiments, the cell or the subject has an elevated expression level of SRC-1 relative to a reference level.

In another aspect, the present disclosure provides a method of treating a SRC-1 condensate associated disease or condition, or YAP-associated disease or condition in a subject, comprising administering to the subject a pharmaceutically effective amount of a SRC-1 inhibitor.

In some embodiments, the disease or condition is characterized in an elevated expression level of SRC-1 relative to a reference level.

In some embodiments, the disease or condition is associated with aberrant expression of an oncogene.

In some embodiments, the disease or condition is associated with aberrant expression of a YAP target gene.

In some embodiments, the disease or condition is associated with aberrant YAP transcription activity.

In some embodiments, the disease or condition is cancer.

In some embodiments, the cancer is metastatic.

In some embodiments, the cancer is breast cancer, lung cancer, adrenal cancer, lymphoepithelial neoplasia, adenoid cell carcinoma, lymphoma, acoustic neuroma, acute lymphocytic leukemia, acral lentiginous melanoma, acute myeloid leukemia, acrospiroma, chronic lymphocytic leukemia, acute eosinophilic leukemia, liver cancer, acute erythrocyte leukemia, small cell lung cancer, acute lymphocytic leukemia, non-small cell lung cancer, acute megakaryoblastic leukemia, MALT lymphoma, acute monocytic leukemia, malignant fibrous histiocytoma, acute promyelocytic leukemia, malignant peripheral schwannomas, mantle cell lymphoma, adenocarcinoma, marginal zone B-cell lymphoma, malignant hippocampal tumor, adenoid cystic carcinoma, gland tumor, adenoma-like odontogenic tumor, mast cell leukemia, adenosquamous carcinoma, mediastinal germ cell tumor, adipose tissue tumor, breast medullary carcinoma, adrenocortical carcinoma, medullary thyroid carcinoma, adult T cell leukemia/lymphoma, Medulloblastoma, invasive NK cell leukemia, melanoma, AIDS-related lymphoma, meningioma, lung rhabdomyosarcoma, Merkel cell carcinoma, alveolar soft tissue sarcoma, mesothelioma, ameloblastoma, metastatic urothelial carcinoma, anaplastic large cell lymphoma, mixed Müllerian tumor, thyroid undifferentiated carcinoma, mucinous neoplasm, angioimmunoblastic T-cell lymphoma, multiple myeloma, angiomyolipoma, muscle tissue tumor, angiosarcoma, mycosis fungoides, astrocytoma, myxoid liposarcoma, atypical deformed rhabdoid tumor, myxoma, B-cell chronic lymphocytic leukemia, mucinous sarcoma, B-cell lymphoblastic leukemia, nasopharyngeal carcinoma, B-cell lymphoma, schwannomas, basal cell carcinoma, neuroblastoma, biliary tract cancer, neurofibromatosis, bladder cancer, neuroma, blastoma, nodular melanoma, bone cancer, eye cancer, Brenner tumor, oligodendroxoma, brown tumor, oligodendroglioma, Burkitt's lymphoma, eosinophilic breast cancer, brain cancer, optic nerve tumor cancer, oral cancer carcinoma in situ, osteosarcoma, carcinosarcoma, ovarian cancer, cartilage tumor, pulmonary sulcus tumor, papillary thyroid carcinoma, myeloma, paraganglioma, chondroma, pineal blastoma, chordoma, pineal cell tumor, choriocarcinoma, pituitary tumor, choroid plexus papilloma, pituitary adenoma, kidney clear cell sarcoma, pituitary tumor, craniopharyngioma, plasmacytoma, cutaneous T-cell lymphoma, multiple embryonic cell tumor, cervical cancer, precursor T lymphoblastic lymphoma, colorectal cancer, primary central nervous system lymphoma, Degos disease, primary effusion lymphoma, proliferative small round cell tumor, primary preformed peritoneal cancer, diffuse large B-cell lymphoma, prostate cancer, embryonic dysplasia of neuroepithelial neoplasia, pancreatic cancer, anaplastic cell tumor, pharyngeal carcinoma, embryonic carcinoma, peritoneal pseudomyxoma, endocrine gland tumor, renal cell carcinoma, enteropathy-associated T-cell lymphoma, endodermal sinus tumor, renal medullary carcinoma, retinoblastoma, esophageal cancer, rhabdomyosarcoma, endadelphos, rhabdomyosarcoma, fibroids, Richter's transformation, fibrosarcoma, rectal cancer, follicular lymphoma, sarcoma, follicular thyroid cancer, schwannoma, ganglion cell tumor, seminoma, gastrointestinal cancer, Sertoli cell tum, germ cell tumor, sex cord-gonadal stromal tumor, pregnancy choriocarcinoma, signet ring cell carcinoma, giant cell fibroblastoma, skin cancer, bone giant cell tumor of bone, small blue round cell tumor, glioma, small cell carcinoma, glioblastoma multiforme, soft tissue sarcoma, glioma, somatostatin tumor, glioma brain, soot wart, pancreatic high glucagonoma, spinal tumor, Gonadoblastoma, spleen marginal lymphoma, granulosa cell tumor, squamous cell carcinoma, estrogen tumor, synovial sarcoma, gallbladder cancer, Sezary disease, gastric cancer, small intestine cancer, hairy cell leukemia, squamous cell carcinoma, hemangioblastoma, gastric cancer, head and neck cancer, T-cell lymphoma, vascular epithelioma, testicular cancer, hematological malignancies, sarcoma, hepatoblastoma, thyroid cancer, hepatosplenic T-cell lymphoma, transitional cell carcinoma, Hodgkin's lymphoma, laryngeal cancer, non-Hodgkin's lymphoma, urachal cancer, invasive lobular carcinoma, genitourinary cancer, intestinal cancer, urothelial carcinoma, renal cancer, uveal melanoma, laryngeal cancer, uterine cancer, malignant freckle-like sputum, verrucous carcinoma, lethal midline granuloma, visual pathway glioma, leukemia, vulvar cancer, testicular stromal tumor, vaginal cancer, liposarcoma, Waldenstrom's macroglobulinemia Disease, adenolymphoma, lymphangioma, nephroblastoma, lymphangisarcoma

In some embodiments, the cancer is breast cancer, lung cancer (optionally non-small cell lung cancer), uveal melanoma, liver cancer, head neck cancer and squamous carcinoma, mesothelioma, or malignant pleural mesothelioma.

In some embodiments, the SRC-1 inhibitor is capable of reducing expression level or reducing biological activity of SRC-1.

In some embodiments, the SRC-1 inhibitor comprises a peptide, nucleic acid, or small molecule.

In some embodiments, the nucleic acid comprises an oligonucleotide specifically hybridizable to SRC-1 mRNA, or a polynucleotide encoding the oligonucleotide.

In some embodiments, the oligonucleotide comprises siRNA, shRNA, miRNA, or antisense oligonucleotide.

In some embodiments, the SRC-1 inhibitor is a SRC-1 mimetic.

In some embodiments, the SRC-1 inhibitor comprises a SRC-1 condensate inhibitor.

In some embodiments, the SRC-1 condensate inhibitor is capable of:
a) reducing formation or stability of a SRC-1 condensate,
b) reducing or eliminating interactions between SRC-1 and one or more components in the transcriptional condensate, optionally in a SRC-1 selective manner,
c) reducing or eliminating binding of SRC-1 to one or more components in the transcriptional condensate, optionally in a SRC-1 selective manner, or
d) sequestering SRC-1 outside the transcriptional condensate.

In some embodiments, the one or more components comprises YAP.

In some embodiments, the SRC-1 condensate inhibitor interacts with an intrinsic disorder domain of SRC-1.

In some embodiments, the SRC-1 condensate inhibitor binds to a non-IDD region of SRC-1, and optionally allosterically induces a conformational change in the IDD.

In some embodiments, the SRC-1 condensate inhibitor sequesters SRC-1 outside the transcriptional condensate, optionally without significantly dissolving the transcriptional condensate without SRC-1.

In some embodiments, the SRC-1 condensate inhibitor decreases level of transcriptional SRC-1 condensate by at least 30% (e.g. at least 40%, 50%, 60%, or 70%) at a concentration of no more than 20 uM.

In some embodiments, the SRC-1 inhibitor comprises elvitegravir (EVG), or competes with EVG for binding to SRC-1, or induces a conformational change in SRC-1 at least comparable to that induced by EVG.

In some embodiments, the SRC-1 condensate inhibitor has comparable activity to EVG or higher activity than EVG in decreasing level of transcriptional SRC-1 condensate.

In another aspect, the present disclosure provides a method of screening for an agent that modulates a SRC-1 condensate, comprising:
a) providing the SRC-1 condensate and assessing one or more physical properties or one or more biological effects of the condensate,
b) contacting the SRC-1 condensate with a test agent, and
c) assessing whether the test agent causes a change in the one or more physical properties or one or more biological effects of the SRC-1 condensate.

In some embodiments, the test agent is identified as modulating the condensate if it causes a change in the one or more physical properties or one or more biological effects of the SRC-1 condensate.

In another aspect, the present disclosure provides a method of identifying an agent that modulates formation of a SRC-1 condensate, comprising:
a. providing components capable of forming the SRC-1 condensate;
b. contacting the components with a test agent under the condition suitable for formation of the SRC-1 condensate, and
c. assessing whether presence of the test agent affects formation of the SRC-1 condensate or one or more biological effects of the SRC-1 condensate.

In some embodiments, the test agent is identified as modulating the formation of the condensate if it affects formation of the condensate or affects the one or more biological effects of the SRC-1 condensate.

In some embodiments, the SRC-1 condensate is an isolated synthetic condensate, or is in the form of an isolated cellular composition comprising the SRC-1 condensate.

In some embodiments, the SRC-1 condensate is inside a cell or inside nucleus.

In some embodiments, the SRC-1 condensate is a transcriptional condensate.

In some embodiments, one or more biological effects of the transcriptional condensate is assessed based on expression of a target gene in a condensate-dependent manner.

In some embodiments, the target gene is a reporter gene.

In some embodiments, the target gene is a YAP regulated gene.

In some embodiments, the transcriptional SRC-1 condensate further comprises a first component capable of interacting with SRC-1.

In some embodiments, the first component comprises Yes-associated protein (YAP), Estrogen receptor(ER), androgen receptor (AR), vitamin D receptor (VDR) and AP-1.

In some embodiments, the transcriptional condensate further comprises a second component interacting with the first component.

In some embodiments, the second component comprises a TEA-domain transcription factor.

In some embodiments, the TEA-domain transcription factor comprises TEAD1, TEAD2, TEAD3, TEAD4 or any combination thereof.

In some embodiments, the transcriptional condensate further comprises a RNA polymerase.

In another aspect, the present disclosure provides a synthetic SRC-1 condensate comprising at least SRC-1 or a fragment thereof comprising an intrinsic disorder domain of SRC-1.

In some embodiments, the fragment is fused with an inducible oligomerization domain.

In another aspect, the present disclosure provides an in vitro screening system comprising SRC-1 or a fragment thereof comprising an intrinsic disorder domain of SRC-1, and a detectable label, wherein the SRC-1 or the fragment thereof is capable of forming SRC-1 condensate.

In some embodiments, the detectable label is attached to the SRC-1 or the fragment thereof.

In some embodiments, the detectable label comprises a fluorophore, a radioisotope, a colorimetric substrate, or an antigenic epitope.

In some embodiments, the in vitro screening system further comprises further comprises a first component capable of interacting with SRC-1.

In some embodiments, the first component comprises Yes-associated protein (YAP), Estrogen receptor(ER), androgen receptor (AR), vitamin D receptor(VDR) and AP-1.

In some embodiments, the in vitro screening system further comprises a second component interacting with the first component.

In some embodiments, the second component comprises a TEA-domain transcription factor.

In some embodiments, the TEA-domain transcription factor comprises TEAD1, TEAD2, TEAD3, TEAD4 or any combination thereof.

In some embodiments, the in vitro screening system further comprises a RNA polymerase.

In some embodiments, the in vitro screening system further comprises a cell lysate or a nuclear lysate.

In another aspect, the present disclosure provides a modified host cell expressing SRC-1 or a fragment thereof comprising an intrinsic disorder domain, wherein the SRC-1 or the fragment is capable of forming SRC-1 condensate, and wherein the host cell further comprises a detectable label that allows for detection of the SRC-1 condensate.

In some embodiments, the detectable label is attached to the SRC-1 or the fragment thereof.

In some embodiments, the detectable label comprises a fluorophore, a radioisotope, a colorimetric substrate, or an antigenic epitope.

In some embodiments, the modified host cell further comprises a YAP-responsive reporter construct.

In some embodiments, the SRC-1-responsive reporter construct comprises a reporter gene operably linked to a promoter responsive to YAP activity.

In some embodiments, the host cell is a tumor cell.

In another aspect, the present disclosure provides a modified host cell expressing: a) SRC-1 or a fragment thereof comprising an intrinsic disorder domain thereof, and b) YAP or a functional equivalent thereof, wherein the host cell comprises a YAP-responsive reporter construct.

In some embodiments, the YAP-responsive reporter construct comprises a reporter gene operably linked to a promoter responsive to YAP activity.

In another aspect, the present disclosure provides a method of screening for an agent that inhibits SRC-1, comprising:
a. contacting a test agent with the modified host cell provided herein under a condition suitable for expression of the reporter gene;
b. assessing change in expression of the reporter gene in response to the test agent; wherein the change in expression of the reporter gene indicates inhibition of SRC-1.

### BRIEF DESCRIPTION OF THE DRAWINGS

The accompanying drawings, which are incorporated herein, form part of the specification. Together with this written description, the drawings further serve to explain the principles of, and to enable a person skilled in the relevant art(s), to make and use the present disclosure.

FIG. 1A illustrates the correlation of YAP mRNA level and YAP copy number in various cancer cell lines analyzed form Cancer Cell Line Encyclopedia (CCLE) database.

FIG. 1B illustrates the protein abundance of YAP and TAZ in indicated cell lines.

FIG. 1C illustrates the qPCR analysis of CTGF in SF268 cells subjected to a genetic screen using siRNA library containing two siRNAs for 15 reported HATs. Error bars show mean±s.e.m. (n=3). *p<0.05. **p<0.01.

FIG. 1D illustrates GSEA enrichment plots using a signature comparing SRC-1 knockdown (siRNApool) to control cells shows negative enrichment for gene set corresponding to YAP target genes.

FIG. 2A illustrates live-cell images showing the colocalization of TEAD4-mTagBFP (blue) condensates, mClover3-YAP (green) condensates and mScarlet-SRC1(red) condensates in nucleus. These three proteins all exhibit discrete puncta distribution in nucleus and have obvious co-localization in puncta. Scale bar represents 5 μm.

FIGS. 2B-D illustrate representative images of FRAP experiments in SF268 cells co-expressed with TEAD4-mTagBFP (blue), mClover3-YAP (green) and mScarlet-SRC-1(red). The arrow indicates the region of photobleaching. After bleaching, the droplet exhibited a crescent shape. With the passage of time, the droplet gradually reorganized and returned to its original state. FIG. 2B illustrate the mScarlet-SRC1 was bleached using a 561-nm laser beam. FIG. 2C illustrate the TEAD4-mTagBFP (blue) was bleached using a 405-nm laser beam. FIG. 2D illustrate the mClover3-YAP (green) was bleached using a 488-nm laser beam.

FIG. 2E illustrates domain structure and the intrinsically disordered tendency of SRC-1. PONDR (Predictor of Natural Disordered Regions) VSL2 assigned scores of disordered tendencies between 0 and 1 to the sequences (a score of more than 0.5 indicates disordered).

FIG. 2F illustrates fusing event of SRC-1 droplets. Scale bar represents 10 m. This assay was repeated three times with similar results.

FIG. 2G illustrates representative images of FRAP experiments of mScarlet-SRC1(red) in SF268 cells. The arrow indicates the region of photobleaching. After bleaching, the red puncta disappeared. With the passage of time, puncta gradually reorganized and returned to its original state. Scale bar, 5 μm.

FIG. 2H illustrates fusing event of mScarlet-SRC1(red) in SF268 cells. The arrow indicates the region of fusing. With time elapse, two droplets get closer, touch with each other and coalesce into a larger one. Scale bar, 5 μm.

FIG. 2I illustrates H1299 cells co-expressing YAP5SA, TEAD4-mTagBFP(blue) and mScarlet-SRC1(red) were stained with anti-RNA pol II-S5P (green, top) and anti-H3K27ac (green, bottom). Transcription activation markerH3K27ac and active RNA polymerase II phosphorylated at Ser 5 of CTD were enriched in SRC-1 co-occupied YAP/TEAD condensates. Scale bar, 5 μm.

FIG. 3A illustrates endogenous YAP was co-immunoprecipitated by SRC-1 antibody in SF268 cells.

FIG. 3B illustrates endogenous SRC-1 was co-immunoprecipitated by TEAD4 antibody in SF268 cells.

FIG. 3C illustrates schematic representation of Flag-SRC-1 mutants (N/M/C truncations).

FIG. 3D illustrates 293FT cells were transfected with YAP, TEAD4 and Flag-SRC-1 mutants (N/M/C truncations). Flag immunoprecipitates from cell lysates and total cell lysates were analyzed by western blotting.

FIG. 3E illustrates genomic views of YAP (SF268 cells) and SRC1 (K562 and LY2 cells) ChIP enrichment at the promoters of TEAD1, TEAD4, LATS2, and FZD1. Scale bar represents 2 kb.

FIG. 3F illustrates genomic views of YAP, TEAD and SRC-1 occupancy at gene promoters of AXL, CYR61, FZD1 and ATAD2 in SF268 and K562 cells.

FIG. 3G illustrates heatmap representing TEAD2/SRC-1-co-occupied peaks located within promoter or enhancer regions genome-wide.

FIG. 3H illustrates the Venn diagram depicting the overlap of SRC-1 and TEAD2 target genes by ChIP-seq in K562 cells.

FIG. 3I illustrates the occupancy of YAP and SRC-1 on YAP target regions (#1, #2 and #3) or control region. Data are representative of 2 independent experiments. Error bars indicate the s.e.m.

FIG. 4A illustrates live-cell images showing the localization of TEAD4-mTagBFP(blue) and mScarlet-SRC1 (red, top), ER-mEGFP(green) and mScarlet-SRC1 (red, bottom) in SF268 cells. The Pearson's correlation coefficient (PCC) was analyzed and the fluorescence intensity of two proteins along the white line in merged image was shown on the right. ER and YAP were both found to be co-localized with SRC-1 puncta.

FIG. 4B illustrates live-cell images showing the distribution of TEAD4-mTagBFP(blue), mScarlet-SRC1(red) and ER-mEGFP(green) in SF268 (Top) and MCF7(Bottom) cells. The comparison result of PCC between SRC1-TEAD4 and SRC1-ER in SF268 and MCF7 cells were shown on the right. SRC-1 was largely co-occupied with YAP condensates in YAP-driven SF268 cells, while distributed to ER condensates in ER-positive MCF7 cells.

FIG. 4C illustrates live-cell images of H1299 cells co-transfected with TEAD4-mTagBFP(blue), mScarlet-SRC1(red) and ER-mEGFP(green), together with or without YAP (5SA) and E2 treatment as indicated. Cartoon images depicting the localization of TEAD/YAP, ER and SRC-1 in E2 and YAP (5SA) condition were shown on the right. The quantification of fluorescence intensity of mScarlet-SRC1, ER-mEGFP and TEAD4-mTagBFP along the white line in the merged image was shown. SRC-1 could interplay between YAP and ER transcription condensates under different cell context.

FIG. 5A illustrates representative SRC-1 protein levels in in lung, liver, gastric, colon, breast and esophagus samples determined by IHC.

FIG. 5B illustrates representative images showing SRC-1 protein distribution in breast and lung samples determined by IHC.

FIG. 5C illustrates Kaplan-Meier plots of patients stratified by SRC-1 expression.

FIG. 5D illustrates cell proliferation of lung cancer cells A549/H1299/H661 transfected with siRNAs targeting SRC-1 and control siRNA. siCtrl n=3/siSRC1 n=9.

FIG. 5E. H1299 cells stably expressing dox-inducible SRC-1 shRNAs were inoculated into nude mice. Mice were treated with vehicle or dox-supplemented drinking water and the tumor volume is plotted as the mean±s.e.m. (n=10). *p<0.05, ***p<0.001.

FIG. 5F illustrates migration capacity of dox-inducible H1299-tet-on-shRNA-SRC1 cells treated w/o dox. The knock-down efficiency of two shRNAs was examined by immunoblots and radar map was used to record the trajectory of each cell. Statistical results of cell speed and directionality(D/d) of cells treated w/o dox was shown.

FIG. 5G illustrates microfluid experiments performed using H1299 cells stably expressing dox-inducible SRC-1 shRNA. Cells seeded on one side could penetrate the Matrigel and migrate to the other side under the FBS concentration gradient.

FIG. 5H illustrates transwell experiments using H1299 cells stably expressing dox-inducible SRC-1 shRNA. Crystal violet staining results of the bottom of transwell chambers were shown.

FIG. 5I illustrates colony formation assays in H1299 cells stably expressing dox-inducible SRC-1 shRNA treated w/o dox.

FIG. 6A illustrates representative SRC-1 and YAP protein levels in 120 NSCLC samples by IHC and the correlation of two protein levels was analyzed.

FIG. 6B illustrates representative SRC-1 and YAP protein levels in 120 NSCLC samples by IHC.

FIG. 6C illustrates representative images showing that SRC-1 and YAP exhibited similar distribution pattern.

FIG. 6D illustrates colony formation assay in BEAS-2B cells transfected with vehicle, YAP and/or SRC-1 expression plasmids. Quantification result of colony number in image is shown. Error bars show mean±s.e.m. (n=4), *p<0.05.

FIG. 6E illustrates zoomed images of BEAS-2B colonies transfected with YAP and/or SRC-1 expression plasmids. Scale bar represents 100 μm.

FIG. 6F illustrates time course images showing colonies of BEAS-2B cells overexpressing with YAP or YAP and SRC-1. Scale bar represents 100 μm.

FIG. 6G illustrates microscopic image of BEAS-2B cells overexpressing with YAP and SRC-1 on day 30. The red arrow indicates the bridged connections between colonies. Scale bar represents 100 μm.

FIG. 7A illustrates live-cell images showing the distribution of TEAD4-mTagBFP(blue) and mNeoGreen-SRC1(green) in nucleus of H1299 cells co-transfected with YAP5SA plasmids treated w/o 20 μM EVG. Quantification of fluorescence intensity of TEAD4-mTagBFP and mNeoGreen-SRC1 along the line indicated in the merged image was shown on the right. TEAD condensates which is reported to depend on YAP were used to characterize YAP/TEAD transcriptional condensates. SRC-1 co-occupied with YAP/TEAD transcription condensates. After EVG treatment, SRC1 phase separation was disrupted while the YAP/TEAD condensates remained intact. Scale bar, 5 μm.

FIG. 7B illustrates high-throughput screening results. 449 FDA-approved compounds were screened and evaluated by the z-factor of cell viability and luciferase activity (Blue dots: screened compounds; red dots: positive compounds). The screening criteria is luciferase z-factor <−4 and cell viability >−4. (Positive compound, PC: Fedratinib)

FIG. 7C illustrates qPCR analysis examining the expression of YAP-targeted CTGF and CYR61 was used to validate candidate positive compounds from the screening. Error bars show mean±s.e.m. (n=2).

FIG. 7D illustrates the qPCR analysis of YAP target genes in SF268 cells treated with DMSO or 20 M EVG for 24 h. Error bars show mean±s.e.m. (n=3).

FIG. 7E illustrates SF268 cells treated with EVG were subjected to RNA-seq analysis. GSEA enrichment plots of genes involved in Hippo/YAP signaling.

FIG. 7F illustrates SF268 cells were treated with 20 μM elvitegravir (EVG) or M fedratinib (PC) for the indicated times and subjected to western blotting analysis.

FIG. 7G illustrates SF268 cells treated with EVG or PC (fedratinib—an inhibitor of the non-receptor tyrosine kinase JAK2 which affects YAP nuclear translocation and phosphorylation) were examined by YAP phostag immunoblotting.

FIG. 7H illustrates A549 cells treated with DMSO, EVG or PC(Positive compound) were stained with anti-YAP antibody and DAPI. YAP translocated from nucleus to cytoplasm upon PC treatment, while remained in nucleus with EVG treatment. Scale bar represents 40 μm.

FIG. 7I illustrates the qPCR analysis of CTGF and CYR61 in LATS1/2 double knock-down cells treated with DMSO or EVG. Immunoblotting was used to assess the knock-down efficiency of LATS1/2.

FIG. 7J illustrates the association of YAP on YAP targets regions (#1:ANKRD1enhancer, #2:PAWRpromoter, #3:NPPBpromoter, #4:CTGF promoter) were analyzed by ChIP-qPCR from cells treated with DMSO or elvitegravir. Data are representative of 3 independent experiments. Error bars indicate the s.e.m. of triplicate qPCR data.

FIG. 7K illustrates the association of YAP on YAP target regions were analyzed by ChIP-qPCR in SF268 cells treated with EVG or DMSO. Error bars show mean±s.e.m. (n=3).

FIG. 7L illustrates analysis of indicated histone modifications at YAP targets regions #1 from SF268 cells treated with DMSO or EVG by ChIP-qPCR. Error bars show mean±s.e.m. (n=3), *p<0.05, ***p<0.001

FIG. 7 M illustrates immunofluorescence staining with anti-H3K27ac (red,top) and anti-RNA pol II-S5P (red, bottom) in H1299 cells co-expressing YAP5SA, TEAD4-mTagBFP(blue) and mNeoGreen-SRC1(green) treated with 20 μM EVG. EVG impaired the SRC-1 LLPS. Transcription active markerH3K27ac and active RNA polymerase II phosphorylated at Ser 5 of CTD were not enriched in SRC-1 condensates. Scale bar, 5 μm.

FIG. 7N illustrates purified Flag-SRC-1 protein was mixed with YAP and/or TEAD4 in the presence of increasing concentrations of EVG and was subjected to three independent pull-downs using anti-flag beads.

FIG. 7O illustrates time course living-cell imaging of mNeoGreen-SRC1(green) condensates upon EVG treatment. EVG impaired the LLPS of SRC-1 in time-dependent manner. Scale bar, 5 μm.

FIG. 7P illustrates living-cell imaging of mScarlet-SRC1 puncta treated with EVG. EVG impaired the LLPS of SRC-1 in time-dependent manner. Scale bar, 10 μm.

FIG. 7Q illustrates quantification result of high content image data for mScarlet-SRC-1 puncta in H1299 cells treated w/o 20 μM EVG. ***p<0.001.

FIG. 7R illustrates bio-layer interferometry (BLI) assays were performed with purified SRC-1 protein and EVG. Biotin labeled EVG was immobilized on the streptavidin biosensors and dipped into wells containing increasing concentrations of SRC-1 protein.

FIG. 7S illustrates structure of Biotin-EVG.

FIG. 7T illustrates the lysates from SF268 cells overexpressing Flag-SRC-1 were incubated with 20 μM biotin or biotin-EVG for streptavidin-beads pull-down assays.

FIG. 7U illustrates purified Flag-SRC1 protein was incubated with EVG-Biotin in the presence of increasing concentrations of EVG and further subjected to streptavidin-beads pull-down assays.

FIG. 7V illustrates cell proliferation (lung cancer cells and normal cells) affected by the treatment with elvitegravir. Error bars show mean±s.e.m. (n=3).

FIG. 7W illustrates the anti-proliferative effects of EVG in SRC1 knockdown and control cells. Data represent mean±s.e.m. (n=3).

FIG. 7X illustrates the colonies of BEAS-2B cells overexpressing with both YAP and SRC-1 treated w/o EVG.

FIG. 7Y illustrates schematic representation of the SRC-1 co-occupied YAP/TEAD transcriptional condensates. (Left) SRC-1 associates with YAP and TEAD to facilitate YAP target gene expression. The colocalization with H3K27ac and RNA pol II-S5P indicates SRC-1 co-occupied YAP/TEAD puncta are actively transcribed. (Right) EVG could antagonize YAP activity through disrupting the SRC1 phase-separation in SRC-1/YAP/TEAD transcription condensates, but had no effect on the LLPS of YAP/TEAD. The H3K27ac was not enriched in TEAD condensates upon EVG treatment, while pol II-S5P remained unchanged.

### DETAILED DESCRIPTION OF THE INVENTION

The following description of the disclosure is merely intended to illustrate various embodiments of the disclosure. As such, the specific modifications discussed are not to be construed as limitations on the scope of the disclosure. It will be apparent to one skilled in the art that various equivalents, changes, and modifications may be made without departing from the scope of the disclosure, and it is understood that such equivalent embodiments are to be included herein. All references cited herein, including publications, patents and patent applications are incorporated herein by reference in their entirety.

### Definitions

As used herein, the term “a,” “an,” “the” and similar terms used in the context of the present invention (especially in the context of the claims) are to be construed to cover both the singular and plural unless otherwise indicated herein or clearly contradicted by the context.

The term “agent” as used herein means any compound or substance such as, but not limited to, a small molecule, nucleic acid, polypeptide, peptide, drug, ion, etc. An “agent” can be any chemical, entity or moiety, including without limitation synthetic and naturally-occurring proteinaceous and non-proteinaceous entities. In some embodiments, an agent is nucleic acid, nucleic acid analogues, proteins, antibodies, peptides, aptamers, oligomer of nucleic acids, amino acids, or carbohydrates including without limitation proteins, oligonucleotides, ribozymes, DNAzymes, glycoproteins, siRNAs, lipoproteins, aptamers, and modifications and combinations thereof etc. In some embodiments, the agent is selected from the group consisting of a nucleic acid, a small molecule, a polypeptide, and a peptide. In certain embodiments, agents are small molecule having a chemical moiety. In some embodiments, the agent is sufficiently small to diffuse into a condensate. In some embodiments, the agent is less than about 4.4 kDa.

The term “small molecule” as used herein refers to a chemical molecule such as a compound that is not a peptide or a nucleic acid. In certain embodiments, a small molecule can be less than about 2 kilodaltons (kDa) in mass. In some embodiments, the small molecule is less than about 1.5 kDa, or less than about 1 kDa. In some embodiments, the small molecule is less than about 800 daltons (Da), 600 Da, 500 Da, 400 Da, 300 Da, 200 Da, or 100 Da. Often, a small molecule has a mass of at least 50 Da. In some embodiments, a small molecule is non-polymeric.

The terms “increased,” “increase”, “up-regulate” or “enhance” may be, for example, increase or enhancement by a statically significant amount. In some instances, for example, an element can be increased or enhanced by at least about 10% as compared to a reference level (e.g., a control), at least about 20%, at least about 30%, at least about 40%, at least about 50%, at least about 60%, at least about 70%, at least about 80%, at least about 90%, or at least about 100%, and these ranges will be understood to include any integer amount therein (e.g., 2%, 14%, 28%, etc.) which are not exhaustively listed for brevity. In other instances an element can be increased or enhanced by at least about 2-fold, at least about 3-fold, at least about 4-fold, at least about 5-fold at least about 10-fold or more as compared to a reference level.

The terms “decrease,” “reduce,” “suppress”, “down-regulate” and “inhibit” may be, for example, a decrease or reduction by a statistically significant amount relative to a reference (e.g., a control). In some instances an element can be, for example, decreased or reduced by at least 10% as compared to a reference level, by at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, up to and including, for example, the complete absence of the element as compared to a reference level. These ranges will be understood to include any integer amount therein (e.g., 6%, 18%, 26%, etc.) which are not exhaustively listed for brevity.

The terms “polynucleotide” or “nucleic acid” or “oligonucleotide” are used interchangeably, and refer to a chain of covalently linked nucleotides. The nucleotides may be deoxyribonucleotides or ribonucleotides, and modified or unmodified independent from one another. A polynucleotide may be single-stranded or double-stranded.

The terms “polypeptide” and “protein” are used interchangeably, and refer to a chain of amino acid residues covalently linked by peptide bonds. Proteins or polypeptide may include moieties other than amino acids (e.g., may be glycoproteins) and/or may be otherwise processed or modified. Those of ordinary skill will further appreciate that a protein can sometimes include more than one polypeptide chain, for example linked by one or more disulfide bonds or associated by other means.

The term “fragment” as used herein refers to partial sequence of the reference polypeptide or polynucleotide of any length. A fragment can still retain at least partial biological activities of the reference polypeptide.

The terms “variant” refers to a polypeptide having one or more amino acid residue changes or modification relative to a naturally occurring polypeptide.

“Functional equivalent” as used herein refers to a fragment, variant, or a fusion polypeptide of a naturally-occurring polypeptide (e.g., SRC-1 or YAP) that despite of having differences in their chemical structures retains at least partially biological functions of naturally-occurring polypeptide. In some embodiments, a functional equivalent retains at least 80%, 85%, 90%, 91%, 92%, 93%, 94%, 95%, 96%, 97%, 98%, 99% or 100% biological activity of naturally-occurring polypeptide.

The term “homologue” and “homologous” as used herein are interchangeable and refer to nucleic acid sequences (or its complementary strand) or amino acid sequences that have sequence identity of at least 80% (e.g., at least 85%, 88%, 90%, 91%, 92%, 93%, 94%, 95%, 96%, 97%, 98%, or 99%) to another sequences when optimally aligned.

“Percent (%) sequence identity” with respect to amino acid sequence (or nucleic acid sequence) is defined as the percentage of amino acid (or nucleic acid) residues in a candidate sequence that are identical to the amino acid (or nucleic acid) residues in a reference sequence, after aligning the sequences and, if necessary, introducing gaps, to achieve the maximum number of identical amino acids (or nucleic acids). Conservative substitution of the amino acid residues may or may not be considered as identical residues. Alignment for purposes of determining percent amino acid (or nucleic acid) sequence identity can be achieved, for example, using publicly available tools such as BLASTN, BLASTp (available on the website of U.S. National Center for Biotechnology Information (NCBI), see also, Altschul S. F. et al, J. Mol. Biol., 215:403-410 (1990); Stephen F. et al, Nucleic Acids Res., 25:3389-3402 (1997)), ClustalW2 (available on the website of European Bioinformatics Institute, see also, Higgins D. G. et al, Methods in Enzymology, 266:383-402 (1996); Larkin M. A. et al, Bioinformatics (Oxford, England), 23(21): 2947-8 (2007)), and ALIGN or Megalign (DNASTAR) software. Those skilled in the art may use the default parameters provided by the tool, or may customize the parameters as appropriate for the alignment, such as for example, by selecting a suitable algorithm.

An “isolated” substance has been altered by the hand of man from the natural state. If an “isolated” composition or substance occurs in nature, it has been changed or removed from its original environment, or both. For example, a polynucleotide or a polypeptide naturally present in a living animal is not “isolated,” but the same polynucleotide or polypeptide is “isolated” if it has been sufficiently separated from the coexisting materials of its natural state so as to exist in a substantially pure state.

The phrase “host cell” as used herein refers to a cell into which an exogenous polynucleotide and/or an expression vector has been introduced.

“Treating” or “treatment” of a condition as used herein includes preventing or alleviating a condition, slowing the onset or rate of development of a condition, reducing the risk of developing a condition, preventing or delaying the development of symptoms associated with a condition, reducing or ending symptoms associated with a condition, generating a complete or partial regression of a condition, curing a condition, or some combination thereof.

The term “tumor” or “cancer” are used interchangeably and refers to any diseases involving an abnormal cell growth and include all stages and all forms of the disease that affects any tissue, organ or cell in the body. The term includes all known cancers and neoplastic conditions, whether characterized as malignant, benign, soft tissue, solid, or hematologic, or of all stages and grades, including pre- and post-metastatic tumors. In general, cancers can be categorized according to the tissue or organ from which the tumor is located or originated and morphology of cancerous tissues and cells.

The term “oncogene” encompasses nucleic acids that, when expressed, can increase the likelihood of or contribute to cancer initiation or progression. Normal cellular sequences (“proto-oncogenes”) can be activated to become oncogenes by mutation and/or aberrant expression. In various embodiments an oncogene can comprise a complete coding sequence for a gene product or a portion that maintains at least in part the oncogenic potential of the complete sequence or a sequence that encodes a fusion protein. Oncogenic mutations can result, e.g., in altered (e.g., increased) protein activity, loss of proper regulation, or an alteration (e.g., an increase) in RNA or protein level.

The term “tag” as used herein includes, but is not limited to, detectable labels, such as fluorophores, radioisotopes, colorimetric substrates, or enzymes; heterologous epitopes for which specific antibodies are commercially available, e.g., FLAG-tag; heterologous amino acid sequences that are ligands for commercially available binding proteins, e.g., Strep-tag, biotin; fluorescence quenchers typically used in conjunction with a fluorescent tag on the other polypeptide; and complementary bioluminescent or fluorescent polypeptide fragments. A tag that is a detectable label or a complementary bioluminescent or fluorescent polypeptide fragment may be measured directly (e.g., by measuring fluorescence or radioactivity of, or incubating with an appropriate substrate or enzyme to produce a spectrophoto metrically detectable color change for the associated polypeptides as compared to the unassociated polypeptides). A tag that is a heterologous epitope or ligand is typically detected with a second component that binds thereto, e.g., an antibody or binding protein, wherein the second component is associated with a detectable label. In some embodiments, the detectable tag is a fluorescent tag. In some embodiments, both a condensate component and the agent comprise a detectable tag. In some embodiments, the component comprises a different detectable tag than the agent.

Condensate and Gene Expression Modulation

In addition to canonical membrane-bound organelles, eukaryotic cells contain numerous membraneless compartments, or condensates, that concentrate specific collections of proteins and nucleic acids. “Condensate”, as used herein, refers to non-membrane-encapsulated compartment formed by phase separation (including all stages of phase separation) of one or more of proteins and/or other macromolecules (such as RNA and/or DNA) based on their intrinsic physical properties. Condensates behave as phase-separated liquids, resulting in specific proteins and/or macromolecules being concentrated inside the condensates while other specific proteins and/or macromolecules are excluded. Condensates are liquid and reversible. Upon changes in cell physiology, such as a signaling event or a change in concentration of one of the macromolecules or other changes in the local environment, the condensates within the cell will change, altering their physical properties (e.g., formation, stability, composition, morphology, etc.), thereby modulating the biological activities associated with the condensates.

Emerging evidence has shown that gene expression is accompanied by the recruitment of large clusters of transcription complexes that form condensates through phase separation. Transcriptional condensates concentrate transcription factors, coactivators, the transcription and elongation machinery for spatial and temporal transcription control (Hnisz, D. et al., Cell 169, 13-23 (2017), Alberti, S., et al., Cell 176, 419-434 (2019). Lee, T. I. & Young, R. A. Cell 152, 1237-1251 (2013)). “Transcriptional condensates” as used herein refers to phase-separated multi-molecular assemblies that occur at the sites of transcription and are high density cooperative assemblies of multiple components that can include transcription factors, coactivators, chromatin regulators, DNA, non-coding RNA, nascent RNA, and RNA polymerase, histones and the like. Transcriptional condensates can also comprises an enzyme that alters, reads, or detects the structure of a chromatin component (e.g., a DNA methylase or demythylase, a histone methylase or demethylase, or a histone acetylase or de-acetylase that write, read or erase histone marks, e.g., H3K4mel or H3K27Ac). As many diseases are caused by, or associated with, alteration in their gene expression profiles, modulating transcriptional condensate, thereby altering transcriptional output of condensates, may afford a novel therapeutic intervention.

The present disclosure found that a transcriptional coactivator, SRC-1, participate in the expression of YAP target genes. SRC-1 and YAP together facilitate cancer progression. Moreover, the present disclosure found SRC-1 is capable of undergoing phase separation to form condensates. A SRC-1 condensate can be a transcriptional condensate that comprises additional transcriptional factors, coactivators (e.g., YAP) and the like. A transcriptional SRC-1 condensate occupies genes that are associated with oncogenic signaling pathways (e.g., YAP target genes). Modulation of SRC-1 condensate lead to changes in the expression pattern of these genes.

SRC-1 Condensate

In one aspect, the present disclosure provides a method of modulating transcription of one or more genes in a cell or in a subject, comprising modulating a transcriptional SRC-1 condensate comprising at least SRC-1, wherein the transcriptional SRC-1 condensate regulates transcription of the one or more genes.

“SRC-1” or “steroid receptor coactivator-1” as used herein refers to is a transcriptional coactivator that contains several nuclear receptor interacting domains and an intrinsic histone acetyltransferase activity. SRC-1 is also known as nuclear receptor coactivator 1 or NCOA1. SRC-1 assists nuclear receptors in upregulation of DNA expression. Upon recruiting to DNA promotion sites by ligand-activated nuclear receptors, SRC-1 acylates histones, making downstream DNA more accessible to transcription. As used herein, SRC-1 not only refers a naturally-occurring SRC-1, but may also refers to any functional equivalent thereof.

SRC-1 contains three distinct domains, including a b-HLH-PAS domain in the N terminus responsible for interactions with transcription factors and coactivators to activate gene transcription, a nuclear receptor (NR) interacting domain composed of 3 LXXLL motifs in the middle responsible for binding to and activating nuclear receptors, and two activation domains, AD1 and AD2 in the C terminus responsible for recruiting additional coactivators for histone modification and chromatin remodeling to enhance gene transcription. In addition to these structured domains, SRC-1 contains extensive intrinsic disordered domains (IDD) across the entire protein. “Intrinsic disordered domain” or “IDD” as used herein refers to regions in a protein that lacks a fixed or ordered secondary and tertiary structure. IDDs can range from fully unstructured to partially structured. In some embodiments, an IDD may be identified by the methods disclosed in Ali, M., & Ivarsson, Y. (2018). High-throughput discovery of functional disordered regions. Molecular Systems Biology, 14(5), e8377.

In some embodiments, the IDD has separate discrete regions. In some embodiments, the IDD is at least about 5, 10, 15, 20, 30, 40, 50, 60, 75, 100, 150, or more disordered amino acids (e.g., contiguous disordered amino acids). In some embodiments, an amino acid is considered a disordered amino acid if at least 75% of the algorithms employed by D2P2 (Oates et al., 2013, Nucleic Acids Res. 41, D508-16.) predict the residue to be disordered.

In some embodiments, SRC-1 undergoes phase separation to form condensates in which SRC-1 is highly concentrated both in vitro and in cells. SRC-1 condensate as used herein refers to a condensate that comprises at least SRC-1. A SRC-1 condensate can be a homotypic condensate that comprises only SRC-1, or a heterotypic condensate that comprise additional components. In some embodiments, SRC-1 condensates occupy the sites of active gene transcription in cells. In some embodiments, SRC-1 condensate is a transcriptional SRC-1 condensate.

In some embodiments, SRC-1 condensate comprise at least one additional component. “Component” with regard to a condensate refers to a molecule that can be found associated with or incorporated into a condensate under physiological or pathological conditions. In some embodiments, a component of SRC-1 condensate is a macromolecule that can undergo phase separation by itself. In some embodiments, a component of SRC-1 condensate is a macromolecule that by itself, cannot undergo phase separation, but reach high local concentration by interacting with SRC-1. In some embodiments, a component within the transcriptional SRC-1 condensate is a macromolecule that typically interacts with or binds to SRC-1, such as a nuclear receptor, a transcription factor, a transcription coactivator, a histone or a RNA polymerase. In some embodiments, the transcriptional SRC-1 condensate comprises multiple components in addition to SRC-1. “Transcription factor” or TF, as used herein, is a protein that regulates transcription by binding to a specific DNA sequence. TFs generally contain a DNA binding domain and activation domain. In some embodiments, the TF is regulated by a signaling factor (e.g., transcription is modulated by TF interaction with a signaling factor). “Transcriptional coactivator”, as used herein, refers to a protein or complex of proteins that interacts with transcription factors to stimulate transcription of a gene. In some embodiments, the TF is a nuclear receptor. “Nuclear receptor” or NR as used herein, refer to a members of a large superfamily of evolutionarily related DNA-binding transcription factors that exhibit a characteristic modular structure consisting of five to six domains of homology (designated A to F, from the N-terminal to the C-terminal end). The activity of NRs is regulated at least in part by the binding of a variety of small molecule ligands to a pocket in the ligand-binding domain.

In some embodiments, the transcriptional SRC-1 condensate further comprises a first component capable of interacting with SRC-1. In some embodiments, the first component comprises Yes-associated protein (YAP), Estrogen receptor (ER), androgen receptor (AR), vitamin D receptor(VDR) and AP-1.

“Yes-associated protein” or “YAP” (also known as YAP1 or YAP65) as used herein, is a transcriptional coactivator that plays essential role in promoting cell proliferation, development and stem-cell fate. YAP activates the transcription of genes involved in cell proliferation and suppressing apoptotic genes. YAP is inhibited in the Hippo signaling pathway which allows the cellular control of organ size and tumor suppression. In mammals, a kinase cascade including MST1/2 and LATS1/2 phosphorylate YAP to prevent its nuclear translocation and subsequent association with the TEA-domain transcription factors TEAD1-4 (collectively, TEAD) in the canonical Hippo pathway. YAP together with TEAD (or other transcriptional factors) induce the expression of a variety of genes, including connective tissue growth factor (CTGF), Gli2, Birc5, Birc2, fibroblast growth factor 1(FGF1), ankyrin repeat domain-containing protein (ANKRD), cysteine rich angiogenic inducer 61(CYR61), TGB2, AREG, Foxf2, IGFBP3, RASSF2 and amphiregulin. Many of these genes activated by YAP mediate cell survival and proliferation. Therefore YAP acts as an oncogene. Aberrant YAP activation is prevalent in diverse types of human solid cancers.

“Estrogen receptor” or ER as used herein refers to a group of nuclear receptors, including the nuclear estrogen receptors ER-alpha and ER-beta, that are activated by the hormone estrogenic hormones. “Androgen receptor” or AR as used herein refers to a member of nuclear receptors that is activated by the androgenic hormones. Once activated by estrogenic or androgenic hormones, the ER or AR is able to translocate in the nucleus and bind to DNA to regulate the activation of different genes. “Vitamin D receptor” or VDR as used herein refers to a member of nuclear receptors that is activated by the active form vitamin D and forms a heterodimer with the retinoid-X receptor upon activation. “Activator protein 1” or AP-1 as used herein refers to a transcription factor that regulates gene expression in response to a variety of stimuli, including cytokines, growth factors, stress, and bacterial and viral infections. AP-1 is usually a heterodimer composed of proteins belonging to the c-Fos, c-Jun, ATF and JDP families.

In some embodiments, the transcriptional SRC-1 condensate further comprises a second component interacting with the first component. In some embodiments, the first component comprises YAP. In some embodiments, the second component interacts with YAP. In some embodiments, the second component comprises a TEA-domain transcription factor. In some embodiments, the TEA-domain transcription factor comprises TEAD1, TEAD2, TEAD3, TEAD4 or any combination thereof.

“TEA-domain transcription factor” or TEAD as used herein refers to a group of transcription factors, which is comprised of TEAD1, TEAD2, TEAD3 and TEAD4, that act as the final nuclear effectors of the Hippo pathway that regulate cell growth, proliferation, and tissues homeostasis via their transcriptional target gene. TEAD activities have been serving as the functional readout of the Hippo-YAP pathway. Each family member of TEAD has multiple names TEAD1 (TEF-1/NTEF), TEAD2 (TEF-4/ETF), TEAD3 (TEF-5/ETFR-1), and TEAD4 (TEF-3/ETFR-2/FR-19). All TEAD share a highly conserved DNA-binding domain in the N-termini and a transactivation domain for interacting with YAP in the C-termini.

In some embodiments, the transcriptional SRC-1 condensate further comprises a RNA polymerase. In some embodiments, the RNA polymerase is RNA polymerase II.

“RNA polymerase II” or Pol II as used herein refers to a multiprotein complex of 12 subunits that transcribes DNA into pre-mRNA and most small nuclear RNA and microRNA. A wide range of transcription factors are required for Pol II to bind to upstream gene promoters and begin transcription. The synthesis of pre-mRNA by RNA polymerase II (Pol II) involves the formation of a transcription initiation complex and a transition to an elongation complex. The large subunit of Pol II contains an intrinsically disordered C-terminal domain (CTD), which is phosphorylated by cyclin-dependent kinases (CDKs) during the initiation-to-elongation transition, thus influencing the CTD's interaction with different components of the initiation.

In some embodiments, the transcriptional SRC-1 condensate further comprises a histone. Eukaryotic transcription is regulated by chromatin structure, whose alterations are mediated by conserved post-translational histone tail modifications. Histone tail modification includes, without limitation, acetylation, methylation, phosphorylation, and ubiquitination. Histone acetylation, typically catalyzed by histone acetyltransferase (e.g., SRC-1) that acetylates the lysine residues within the histone tail, decreases the interaction of histone with DNA, thereby transforming the condensed chromatin into a more relaxed structure to facilitate greater levels of gene transcription. In some embodiments, the histone comprises H3K27ac.

In some embodiments, the transcriptional SRC-1 condensate is associated with one or more genes. These genes typically refer to specific genes, the locus of which can be occupied by a SRC-1 condensate in a cell. The localization of a SRC-1 condensate to these genetic loci may require structured TF-DNA interaction (e.g., interaction mediated by TEAD that is incorporated in a SRC-1 condensate) and/or IDD mediated interactions. In some embodiments, the one or more genes associated with a transcriptional SRC-1 condensate is a gene targeted by YAP together with TEAD (i.e., YAP target genes, see description below).

In some embodiments, the one or more genes associated with a transcriptional SRC-1 condensate comprise the one or more genes associated with oncogenic signaling pathways. In some embodiments, the transcription of one or more genes are associated with a hallmark of a disease such as cancer. In some embodiments, the one or more genes associated with a transcriptional SRC-1 condensate comprise one or more oncogenes. Exemplary oncogenes include MYC, SRC, FOS, JUN, MYB, RAS, ABL, HOXI1, HOXI1 1L2, TAL1/SCL, LM01, LM02, EGFR, MYCN, MDM2, CDK4, GLI1, IGF2, activated EGFR, mutated genes, such as FLT3-ITD, mutated of TP53, PAX3, PAX7, BCR/ABL, HER2/NEU, FLT3R, FLT6-ITD, SRC, ABL, TAN1, PTC, B-RAF, PML-RAR-alpha, E2A-PRX1, and NPM-ALK, as well as fusion of members of the PAX and FKHR gene families. Other exemplary oncogenes are well known in the art. In some embodiments the oncogene is selected from the group consisting of c-MYC and IRF4. In some embodiments the gene encodes an oncogenic fusion protein, e.g., an MLL rearrangement, EWS-FLI, ETS fusion, BRD4-NFTT, NEGR98 fusion.

SRC-1 Condensate Modulation

The transcriptional SRC-1 condensate can regulate the transcription of one or more genes associated with the SRC-1 condensate. Regulating transcription of a gene can, for example, include one or more of the following events: increasing or decreasing the rate or frequency of gene transcription, increasing or reducing inhibition of gene transcription, and increasing or decreasing mRNA transcription initiation, mRNA elongation, or mRNA splicing activity.

In some embodiment, the transcription of one or more genes is modulated by modulating the transcriptional SRC-1 condensate. As used herein “modulating” (and verb forms thereof, such as “modulates”) means causing or facilitating a qualitative or quantitative change, alteration, or modification. Without limitation, such change may be an increase or decrease in a qualitative or quantitative aspect. In some embodiments, the transcriptional SRC-1 condensate is modulated by modulating or reducing formation, composition, stability, and/or activity of the transcriptional SRC-1 condensate. In some embodiments, modulating a condensate includes one, two, three, four or all five of modulating formation, composition, stability and/or activity of a condensate. In some embodiments, modulating a condensate also includes modulating the morphology or shape of the condensate, and/or modulating cell signaling cascade that involves one or more component associated with the condensate.

“Formation” with regard to condensate, as used herein, refers to the generation of a condensed biological assembly with well-delineated physical boundaries, but without lipid membrane barriers. The formation of condensate can be driven by phase separation, in particular phase separation of a driver protein (e.g., an intrinsically-disordered protein or comprises intrinsically-disordered regions that is capable of phase separating on its own in vitro).

In some embodiments, the formation of SRC-1 condensate is driven by phase separation of SRC-1. In some embodiments, disturbing the ability of SRC-1 to phase separate impairs the formation of SRC-1 condensate. In some embodiments, the formation of SRC-1 condensate is driven by phase separation of a component of the SRC-1 condensate but not SRC-1. In some embodiments, the formation of SRC-1 condensate is driven by YAP or TEAD. In some embodiments, modulating the formation of a SRC-1 condensate includes increasing or decreasing the rate of formation or whether or not formation occurs.

“Composition” as used herein, refers to the collection of components associated within a condensate. A transcriptional condensate typically comprises multiple components, including proteins and/or nucleic acids. It is not necessary for each of the components of a condensate to interact with a driver component. In some embodiments, the SRC-1 condensate comprises, in addition to SRC-1, a first component (e.g., YAP) that interacts with Src-1, and a second component (e.g., TEAD) that interact with the first component, wherein the second component may or may not interact with SRC-1.

In some embodiments, the composition of a condensate changes in response to changes to the environment or a stimuli applied to the condensate (e.g., pH, protein concentrations, addition of further micro- or macro-molecules, etc.). When individual components are absent from a transcriptional condensate, it may be rendered non-functional (i.e., incapable of productive transcription). Additionally, incorporating novel components into existing condensates may modify, attenuate, or amplify their output. In some embodiments, modulating the composition of a condensate includes increasing or decreasing the level of a component associated with the condensate.

In some embodiments, the transcriptional SRC-1 condensate is modulated by modulating the amount or level of SRC-1 or a component associated with the transcriptional condensate (e.g., a first component or a second component as described herein). In some embodiments, the amount or level of SRC-1 or the component associated with the transcriptional condensate is modulated by contact with an agent that reduces or eliminates the level of SRC-1. The agent is not limited and may be any agent described herein.

“Stability” as used herein refers to the property of a condensate that when disturbed from a condition of equilibrium to restore its original condition. The stability of a condensate can be reflected by the maintenance or dissolution (either partial or complete) of a condensate. Maintenance refers to the preserving of the composition and physical properties of a condensate. Dissolution refers to the disassembly of a condensate, either partially or completely. In some embodiments, modulating the stability of a condensate includes increasing or decreasing the rate of condensate maintenance or dissolution, or promoting or suppressing condensate dissolution.

“Activity” as used herein refers to the activity of SRC-1 condensate in regulating the transcription of the genes that are associated with the SRC-1 condensate. In some embodiments, the activity of a condensate is correlated with the composition or stability of a condensate. Changes in the composition or stability of a condensate may affect the activity of the condensate. In some embodiments, modulating condensate activity includes modifying the transcriptional activity of a condensate.

SRC-1 Condensate Inhibitor

In some embodiments, the transcriptional SRC-1 condensate is modulated by contacting with a SRC-1 condensate inhibitor. “SRC-1 condensate inhibitor” as used herein refers to an agent capable of down regulating the level or activity of a SRC-1 condensate. In certain embodiments, the SRC-1 condensate inhibitor disturbs, reduces, or suppresses the formation, composition, stability, or activity of the transcriptional SRC-1 condensate.

In some embodiments, the SRC-1 condensate inhibitor reduces formation, composition or stability of a SRC-1 condensate. For example, a SRC-1 condensate inhibitor may disrupt the interactions required for formation or maintenance or of the SRC-1 condensate, or may induce dissolution of the SRC-1 condensate, or may induce sequestration of SRC-1 outside of the SRC-1 condensate, or may cause changes in composition in the SRC-1 condensate that affect its stability or reduce SRC-1 in the SRC-1 condensate.

In some embodiments, the SRC-1 condensate inhibitor reduces or eliminates interactions between SRC-1 and one or more components in the transcriptional condensate, optionally in a SRC-1 selective manner. In some embodiments, the SRC-1 condensate inhibitor reduces or eliminates binding of SRC-1 to one or more components in the transcriptional condensate, optionally in a SRC-1 selective manner. In some embodiments, the one or more components comprises YAP, Estrogen receptor (ER), androgen receptor (AR), vitamin D receptor (VDR) and AP-1. “SRC-1 selective” manner as used herein means that the SRC-1 condensate inhibitor affects SRC-1 dependent interactions or activities, but does not significantly affect SRC-1 independent interactions or activities within the condensate. For example, the SRC-1 inhibitor may inhibit SRC-1 itself and/or an interaction of SRC-1 with another component of the condensate, but does not inhibit any interactions of certain component other than SRC-1 in the condensate, either as a single component interaction or interactions among these components other than SRC-1.

In some embodiment, the interaction between SRC-1 and one or more components in the transcriptional condensate is mediated by the IDD of SRC-1. In some embodiments, the binding between SRC-1 and one or more components in the transcriptional condensate is mediated by a structured domain (e.g., a b-HLH-PAS domain, AD1, AD2 or NR interaction domain) of SRC-1. Without bound to any theory, phase separation or condensate formation is driven by multivalent interaction either involving specific, high-affinity interactions mediated by structured domains, or weak interactions mediated by IDD. As used herein, “valency” refers to both the number of different binding partners for a component and the strength of the binding to one or more binding partners. SRC-1 comprises both structured domains and extensive IDDs that are capable of intermediating multivalent interactions with multiple partners. Thus, modulating either the interaction mediated by the IDDs of SRC-1 or the binding mediated by the structured domains of SRC-1 can modulating the transcriptional SRC-1 condensate. In some embodiments, the modulation decreases the valency of SRC-1 so as to suppress or prevent condensate formation.

In some embodiments, the SRC-1 condensate inhibitor interacts with an IDD of SRC-1. In some embodiments, the SRC-1 condensate inhibitor binds to a non-IDD region of SRC-1. In some embodiments, the inhibitor competes with a component associated with SRC-1 condensate for binding or interacting with SRC-1. For example, an inhibitor can displace a component from interacting or binding with SRC-1 and suppress the formation of SRC-1 condensate. In some embodiments, the SRC-1 condensate inhibitor allosterically induces a conformational change in the IDD. An inhibitor that acts allosterically can binds or interacts with a region in SRC-1 and causes conformational changes outside or distal to the interacting or binding region.

In some embodiments, the SRC-1 condensate inhibitor sequesters SRC-1 outside the transcriptional condensate. For example, the SRC-1 condensate inhibitor may bind to SRC-1 and prevent it from being incorporated into the condensate. For another example, a second SRC-1 condensate may be induced to form by adding a suitable agent (e.g., exogenously added small molecule, protein, DNA or RNA). The sequestration of SRC-1 outside the transcriptional condensate or in a second SRC-1 condensate modulates the transcriptional SRC-1 condensate by restricting access to the SRC-1.

In some embodiments, the SRC-1 condensate inhibitor sequesters SRC-1, without significantly dissolving the transcriptional condensate having reduced or no SRC-1. In some embodiments, despite of SRC-1 sequestration, the transcriptional condensate still exist and can functionally regulate transcriptional activities, except that it has reduced or no SRC-1 dependent transcriptional activity. For example, the transcriptional condensate may comprise at least YAP and TEAD (but no or reduced SRC-1), and retains transcriptional activity mediated by YAP and TEAD but independent of SRC-1. Such transcriptional activity of the condensate can be determined using any suitable methods, for example, by detecting active transcription activities at the DNA or RNA level (e.g. by DNA-FISH or RNA-FISH), by detecting expression of YAP-regulated gene expression using for example, reporter gene assay, quantitative PCR, western blot, or the like.

In some embodiments, the cell or the subject has an elevated expression level of SRC-1 relative to a reference level. SRC-1 has been associated with breast cancer previously. It is herein disclosed that elevated expression SRC-1 in non-small cell lung cancer is correlated with malignant features and poor prognosis. Therefore, perturbation of transcriptional SRC-1 condensate may lead to cancer cell death. In some embodiments, a transcriptional SRC-1 condensate inhibitor interacts or binds to SRC-1 to disturb, reduce or suppress the formation, composition, stability and/or activity of the transcriptional SRC-1 condensate.

In some embodiments, the SRC-1 condensate inhibitor comprises a peptide, nucleic acid, or small molecule. A peptide, nucleic acid or a small chemical molecule can interact with either a structured domain or IDD of SRC-1. The interaction would be expected to influence condensate formation, composition, stability or activity and thereby result in altering the transcription output of a transcriptional SRC-1 condensate. Thus, the expression of one or more genes can be influenced by modulating a transcriptional condensate with a SRC-1 condensate inhibitor that comprises a peptide, nucleic acid, or small molecule.

In some embodiments, the transcriptional SRC-1 condensate is a multivalent, comprising at least one anchor moiety and at least one disruptor moiety. The “disruptor” moiety weakly interact with components of the condensate to disrupt or alter the nature of the interaction. The “anchor” moiety has strong affinity for a more structured region of a protein that is in or near the condensate, thus serving to concentrate the disruptor molecule in or near the condensate (e.g., a transcriptional SRC-1 condensate). In some embodiments, a SRC-1 condensate inhibitor interacts with an IDD of SRC-1 and further binds to a structured domain of SRC-1 (e.g, AD1, AD2, NR interaction domain or b-HLH-PAS domain). In some embodiments, the SRC-1 condensate inhibitor binds to an IDD of SRC-1 and further binds to an additional component associated with the transcriptional condensate.

In some embodiments, the SRC-1 condensate inhibitor decreases level of transcriptional SRC-1 condensate by at least 30% (e.g., at least 10%, 20%, 30%, 40%, 50%, 60%, 70%, or 80%) at a concentration of no more than 20 uM (e.g., no more than 10 uM, 5 uM, 1 uM, 500 nM, 200 nM, 100 nM, 50 nM, 20 nM, or 10 nM).

The level of transcriptional SRC-1 condensate can be indicated by the amount of a transcriptional SRC-1 condensate. The amount of a transcriptional SRC-1 condensate can be measured by the methods disclosed herein or any suitable methods known in the art. For example, SRC-1 can be conjugated with a detectable label such as a fluorescent molecule, which allows SRC-1 condensate to be visualized under microscope as a punctum, and the fluorescence intensity correlates to the level of SRC-1 condensate. If SRC-1 is not associated with a condensate, the fluorescence intensity generally appears evenly distributed in nucleus or cytosol. The level of SRC-1 condensates can be further determined by a suitable method, for example, by counting the number of SRC-1 condensates visualized under a selected field under the microscope, or by calculating the increased florescence intensity signal relative to the background signal, or by calculating the area where florescence intensity is above a predetermined threshold.

Alternatively, the level of transcriptional SRC-1 condensate can be indicated by the activity of a transcriptional SRC-1 condensate. The activity of SRC-1 condensate can be determined by any suitable methods, including, without limitation, by determination of expression level of a gene associated with SRC-1 condensate (e.g., by methods such as qPCR, RNA-seq) and/or the acetylation level of histones associated with SRC-1 condensate (e.g., by methods such as ChIP-Qpcr or ChIP-seq).

In some embodiments, the SRC-1 condensate inhibitor comprises elvitegravir (EVG), or competes with EVG for binding to SRC-1, or induces a conformational change in SRC-1 at least comparable to that induced by EVG. EVG, developed by Gilead Sciences and marketed under If the brand name Vitekta, is an integrase inhibitor for the treatment of HIV infection. Its IUPAC name is 6-[(3-chloro-2-fluorophenyl)methyl]-1-[(2S)-1-hydroxy-3-methylbutan-2-yl]-7-methoxy-4-oxoquinoline-3-carboxylic acid and has the following structure

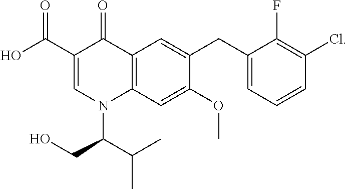 [SMILES: COc1cc2c(cc1Cc1cccc(Cl)c1F)c(=O)c(C(=O)O)cn2[C@H](CO)C(C)C]

The ability to “compete for binding” as used herein refers to the ability of an agent (e.g., a small molecule) inhibit the interaction between two molecules (e.g. EVG and SRC-1) to any detectable degree (e.g. by at least 85%, or at least 90%, or at least 95%). Those skilled in the art will recognize that it is possible to determine, without undue experimentation, if a given agent competes for binding or interacting to SRC-1 with EVG.

In some embodiments, the SRC-1 condensate inhibitor has comparable activity to EVG or higher activity than EVG in decreasing level of transcriptional SRC-1 condensate.

Modulation of YAP Target Genes

In another aspect, the present disclosure provides a method of modulating transcription of one or more YAP target genes in a cell or in a subject, comprising modulating SRC-1 by a SRC-1 inhibitor.

“YAP target genes” as used herein refers to genes, the expression (e.g., the activation or suppression of transcription) of which are under the regulation of YAP together with TEAD (or with other transcriptional factors). In some embodiment, the transcription of YAP target genes are activated or up-regulated by YAP. Many of YAP target genes mediate cell proliferation and survival, including genes driving G1/S phase transition, DNA replication and repair, nucleotide metabolism, and mitosis cell. This reflects the potent pro-tumorigenic activity of YAP in promoting cell growth and preventing cell senescence. In some embodiments, YAP target genes also include those encoding upstream regulators of the Hippo pathway and of the integrin and cytoskeletal machinery. These genes can limit or reinforce the activity of YAP. In some embodiments, the YAP target genes are associated with oncogenic pathways. In some embodiments, the YAP target genes are oncogenes.

In some embodiments, YAP target genes comprises connective tissue growth factor (CTGF), Gli2, Birc5, Birc2, fibroblast growth factor 1(FGF1), ankyrin repeat domain-containing protein (ANKRD), cysteine rich angiogenic inducer 61(CYR61), TGB2, AREG, Foxf2, IGFBP3, RASSF2 and amphiregulin. In some embodiments, the one or more YAP target genes are selected from the group consisting of ANKRD1, CTGF, and CYR61.

Given that YAP is associated with a transcriptional condensate comprising SRC-1, it is contemplated herein to modulate transcriptional SRC-1 condensate so as to modulate the transcription of one or more YAP targeted genes. In some embodiments, a SRC-1 condensate inhibitor or a SRC-1 inhibitor is used for modulating the transcription of one or more YAP targeted genes.

In some embodiments, the cell or the subject has an elevated expression level of SRC-1 relative to a reference level. It has been reported that SRC-1 is associated with breast cancer and prostate cancer (Redmond, A. M., et al., Clin Cancer Res 15, 2098-2106 (2009)). It is herein disclosed that elevated expression SRC-1 in non-small cell lung cancer is correlated with malignant features and poor prognosis, as well as an elevated expression of YAP. In some embodiment, the cell or the subject has a co-elevated expression level of SRC-1 and YAP relative to a reference level. A reference level can be obtained from one or more reference samples (e.g., samples obtained from healthy subjects, or from healthy tissues from a patient). A reference level can also be obtained from a database, which includes a collection of data, standard, or level from one or more reference samples. In some embodiments, such collection of data, standard or level are normalized.

### SRC-1 Inhibitor

“SRC-1 inhibitor” as used herein refers to an agent capable of down regulating the level or activity of a SRC-1. The SRC-1 inhibitor includes but not limited to SRC-1 condensate inhibitor. For example, a SRC-1 inhibitor reduce the expression level of SRC-1 but does not affect the phase separation behavior of SRC-1. In some embodiments, the SRC-1 inhibitor comprises a peptide, nucleic acid, or small molecule.

In some embodiments, the nucleic acid comprises an oligonucleotide specifically hybridizable to SRC-1 mRNA, or a polynucleotide encoding the oligonucleotide.

The oligonucleotides of the present disclosure are designed to hybridize under stringent conditions to SRC-1 mRNA. “Stringent condition” as used herein refers to a condition under which a sequence will hybridize to its target sequence (i.e. complementary sequence) but will not hybridize to other, non-complementary sequences. Stringent conditions are sequence-dependent and are different in different circumstances.

In some embodiments, the oligonucleotide is complementary to a target portion in SRC-1 mRNA and inhibit its expression or function. The oligonucleotide can hybridize to any suitable target portion of SRC-1 mRNA. The term “portion” as used herein refer to a defined number of contiguous nucleotides of an oligonucleotide or nucleic acid. A suitable target portion of SRC-1 mRNA can be determined by a skilled person in the art, for example, to have a sufficiently unique sequence so as to minimize undesirable off-target binding, and/or to be sufficiently accessible to oligonucleotide binding despite of the secondary or tertiary structure of SRC-1 mRNA. In some embodiment, the target portion of SRC-1 mRNA is of 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more nucleotides in length, or is between a range defined by any two of the above lengths. 100% complementarity between the sequence of the oligonucleotide and the targeted portion of SRC-1 mRNA may not be required. In certain embodiments, the oligonucleotide comprises a sequence at least, 70%, 75%, 80%, 85%, 86%, 87%, 88%, 89%, 90%, 91%, 92%, 93%, 94%, 95%, 96%, 97%, 98%, 99%, or 100% complementary to the targeted portion of SRC-1 mRNA.

In some embodiments, the oligonucleotide targeting SRC-1 mRNA is at least 8 to 80, 10 to 80, 12 to 50, 15 to 30, 18 to 24, 19 to 22, or 20 nucleotides in length.

In some embodiments, the oligonucleotides can be chemically modified. The modifications encompass substitutions or changes to internucleoside linkage, sugar moiety of a nucleotide or nucleobase of a nucleotide. Modified oligonucleotides can have desirable properties such as, for example, enhanced cellular uptake, enhanced affinity for nucleic acid target, increased stability in the presence of nucleases, or increased inhibitory activity. Chemically modified nucleotides can also be employed to increase the binding affinity of a shortened or truncated oligonucleotide for its target nucleic acid. It is not necessary for all positions in a given oligonucleotide to be uniformly modified, and in fact more than one of the aforementioned modifications may be incorporated in a single oligonucleotide or even at within a single nucleoside within an oligonucleotide.

The oligonucleotides may be covalently linked to one or more moieties or conjugates which enhance the activity, cellular distribution or cellular uptake of the resulting antisense oligonucleotides. The oligonucleotides can also be modified to have one or more stabilizing groups that are generally attached to one or both termini of the oligonucleotides to enhance properties such as, for example, nuclease stability.

In some embodiments, the oligonucleotide comprises siRNA, shRNA, miRNA, or antisense oligonucleotide.

Antisense oligonucleotides is a single-stranded oligonucleotides (e.g., a single-stranded DNA oligonucleotides) that binds to a target RNA in a sequence-specific manner to inhibit gene expression, modulate splicing of a precursor messenger RNA, or inactivate microRNAs. The optimal length of the antisense oligonucleotide may very (e.g., 12-18 nucleotides in length) while ensuring that its target sequence is unique in the transcriptome (Seth (2009) J Med Chem 52: 10-13). In some embodiments, the antisense oligonucleotides include one or more modifications as described herein.

Small interfering RNA (“siRNA”) or a small hairpin RNA (“shRNA”) both comprise double-stranded RNA (dsRNA) structure that cause repression or degradation of single-stranded target RNAs in a sequence specific manner (i.e. RNA interference).

siRNA can be assembled from two separate oligonucleotides, where one strand is the sense strand and the other is the antisense strand, where the antisense and sense strands are self-complementary and form a duplex or double stranded structure; the antisense strand comprises nucleotide sequence that is complementary to at least a portion of a target nucleic acid molecule. In some embodiments, the double-stranded structure is about 15 to about 30, e.g., about 15, 16, 17, 18, 19, 20, 21, 22, 23, 24, 25, 26, 27, 28, 29 or 30 base pairs. In some embodiments, the siRNA has a 3′ overhangs on each strand. In the format of shRNA, a single oligonucleotide, the self-complementary sense and antisense regions are linked by means of nucleic acid based or non-nucleic acid-based linker(s). In some embodiments, a shRNA having a sense region, an antisense region and a loop region. The loop region is generally between about 2 and about 10 nucleotides in length. Following post-transcriptional processing, the shRNA can be converted into a siRNA by a cleavage event mediated by the enzyme Dicer. In some embodiments, the siRNA and shRNA include one or more modifications as described herein.

In some embodiments, the SRC-1 inhibitor is a SRC-1 mimetic. The terms “mimetic,” refer to a peptide, partial peptide or non-peptide molecule that mimics the tertiary binding structure or activity of a selected native peptide (e.g., SRC-1) or protein functional domain (e.g., structured domain or IDD of SRC-1). These peptide mimetics include recombinantly or chemically modified peptides, as well as non-peptide agents such as small molecule drug mimetics. In some embodiments, a SRC-1 mimetic interacts with SRC-1, or a component (e.g., a first component as described herein) associated a transcriptional SRC-1 condensate to disturb, reduce or suppress the formation, composition, stability and/or activity of the condensate. In some embodiments, a SRC-1 mimetic is able to sequester SRC-1 from a transcriptional SRC-1 condensate, for example, into a second SRC-1 condensate.

Methods of Treating a Disease or Condition

In another aspect, the present disclosure provides a method of treating a YAP-associated disease or condition in a subject, comprising administering to the subject a pharmaceutically effective amount of a SRC-1 inhibitor.

In another aspect, the present disclosure provides a method of treating a SRC-1 condensate associated disease or condition, comprising administering to the subject a pharmaceutically effective amount of a SRC-1 condensate inhibitor.

YAP, as transcriptional coactivator, shuttles between the cytoplasm and the nucleus in response to the Hippo pathway. In the nucleus, YAP pair with the TEAD family to regulate the expression of genes that are involved in promoting cell proliferation, organ overgrowth, survival to stress and so forth. Enhanced levels and activity of YAP are observed in many cancers, where they sustain tumor growth, drug resistance and malignancy. In some embodiments, a YAP-associated disease or condition is a cancer.

SRC-1 was initially identified as a steroid receptor coactivator. Ever since, SRC-1 is found to bind across many families of transcription factors to orchestrate and regulate complex physiological reactions in the development and maintenance of normal tissues, as well as to cellular proliferation and tumor growth. It is herein disclosed that SRC-1 is able to undergo phase separation to form a SRC-1 condensate. In some embodiments, a SRC-1 condensate associated disease or condition is characterized by the activation of ER, AR, VDR or AP-1. In some embodiments, a SRC-1 condensate associated disease or condition comprises neurological disorders, cardiac development diseases, inflammatory diseases, metabolic disorders, circadian disorders, or cancer.

It is herein disclosed that SRC-1 facilitates YAP transcriptional activity, and a SRC-1 condensate in cell can incorporate YAP. In some embodiments, a SRC-1 condensate associated disease or condition is characterized by enhanced level and/or activity of YAP.

In some embodiments, the disease or condition is characterized in an elevated expression level of SRC-1 relative to a reference level. SRC-1 has been associated with breast cancer and prostate cancer previously. It is herein disclosed that elevated expression SRC-1 in non-small cell lung cancer is correlated with malignant features and poor prognosis.

In some embodiments, the disease or condition is associated with aberrant expression of an oncogene. “Aberrantly expression” is used to indicate that the gene expression is detectably different from a reference level that is typical of that found in normal cells (e.g., normal cells of the same cell type or, for cultured cells, cultured cells under comparable conditions).

In some embodiments, the disease or condition is associated with aberrant expression of a YAP target gene. In some embodiments, the disease or condition is associated with aberrant YAP transcription activity.

In some embodiments, the disease or condition is cancer. Cancer cells can become highly dependent on transcription of certain genes, as in transcriptional addiction, and this transcription can be dependent upon specific condensates. For example, a transcriptional SRC-1 condensate might be formed at an oncogene on which the tumor is dependent and this condensate might be specifically targeted by a SRC-1 condensate inhibitor.

In some embodiment, the cancer is a solid tumor or a hematological malignancy. In some embodiments, the cancer is metastatic.

In some embodiments, the cancer is breast cancer, lung cancer, adrenal cancer, lymphoepithelial neoplasia, adenoid cell carcinoma, lymphoma, acoustic neuroma, acute lymphocytic leukemia, acral lentiginous melanoma, acute myeloid leukemia, acrospiroma, chronic lymphocytic leukemia, acute eosinophilic leukemia, liver cancer, acute erythrocyte leukemia, small cell lung cancer, acute lymphocytic leukemia, non-small cell lung cancer, acute megakaryoblastic leukemia, MALT lymphoma, acute monocytic leukemia, malignant fibrous histiocytoma, acute promyelocytic leukemia, malignant peripheral schwannomas, mantle cell lymphoma, adenocarcinoma, marginal zone B-cell lymphoma, malignant hippocampal tumor, adenoid cystic carcinoma, gland tumor, adenoma-like odontogenic tumor, mast cell leukemia, adenosquamous carcinoma, mediastinal germ cell tumor, adipose tissue tumor, breast medullary carcinoma, adrenocortical carcinoma, medullary thyroid carcinoma, adult T cell leukemia/lymphoma, Medulloblastoma, invasive NK cell leukemia, melanoma, AIDS-related lymphoma, meningioma, lung rhabdomyosarcoma, Merkel cell carcinoma, alveolar soft tissue sarcoma, mesothelioma, ameloblastoma, metastatic urothelial carcinoma, anaplastic large cell lymphoma, mixed Müllerian tumor, thyroid undifferentiated carcinoma, mucinous neoplasm, angioimmunoblastic T-cell lymphoma, multiple myeloma, angiomyolipoma, muscle tissue tumor, angiosarcoma, mycosis fungoides, astrocytoma, myxoid liposarcoma, atypical deformed rhabdoid tumor, myxoma, B-cell chronic lymphocytic leukemia, mucinous sarcoma, B-cell lymphoblastic leukemia, nasopharyngeal carcinoma, B-cell lymphoma, schwannomas, basal cell carcinoma, neuroblastoma, biliary tract cancer, neurofibromatosis, bladder cancer, neuroma, blastoma, nodular melanoma, bone cancer, eye cancer, Brenner tumor, oligodendroxoma, brown tumor, oligodendroglioma, Burkitt's lymphoma, eosinophilic breast cancer, brain cancer, optic nerve tumor cancer, oral cancer carcinoma in situ, osteosarcoma, carcinosarcoma, ovarian cancer, cartilage tumor, pulmonary sulcus tumor, papillary thyroid carcinoma, myeloma, paraganglioma, chondroma, pineal blastoma, chordoma, pineal cell tumor, choriocarcinoma, pituitary tumor, choroid plexus papilloma, pituitary adenoma, kidney clear cell sarcoma, pituitary tumor, craniopharyngioma, plasmacytoma, cutaneous T-cell lymphoma, multiple embryonic cell tumor, cervical cancer, precursor T lymphoblastic lymphoma, colorectal cancer, primary central nervous system lymphoma, Degos disease, primary effusion lymphoma, proliferative small round cell tumor, primary preformed peritoneal cancer, diffuse large B-cell lymphoma, prostate cancer, embryonic dysplasia of neuroepithelial neoplasia, pancreatic cancer, anaplastic cell tumor, pharyngeal carcinoma, embryonic carcinoma, peritoneal pseudomyxoma, endocrine gland tumor, renal cell carcinoma, enteropathy-associated T-cell lymphoma, endodermal sinus tumor, renal medullary carcinoma, retinoblastoma, esophageal cancer, rhabdomyosarcoma, endadelphos, rhabdomyosarcoma, fibroids, Richter's transformation, fibrosarcoma, rectal cancer, follicular lymphoma, sarcoma, follicular thyroid cancer, schwannoma, ganglion cell tumor, seminoma, gastrointestinal cancer, Sertoli cell tum, germ cell tumor, sex cord-gonadal stromal tumor, pregnancy choriocarcinoma, signet ring cell carcinoma, giant cell fibroblastoma, skin cancer, bone giant cell tumor of bone, small blue round cell tumor, glioma, small cell carcinoma, glioblastoma multiforme, soft tissue sarcoma, glioma, somatostatin tumor, glioma brain, soot wart, pancreatic high glucagonoma, spinal tumor, Gonadoblastoma, spleen marginal lymphoma, granulosa cell tumor, squamous cell carcinoma, estrogen tumor, synovial sarcoma, gallbladder cancer, Sezary disease, gastric cancer, small intestine cancer, hairy cell leukemia, squamous cell carcinoma, hemangioblastoma, gastric cancer, head and neck cancer, T-cell lymphoma, vascular epithelioma, testicular cancer, hematological malignancies, sarcoma, hepatoblastoma, thyroid cancer, hepatosplenic T-cell lymphoma, transitional cell carcinoma, Hodgkin's lymphoma, laryngeal cancer, non-Hodgkin's lymphoma, urachal cancer, invasive lobular carcinoma, genitourinary cancer, intestinal cancer, urothelial carcinoma, renal cancer, uveal melanoma, laryngeal cancer, uterine cancer, malignant freckle-like sputum, verrucous carcinoma, lethal midline granuloma, visual pathway glioma, leukemia, vulvar cancer, testicular stromal tumor, vaginal cancer, liposarcoma, Waldenstrom's macroglobulinemia Disease, adenolymphoma, lymphangioma, nephroblastoma, lymphangisarcoma

In some embodiments, the cancer is breast cancer, lung cancer (optionally non-small cell lung cancer), uveal melanoma, liver cancer, head neck cancer and squamous carcinoma, mesothelioma, or malignant pleural mesothelioma.

In certain embodiments, the pharmaceutically effective amount is sufficient to prevent, alleviate or ameliorate symptoms of a disease or to prolong the survival of the subject being treated. Determination of a pharmaceutically effective amount is well within the capability of those skilled in the art. The pharmaceutically effective amount is varied according to the particular treatment involved for a subject and depend upon various factors known in the art, such as the subject's body weight, size, and health; the nature and extent of the condition; the rate of administration; the therapeutic or combination of therapeutics selected for administration; and the discretion of the prescribing physician. Pharmaceutically effective amounts for a given situation can be determined by routine experimentation that is within the skill and judgment of the clinician. For example, the initial administration dosage may be higher than subsequent administration dosages. For another example, the administration dosage may vary over the course of treatment depending on the reaction of the subject.

Methods of Screening

In another aspect, the present disclosure provides a method of screening for an agent that modulates a SRC-1 condensate, comprising:
a) providing the SRC-1 condensate and assessing one or more physical properties or one or more biological effects of the condensate,
b) contacting the SRC-1 condensate with a test agent, and
c) assessing whether the test agent causes a change in the one or more physical properties or one or more biological effects of the SRC-1 condensate.

In some embodiments, the test agent is identified as modulating the condensate if it causes a change in the one or more physical properties or one or more biological effects of the SRC-1 condensate.

In some embodiments, the SRC-1 condensate is a transcriptional SRC-1 condensate. In some embodiments, the transcriptional SRC-1 condensate further comprises a first component capable of interacting with SRC-1. In some embodiments, the first component comprises Yes-associated protein (YAP), Estrogen receptor(ER), androgen receptor (AR), vitamin D receptor (VDR) and AP-1. In some embodiments, the transcriptional condensate further comprises a second component interacting with the first component. In some embodiments, the second component comprises a TEA-domain transcription factor. In some embodiments, the TEA-domain transcription factor comprises TEAD1, TEAD2, TEAD3, TEAD4 or any combination thereof. In some embodiments, the transcriptional condensate further comprises a RNA polymerase. In some embodiments, the transcriptional condensate further comprise a histone.

In some embodiments, the SRC-1 condensate is inside a cell or inside nucleus. The condensate may be a naturally occurring condensate. In other embodiments, the condensate may occur in a transgenic cell or an otherwise manipulated cell. In some embodiments, the method of screening is performed in a cell-based system, comprising providing a cell having a SRC-1condensate, contacting the cell with a test agent, and determining if contact with the test agent causes a change in the one or more physical properties or one or more biological effects of the SRC-1 condensate.

In some embodiments, the method of screening is performed in a cell-free system, comprising provide an isolated cellular composition comprising a SRC-1 condensate, contacting the composition with a test agent, and determining if contact with the test agent causes a change in the one or more physical properties or one or more biological effects of the SRC-1 condensate. In some embodiments, the isolated cellular composition comprising a nucleus comprising a SRC-1 condensate.

The type of cell having a condensate or from which a composition having a SRC-1 condensate is isolated is not limited and may be any cell type disclosed herein. In some embodiments, the cell is affected by a disease (e.g., a cancer cell). In some embodiments, the cell having a condensate is a primary cell, a member of a cell line, cell isolated from a subject suffering from a disease, or a cell derived from a cell isolated from a subject suffering from a disease (e.g., a progenitor of an induced pluripotent cell isolated from a subject suffering from a disease).

In some embodiments, the SRC-1 condensate is a synthetic condensate (also see description below). A synthetic condensate can appear as a liquid droplets in vitro composed of SRC-1. In some embodiments, the synthetic condensate further comprises one or more components (e.g., the first and second components as described herein). Such droplets may further comprise RNA, DNA and/or histones. Such liquid droplets are in vitro condensates and can correspond to and/or serve as models of condensates that exist in vivo.

In some embodiment, the physical properties of a SRC-1 condensate is measured. Physical properties can include, without limitation, composition, stability, size, concentration, permeability, morphology and viscosity. Any suitable method known in the art may be used to measure the one or more physical properties. These physical properties can correlate with the condensate's ability to activate a reporter gene.

In some embodiments, the method comprising providing a cell, an isolated cellular composition comprising a SRC-1 condensate, or a synthetic SRC-1 condensate and assessing one or more physical properties of the condensate, contacting the condensate with a test agent, and assessing whether the test agent causes a change in the one or more physical properties of the condensate. In some embodiments, an agent identified as causing one or more physical properties of the condensate is further tested to assess its effect on one or more functional properties (i.e., biological effects) of a condensate, e.g., ability to modulate transcription of one or more genes associated with the condensate

In some embodiments, the condensate has a detectable tag and the detectable tag is used to determine if contact with the test agent causes any changes in the one or more physical properties or one or more biological effects of the SRC-1 condensate. In some embodiments, the cell is a genetically engineered to express the detectable tag. The term “detectable tag” as used herein includes, but is not limited to, detectable labels, such as fluorophores, radioisotopes, colorimetric substrates, or enzymes; heterologous epitopes for which specific antibodies are commercially available, e.g., FLAG-tag; heterologous amino acid sequences that are ligands for commercially available binding proteins, e.g., Strep-tag, biotin; fluorescence quenchers typically used in conjunction with a fluorescent tag on the other polypeptide; and complementary bioluminescent or fluorescent polypeptide fragments. A tag that is a detectable label or a complementary bioluminescent or fluorescent polypeptide fragment may be measured directly (e.g., by measuring fluorescence or radioactivity of, or incubating with an appropriate substrate or enzyme to produce a spectrophotometrically detectable color change for the associated polypeptides as compared to the unassociated polypeptides). A tag that is a heterologous epitope or ligand is typically detected with an additional agent that binds thereto, e.g., an antibody or binding protein, wherein the agent is associated with a detectable label. In some embodiments, SRC-1 or a condensate component (e.g., the first or second component as described herein) comprises a detectable label.

In some embodiments, the test agent is assessed to determine whether one or more of the following physical properties of a SRC-1 condensate is change upon the contact (i) number of SRC-1 condensates; (ii) size of SRC-1 condensates; (iii) location of SRC-1 condensates; (iv) distribution of SRC-1 condensates. (v) surface area of SRC-1 condensates; (vi) composition of SRC-1 condensates; (vii) liquidity of SRC-1 condensates; (viii) solidification of SRC-1 condensates; (ix) dissolution of SRC-1 condensate

Any suitable method of detecting changes of the condensate upon the contacting of the test agent may be used, including methods known in the art and taught herein. In some embodiments, the step of determining if contact with the test agent causes changes to properties of a condensate is performed using microscopy. In some embodiments, the microscopy is deconvolution microscopy, structured illumination microscopy, or interference microscopy. In some embodiments, the step of determining if contact with the test agent causes changes to properties of a condensate is performed using DNA-FISH, RNA-FISH, or a combination thereof.

In some embodiments, one or more biological effects of the transcriptional SRC-1 condensate is assessed based on expression of a target gene in a condensate-dependent manner. In some embodiments, the target gene is a reporter gene. Such reporter gene can be operatively linked to a binding site for YAP/TEAD, AR, EP VDP or AP-1. In some embodiments, the reporter gene encode a fluorescent or luminescent protein that are detectable. In some embodiments, the target gene is a YAP target gene. In some embodiment, the target gene is a ANKRD1, CTGF or CYP61

In some embodiments, the SRC-1 condensate drives the expression of the reporter gene prior to contact with a test agent, and stops or reduces the expression of the reporter gene after contact with an agent that suppresses, degrades, or prevents condensate formation, stability, function, or morphology.

In some embodiments, one of more biological effects of the transcriptional SRC-1 condensate is assessed based on the proliferation of SRC-1 condensate-containing cells. Proliferation of cells can be assessed by any suitable methods that are described herein or known in the art, e.g., a cell viability assay. In some embodiments, the SRC-1 condensate-containing cells are tumor cells. In some embodiments, the proliferation of the SRC-1 condensate-containing cells are reduced after contact of a test agent with the cells.

In some embodiments, one of more biological effects of the transcriptional SRC-1 condensate is assessed based on the acetylation of histones. The acetylation of histones can be assessed by any suitable methods that are described herein or known in the art, e.g., ChIP-qPCR or immunofluorescence imaging. In some embodiments, the acetylation of histones is increased upon after contact of a test agent with the SRC-1 condensate.

In another aspect, the present disclosure provides a method of identifying an agent that modulates formation of a SRC-1 condensate, comprising:
a. providing components capable of forming the SRC-1 condensate;
b. contacting the components with a test agent under the condition suitable for formation of the SRC-1 condensate, and
c. assessing whether presence of the test agent affects formation of the SRC-1 condensate or one or more biological effects of the SRC-1 condensate.

In some embodiments, the test agent is identified as modulating the formation of the condensate if it affects formation of the condensate or affects the one or more biological effects of the SRC-1 condensate.

For example, one can provide the components (e.g., SRC-1 and the first or second component as described herein), combine them in a vessel, and observe what happens in terms of condensate formation and/or measure the properties (e.g., increases or decreases in stability, composition, activity, morphology) of resulting condensates. In some embodiments, the provided composition will form a condensate and the test agent will be screened for modulating formation (e.g., increasing or decreasing condensate formation or the rate of condensate formation).

In some embodiments, a method of screening an agent that modulates a SRC-1 condensate formation comprises providing a cell, an isolated cellular composition and/or an in vitro transcription assay expressing a reporter gene under the control of a SRC-1 condensate, contacting the cell or assay with a test agent, and assessing expression of the reporter gene.

In some embodiments, the method may be performed to identify an agent that interacts with SRC-1 and drives SRC-1 into a transcriptional condensate. In some embodiments, the method may be performed to identify an agent that interacts with SRC-1 and prevents integration of SRC-1 into a condensate. In some embodiments, the method may be performed to identify an agent that force integration of a component into a SRC-1 condensate or prevent a component from entering a SRC-1 condensate. In some embodiments, an agent identified by the methods disclosed herein of modulating a SRC-1 condensate or the formation of a SRC-1 condensate is further tested for its ability to modulate one or more features of a disease. The disease is not limited and may be any disease disclosed herein. For example, if the agent inhibits the expression of a reporter gene or the formation of a SRC-1 condensate, could test the ability of the agent to inhibit proliferation of cancer cells that has an elevated expression of SRC-1 relative to a reference.

In some embodiments, an agent identified as modulating one or more physical properties or formation of a condensate (e.g., formation, stability, or morphology) or functional properties of a condensate (e.g. modulation of transcription) by the methods disclosed herein may be administered to a subject, e.g., a non-human animal that serves as a model for a disease, or a subject in need of treatment for the disease.

In some embodiments, a high throughput screen (HTS) is performed. A high throughput screen can utilize either cell-free or cell-based assays (e.g., a condensate containing cell, a synthetic condensate, an isolated cellular composition). High throughput screens often involve testing large numbers of test agents with high efficiency, e.g., in parallel. For example, tens or hundreds of thousands of compounds can be routinely screened in short periods of time, e.g., hours to days. Often such screening is performed in multiwell plates containing, at least 96 wells or other vessels in which multiple physically separated cavities or depressions are present in a substrate. High throughput screens often involve use of automation, e.g., for liquid handling, imaging, data acquisition and processing, etc. Certain general principles and techniques that may be applied in embodiments of a HTS of the present invention are described in Macarron R & Hertzberg RP. Design and implementation of high-throughput screening assays. Methods Mol Biol., 565:1-32, 2009 and/or An WF & Tolliday NJ., Introduction: cell-based assays for high-throughput screening. Methods Mol Biol. 486:1-12, 2009, and/or references in either of these.

In some embodiments, an analog of an agent identified as modulating one or more physical properties or formation of a condensate (e.g., formation, stability, function, or morphology) or functional properties of a condensate (e.g. modulation of transcription) by the methods disclosed herein may be generated. An “analog” of a first agent refers to a second agent that is structurally and/or functionally similar to the first agent. An analog of an agent may have substantially similar physical, chemical, biological, and/or pharmacological propert(ies) as the agent or may differ in at least one physical, chemical, biological, or pharmacological property. In some embodiments at least one such property differs in a manner that renders the analog more suitable for a purpose of interest, e.g., for modulating a condensate. Methods of generating analogs are known in the art and include methods described herein. In some embodiments, generated analogs can be tested for a property of interest, such as increased stability (e.g., in an aqueous medium, in human blood, in the GI tract, etc.), increased bioavailability, increased half-life upon administration to a subject, increased cell uptake, increased activity to modulate a condensate property including physical properties or formation of a condensate (e.g., formation, stability, function, or morphology) or functional properties of a condensate (e.g. modulation of transcription), increased specificity for a condensate.

Synthetic Condensate

In another aspect, the present disclosure provides a synthetic SRC-1 condensate comprising at least SRC-1 or a fragment thereof comprising an intrinsic disorder domain of SRC-1. As used herein, a “synthetic” condensate refers to a non-naturally occurring condensate comprising condensate components. In some embodiment, the synthetic SRC-1 condensate is a synthetic transcriptional SRC-1 condensate. In some embodiments, the synthetic SRC-1 condensate simulates a transcriptional SRC-1 condensate found in a cell.

The synthetic SRC-1 condensates may comprise any of the components described herein. In some embodiments, the condensate comprises a first component (e.g., YAP, ER, AR, VDR or AP-1) capable of interacting with SRC-1. In some embodiments, the condensate comprises a second component (e.g., TEAD1, TEAD2, TEAD3, TEAD4) capable of interacting with the first component. In some embodiments, the condensate comprises a RNA polymerase (e.g., Pol II). In some embodiment, the condensate comprises an isolated polynucleotide (e.g., a reporter gene).

In some embodiments, the synthetic SRC-1 condensate comprises SRC-1 or a fragment thereof comprising an IDD. In some embodiments, the fragment of SRC-1 can form or incorporate into a condensate under relevant physiological conditions (e.g., conditions the same as or approximating conditions in a cell) or relevant experimental conditions (e.g., suitable conditions for the formation of a condensate in vitro). In some embodiments, the fragment of SRC-1 can interact with or bind YAP.

In some embodiments, the fragment is fused with an inducible oligomerization domain. In some embodiments, the domain that confers inducible oligomerization is inducible with a small molecule, protein, or nucleic acid. In some embodiments, SRC-1 or a fragment thereof further comprises a detectable tag as described herein. In some aspects, the detectable tag is a fluorescent tag.

Some aspects of the disclosure provide methods of making synthetic transcriptional condensates. In some embodiments the method comprises combining two or more condensate components in vitro under conditions suitable for formation of transcriptional condensates. The conditions can include appropriate concentrations of components, salt concentration, pH, etc. In some embodiments, the conditions include a salt concentration (e.g., NaCl) of about 25 mM, 40 mM, 50 mM, 125 mM, 200 mM, 350 mM, or 425 mM; or in the range of about 10-250 mM, 25-150 mM, or 40-100 mM. In some embodiments, the conditions include a pH of about 7-8, 7.2-7.8, 7.3-7.7, 7.4-7.6, or about 7.5.

In Vitro Screening System

In another aspect, the present disclosure provides an in vitro screening system comprising SRC-1 or a fragment thereof comprising an intrinsic disorder domain of SRC-1, and a detectable label, wherein the SRC-1 or the fragment thereof is capable of forming SRC-1 condensate. In some embodiment, the SRC-1 condensate is a transcriptional condensate.

In some embodiment, in vitro screening system is based on SRC-1 condensates in a cell. The cell can be a transgenic cell or otherwise manipulated cell. In some embodiments, in vitro screening system is based on in vitro SRC-1 condensates. In some embodiments, the in vitro SRC-1 condensate comprises components mimicking a condensate found in a cell.

In some embodiments, in vitro SRC-1 condensates are synthetic condensates with one or more condensate components in a solution. In some embodiments, an in vitro SRC-1 condensate is isolated from a cell. Any suitable means of isolation of a condensate from a cell or composition is encompassed herein (e.g., chemically or immunologically precipitated). In some embodiments, a condensate is isolated by centrifugation (e.g., at about 5,000×g, 10,000×g, 15,000×g for about 5-15 minutes; about 10,000×g for about 10 min). A condensate may be isolated from a cell by lysis of the nucleus of a cell with a homogenizer (i.e., Dounce homogenizer) under suitable buffer conditions, followed by centrifugation and/or filtration to separate the condensate.

In some embodiments, the detectable label is attached to the SRC-1 or the fragment thereof. In some embodiments, the detectable label comprises a fluorophore, a radioisotope, a colorimetric substrate, or an antigenic epitope. In some embodiments, the in vitro condensate comprises a plurality of detectable tags as described herein. In some embodiments, different detectable tag are attached to SRC-1 and different components of SRC-1 (e.g., SRC-1 or a fragment thereof labeled with one fluorescent tag, and YAP or Pol II labeled with a different fluorescent tag). In some embodiments, one or more components of the condensate have a quencher.

In some embodiments, the in vitro screening system further comprises a first component capable of interacting with SRC-1. In some embodiments, the first component comprises Yes-associated protein (YAP), Estrogen receptor(ER), androgen receptor(AR), vitamin D receptor(VDR) and AP-1. In some embodiments, the in vitro screening system further comprises a second component interacting with the first component. In some embodiments, the second component comprises a TEA-domain transcription factor. In some embodiments, the TEA-domain transcription factor comprises TEAD1, TEAD2, TEAD3, TEAD4 or any combination thereof. In some embodiments, the in vitro screening system further comprises a RNA polymerase. In some embodiments, the in vitro screening system further comprises a cell lysate or a nuclear lysate.

Modified Host Cell

In another aspect, the present disclosure provides a modified host cell expressing SRC-1 or a fragment thereof comprising an intrinsic disorder domain, wherein the SRC-1 or the fragment is capable of forming SRC-1 condensate, and wherein the host cell further comprises a detectable label that allows for detection of the SRC-1 condensate. “Host cell” as used herein refers to a eukaryotic cell to which an expression vector encoding an exogenous protein or peptide is introduced, as well a any progeny or potential progeny of such a cell. Because certain modifications may occur in succeeding generations due to either mutation or environmental influences, such progeny may not, in fact, be identical to the parent cell, but are still included within the scope of the term as used herein.

In some embodiments, the detectable label is attached to the SRC-1 or the fragment thereof. In some embodiments, the detectable label comprises a fluorophore, a radioisotope, a colorimetric substrate, or an antigenic epitope.

In some embodiments, the modified host cell further comprises a YAP-responsive reporter construct. In some embodiments, the YAP-responsive reporter construct comprises a reporter gene operably linked to a promoter responsive to YAP activity.

In some embodiments, the host cell is a tumor cell.

In another aspect, the present disclosure provides a modified host cell expressing: a) SRC-1 or a fragment thereof comprising an intrinsic disorder domain thereof, and b) YAP or a functional equivalent thereof, wherein the host cell comprises a YAP-responsive reporter construct.

In another aspect, the present disclosure provides a method of screening for an agent that inhibits SRC-1, comprising:
c. contacting a test agent with the modified host cell provided herein under a condition suitable for expression of the reporter gene;
d. assessing change in expression of the reporter gene in response to the test agent; wherein the change in expression of the reporter gene indicates inhibition of SRC-1.

The modified host cell as disclosed herein can be used to produce the in vitro screening system. In one embodiment, the host cell is cultured (into which a recombinant expression vector encoding a SRC-1 or a fragment fused to a detectable tag has been introduced) in a suitable medium until SRC-1 or a fragment thereof is produced, and then a composition comprising a SRC-1 condensate is isolated from the cell.

The modified host cells of the invention can also be used to produce nonhuman transgenic animals. The nonhuman transgenic animals can be used in screening assays designed to identify agents which are capable of modulating SRC-1 condensate and ameliorating detrimental symptoms of cancer.

### EXAMPLES

While the disclosure has been particularly shown and described with reference to specific embodiments (some of which are preferred embodiments), it should be understood by those having skill in the art that various changes in form and detail may be made therein without departing from the spirit and scope of the present disclosure as disclosed herein.

The methods and materials used in the examples are described in general below.

Methods and Materials

i. Cell Culture

H1299, H1838, MCF7, A549, HT29, RKO cell lines purchased from the Cell Bank, Chinese Academy of Sciences, have been authenticated by STR analysis and mycoplasma detection. SF268 was kindly provided by Zhang Wei-Min lab. Beas-2B were obtained from JNJ, OCM-1/OCM-8 were gifts from Yu Faxin Lab, 293FT was a gift from Wang Wenyuan lab. These cells have been verified through periodic morphology checks and mycoplasma detection.

Cells were maintained accordingly to the guidance from American Type Culture Collection (DMEM/RPMI1640 supplemented with 10% (v/v) FBS (Gibco), 100 units/mL penicillin and 100 mg/mL streptomycin (Gibco)). All cells were cultured at 37° C. in a humidified atmosphere of 95% air and 5% CO2.

ii. Plasmids/siRNAs Transfection and Viral Infection

Transfection of plasmids into SF268/H1299/A549/BEAS-2B was performed using Lipofectamine3000 (Life Technologies) according to the manufacturer's instructions. Transfection of plasmids into HEK293T/293FT was performed using Polyjet (SL100688, SignaGen) according to the manufacturer's instructions. Transfection of siRNAs was performed using Lipofectamine RNAi MAX (Life Technologies) according to the manufacturer's instructions.

To generate stable cells, lentiviral or retroviral infections were used. Briefly, HEK293FT cells were co-transfected with viral plasmids and packaging plasmids. Forty-eight hours after transfection, culture medium was filtered through a 0.45 um filter, and used to infect cells of interest.

iii. Live Cell Imaging

Cells were grown on 24-well glass bottom plate (Cellvis, P24-1.5H-N) and images were taken with the Leica TCS SP8 confocal microscopy system using a 100× oil objective (NA=1.4). Cells were imaged on a heated stage(37° C.) and supplemented with warmed (37° C.) humidified air.

For compounds treatment assay, SF268 cells were co-transfected with YAP5SA, TEAD4-mTagBFP2 and mNeoGreen-SRC1. YAP variant YAP5SA which is insensitive to the upstream Hippo pathway, sequestered YAP in nucleus. To circumvent the limitations brought by red fluorescent proteins, TEAD condensates which have been reported to depend on YAP, were used to characterize YAP/TEAD transcriptional condensates. Six hours after transfection, SF268 cells were seeded on 24-well glass bottom plate (Cellvis, P24-1.5H-N) and compounds were added two hours later. Cells were imaged every 30 mins after EVG or other compounds were added.

Fluorescent images were processed and assembled into figures using LAS X (Leica) and Fiji.

iv. High Content Image

Eight hours after transfection, H1299 cells expressing mScarlet-SRC-1 were seeded in 24-well glass bottom plate (Cellvis P24-1.5H-N) and EVG was added two hours later. Images were obtained with the Operetta CLSTM high-content cell imaging analysis system (PerkinElmer Inc., Waltham, MA, USA). The 63× objective lens was applied in each condition. Data was analyzed by images collected from over 100 representative fields in each group. The spots quantification was performed based on the area and intensity of the spots through mScarlet channel. GraphPad Prism is used to plot and analyze the high content image results.

v. Fluorescence Recovery after Photobleaching (FRAP)

FRAP assay was conducted using the FRAP module of the Leica SP8 confocal microscopy system. The TEAD4-mTagBFP, mClover3-YAP and mScarlet-SRC1 were bleached using a 405/488/561-nm laser beam, respectively. Bleaching was focused on a circular region of interest (ROI) using 100% laser power and time-lapse images were collected. Fluorescence intensity was measured using FIJI. Background intensity was subtracted and values are reported relative to pre-bleaching time points. GraphPad Prism is used to plot and analyze the FRAP results.

vi. In Vitro Liquid-Liquid Phase Separation (LLPS) Assay

For the LLPS assay, the purified SRC1(final 50 μM) was mixed with a LLPS buffer containing 20 mM Tris pH8.0, 10% (w/v) PEG8000 (Sigma) and incubated for 10 min at room temperature. Finally, 2 μL of each sample was pipetted onto a glass dish and imaged using a Leica microscope.

vii. Immunofluorescence

Cells were fixed with 4% PFA for 15 minutes at room temperature. Cells were subsequently washed with PBS (3×3 min) and blocked in PBS/BSA (3%)/Triton X-100 (0.3%) at room temperature for 60 minutes. Cells were rinsed in PBS and incubated with anti-H3K27ac (ab4729, Abcam, 1:100), anti-RNA Pol II-S5P antibody (#04-1572, Millipore, 1:200) in PBS/BSA (1%) overnight at 4° C. Cells were washed with PBST (1×PBS+Tween20 0.2%, 3×5 min) and incubated with the secondary antibody for 1 hours at room temperature. After washing with PBST(3×5 min), cells were stained with DAPI.

viii. Protein Expression and Purification

YAP, TEAD4, and SRC1 truncations were expressed as His fusions in E. coli BL21 (DE3). Cells were grown to an OD of 0.6-0.8, then isopropyl 1-thio-β-d-galactopyranoside (IPTG) was added to induce protein expression, followed by incubation overnight at 16° C. Cell pellets were collected and sonicated in lysis buffer (20 mM Tris-HCl pH 8.0, 150 mM NaCl, 1 mM PMSF). Then supernatants were centrifuged and incubated with Ni Sepharose 6 Fast Flow (17-5318-01, GE Healthcare). The resin was washed, and the protein was eluted using lysis buffer supplemented with 50-500 mM imidazole.

ix. Luciferase Reporter Assays

YAP binding sites were cloned into the NheI and HindIII sites of the pGL4.76 basic vector (Promega) containing heat shock basal promoter. Luciferase activity was measured using the Renilla-Glo® Luciferase Assay System (Promega). Cell viability was measured using CellTiter-Glo® One Solution Assay (Promega). Relative luciferase activity=luciferase activity/cell viability.

x. Co-Immunoprecipitation

CoIP was performed as described in the protocols of Dynabeads™ Protein G for Immunoprecipitation Kit (10003D, Invitrogen). Whole cell lysates from SF268 were prepared in CoIP lysis buffer (20 mM Tris-HCl 7.5, 150 mM NaCl, 0.5% NP40, 10% glycerin, 1% proteinase Cocktail). Supernatants were collected by centrifugation, and incubated with anti-SRC1 antibody or anti-TEAD4 antibody or IgG coupled to Dynabeads™ Protein G. The beads were washed in lysis buffer (4×5 min) and samples were examined by immunoblotting.

xi. FLAG Pulldown Assay

Three independent pull down assays were performed to detect the direct binding between SRC1 and YAP/TEAD4. Purified FLAG-SRC1 truncated proteins were incubated with YAP, TEAD4 and YAP plus TEAD4 respectively, and further subjected to pull down assays by ANTI-FLAG® M2 Affinity Gel (A2220, Sigma-Aldrich). Elvitegravir was co-incubated with the mixtures at increasing concentrations (0, 50, 200, 400 μM) as indicated. Complexes were washed in PBS buffer and proteins were eluted using 2×SDS loading buffer.

xii. RT-PCR

Cells were treated with compounds for indicated periods, then total RNAs were extracted with TRIzol™ Reagent (15596018, Invitrogen) and reverse-transcripted using the High efficient cDNA synthesis master mix (FSQ-301, TOYOBO). Quantitative RT-PCR was performed with 440 SYBR® Premix Ex Taq™ (RR420, Takara). Quantitative real-time PCR was carried out with QuantStudio™ 6 Flex Real-Time PCR Systems (Thermofisher Scientific). qPCR Primers are listed in Supplementary Table S1.

xiii. ChIP-qPCR

Briefly, cells were crosslinked with 1% formaldehyde (Sigma) in culture medium for 10 min at room temperature, and chromatin from lysed nuclei was sheared to 200-1000 bp fragments using a Bioruptor® Pico (Diagenode SA). ˜50 μg of sheared chromatin and ˜2 μg of antibody were mixed. Antibody/antigen complexes were recovered with ProteinG-Dynabeads (Invitrogen) for 1.5 h at 4° C. Quantitative real-time PCR was carried out with QuantStudio™ 6 Flex Real-Time PCR Systems (Thermofisher Scientific). The amount of immunoprecipitated DNA in each sample was determined as the fraction of the input, and normalized to the IgG control. PCR Primers are listed in Supplementary Table S2.

xiv. Analysis of Public ChIP-Seq Data

We used the deepTools module plotHeatmap to analyze the peak calling on ChIP-seq data followed by the protocol reported previously56. The ChIP-seq data of human K562 cell line were download from Cistrome Data Browser (http://cistrome.org/db/#/), including 64089_peaks.bed, Galaxy14-[46284.bw].bigwig, Galaxy6-[64887.bw].bigwig, Galaxy7-[57447.bw].bigwig, Galaxy8-[68375.bw].bigwig, Galaxy9-[57417.bw].bigwig. We used the WashU Brower (http://cistrome.org/db/#/) and IGV to view YAP, TEAD and SRC-1 ChIP enrichment at genome.

xv. RNA-Seq

Total RNA of three biological replicates was extracted from SF268 cells 24 h after treated with 20 μM EVG/DMSO using TRIzol™ Reagent (15596018, Invitrogen). RNAseq libraries were prepared using the Illumina TruSeq RNA Sample Prep kit v2 and sequenced using the Illumina HiSeq Xten platform (150-bp paired-end reads). We mapped the RNA-seq sequencing reads to the human reference transcriptome (GRCh37.71) using HISAT259 version 2.0.1. Differentially expressed genes in elvitegravir-treatment group were identified with the R package DESeq.

xvi. Biolayer Interferometry (BLI)

The binding between EVG and SRC1 was examined using BLI Octet RED96 (ForteEio Inc., Menlo Park, CA). Streptavidin biosensors (ForteBio Inc.) were pre-soaked (30 min) in assay buffer (20 mM TrisHCl pH8.0, 150 mM NaCl and 1% DMSO), then coated in an assay buffer containing 100 uM EVG-biotin for 7 min. As a control, sensors were incubated in an assay buffer containing 100 uM Biocytin, followed by a 6 min wash with assay buffer containing 1% BSA and 0.1% Tween20. Indicated concentrations of truncated SRC1protein (500, 166.7, 55.6, 6.2, 0 μg/ml) were diluted in assay buffer containing 1% BSA/0.1% Tween20 and flowed through biosensors coated with Bio-EVG or biocytin for 5 min. A 5 min dissociation was followed. Data was analyzed using OctetRED analysis software.

xvii. Biotin-EVG Pull Down with Purified Protein

1 μg of purified recombinant protein Flag-SRC-1 was incubated with 2 M biotin-EVG/DMSO in the presence of increasing concentrations of Elvitegravir (20, 40, 100, 200 uM) for 30 min (RT) and was further incubated with streptavidin-agarose (20359, Thermo Fisher Scientific) for 20 min (RT). Washing beads with PBS (3×1.5 min). The precipitates were analyzed by western blot with anti-flag antibody.

xviii. Statistical Analysis

All data are presented as the mean±standard error of mean (s.e.m.) or standard deviation (s.d.) from independent determinations, and statistical analyses were done using the software Graphpad Prism version 6.0 (GraphPad Software, Inc.; La Jolla, CA, USA). Differences of means were tested for statistical significance with two/one-tailed Student's t test.

### Example 1

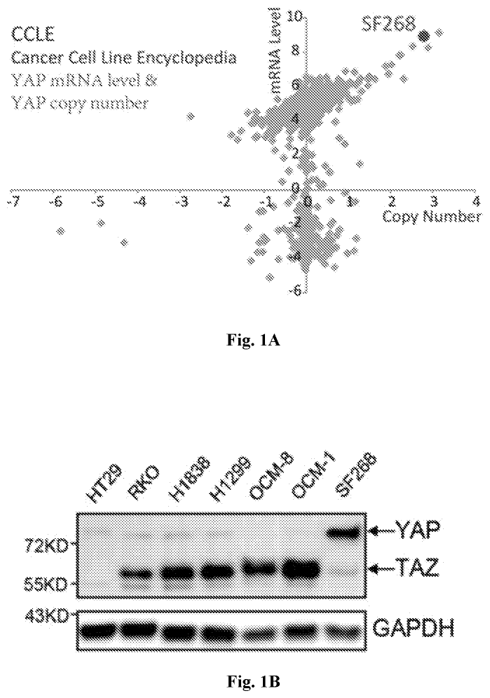

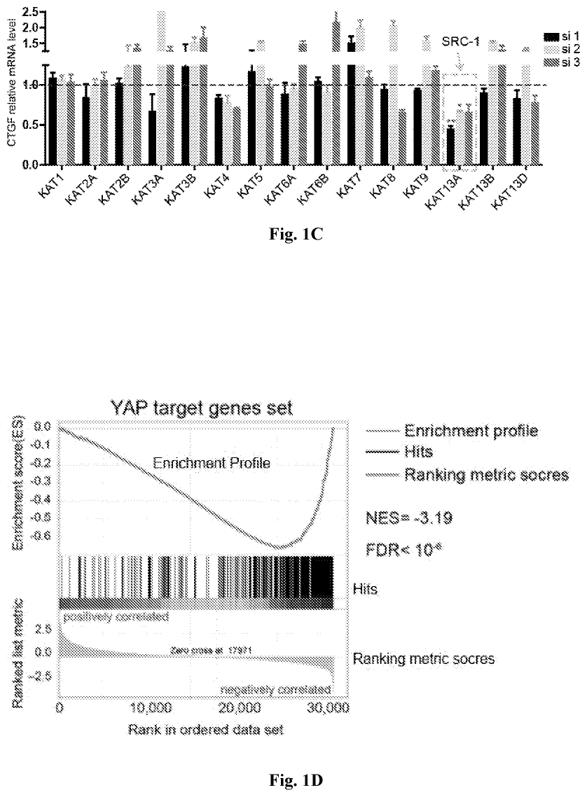

To gain insights into the epigenetic regulatory mechanism of YAP activity, HAT enzymes was explored whether they might be responsible for YAP target genes expression. A focused genetic screen using an siRNA library containing three independent siRNAs for 15 reported HATs encoded by the human genome was performed in SF268 cells which are derived from human glioblastoma harboring YAP gene amplification (13 copies) and expressing high YAP protein levels (FIGS. 1A and 1B). Knockdown of SRC-1 (KAT13A) by all three siRNAs consistently reduced the expression of YAP-targeted CTGF (FIG. 1C) and global gene expression profiles (FIG. 1D) further corroborates the SRC-1 regulation on YAP target genes. These data demonstrate that SRC-1 facilitates YAP transcriptional activity.

### Example 2

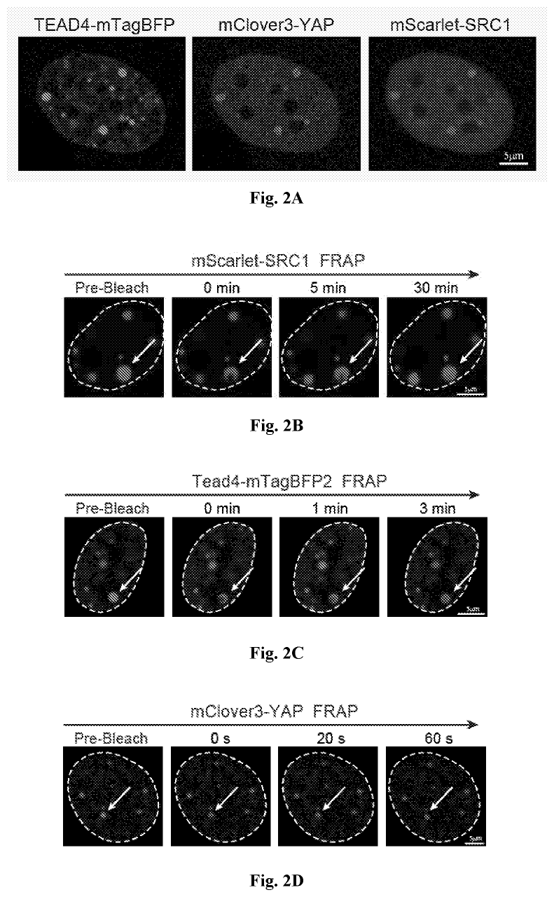

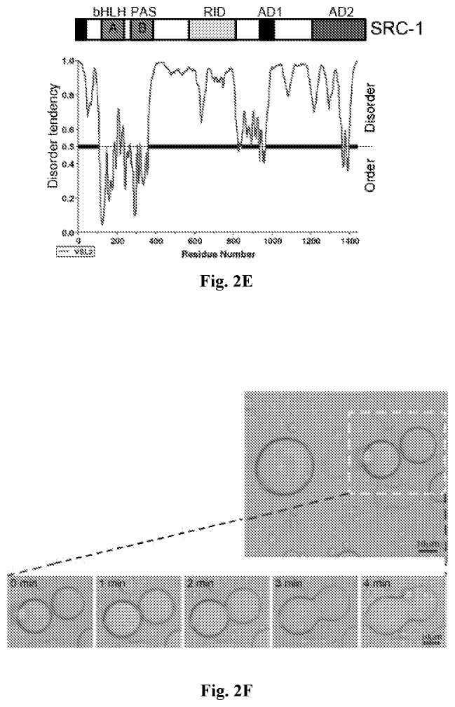

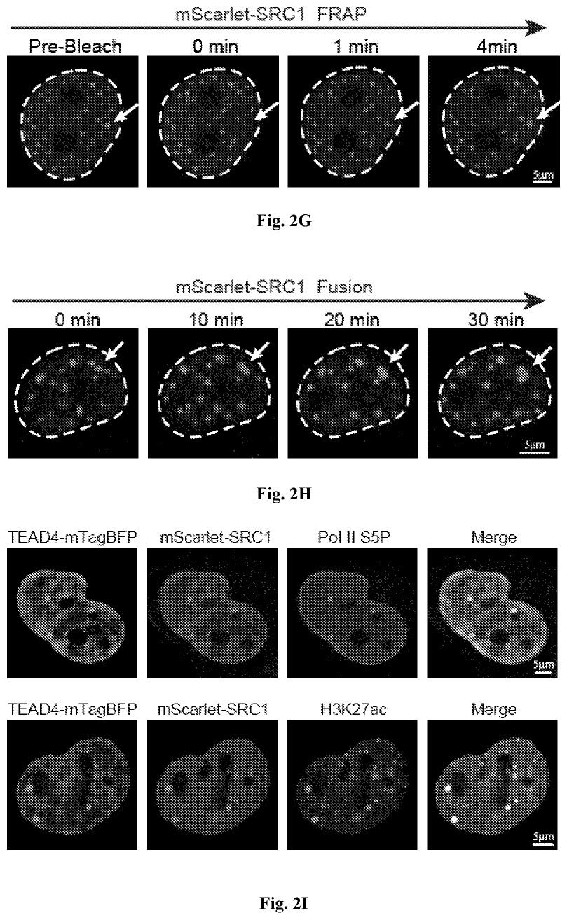

To investigate whether SRC-1 co-exist in YAP/TEAD phase-separated transcriptional condensates, mClover3-YAP, TEAD4-mTagBFP2 and mScarlet-SRC-1 were ectopically expressed in SF268 cells. Microscopic imaging revealed that SRC-1 distributes to the YAP/TEAD condensates (FIG. 2A) and exhibits rapid recovery upon photobleaching (Supplementary information, FIGS. 2B-D). Given that SRC-1 protein contains large IDDs (FIG. 2E), its intrinsic potential to undergo LLPS was investigated and it is found that SRC-1 could form LLPS condensates both in vitro and in cells (FIGS. 2F-H). FRAP and fusion experiments further corroborate its liquid-like properties (FIGS. 2G and 2H). Importantly, both CTD-phosphorylated RNA polymerase II and H3K27ac were enriched in the YAP/TEAD/SRC-1 condensates (FIG. 2I), suggesting that the SRC-1 co-occupied YAP/TEAD LLPS puncta are the sites of active transcription. These results support that SRC-1 forms LLPS condensates compartmented with YAP/TEAD to promote gene expression.

### Example 3

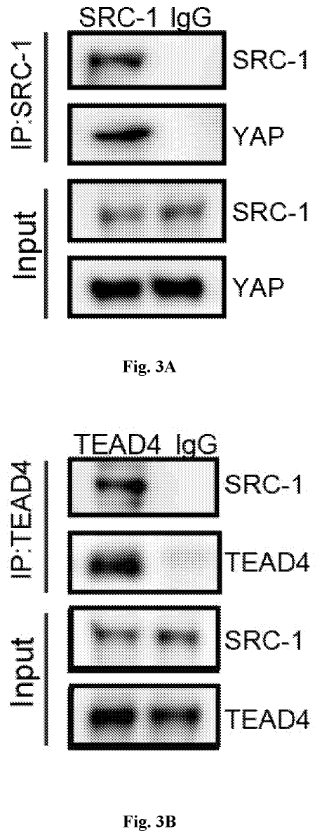

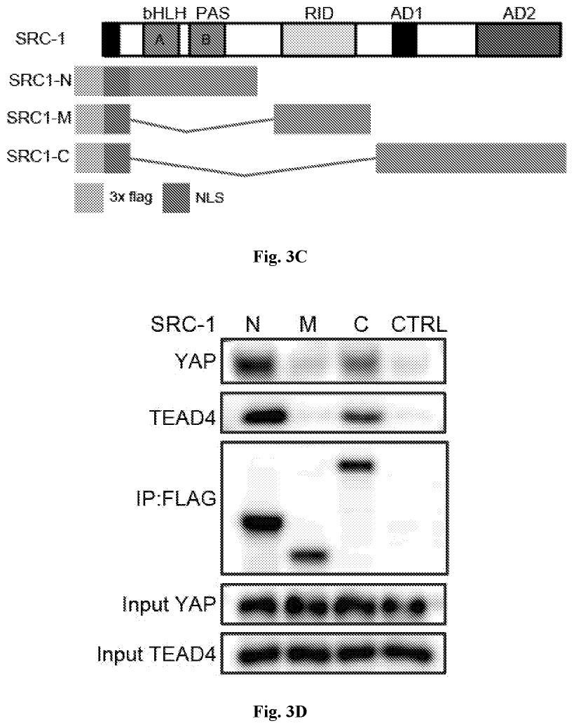

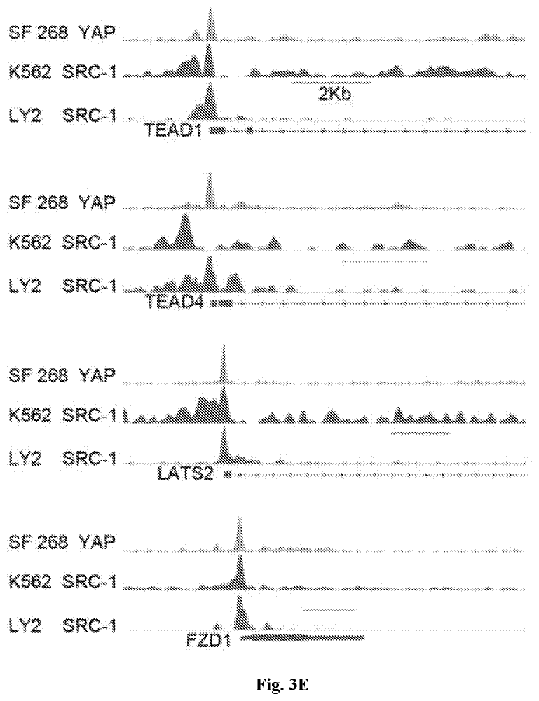

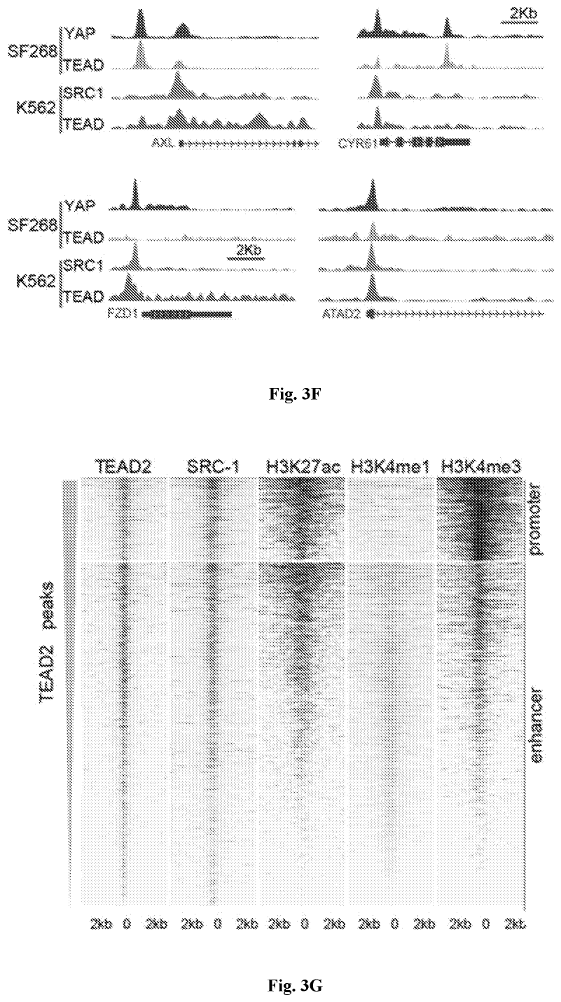

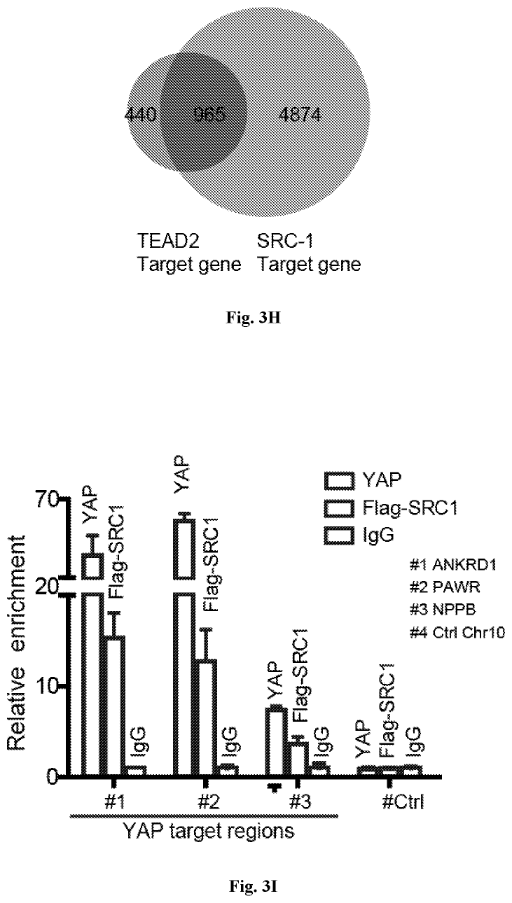

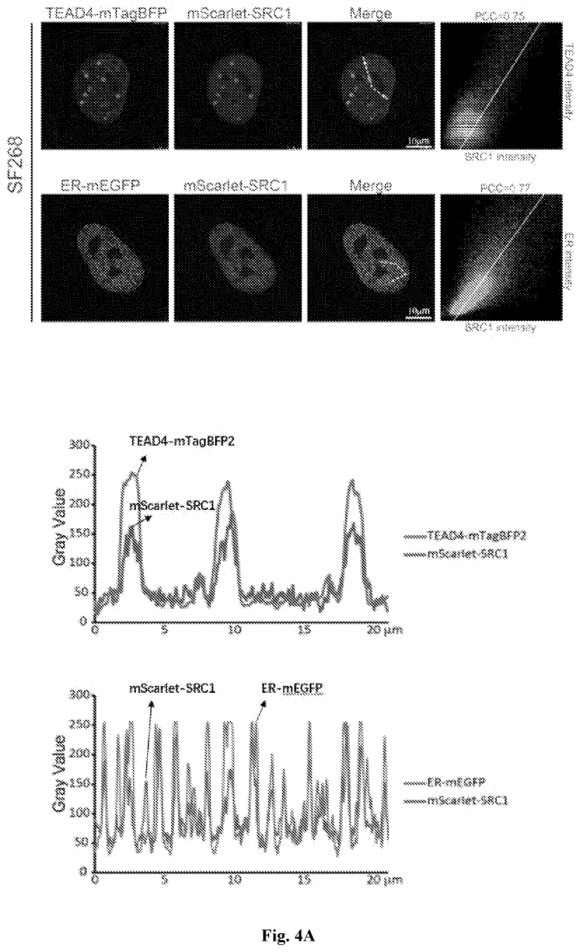

Coimmunoprecipitation experiments were performed under endogenous conditions (FIGS. 3A and 3B) to verify SRC-1 is a component of YAP/TEAD complex. To characterize the domain of SRC-1 that interacts with YAP and TEAD, 293T cells was transfected with truncated forms of SRC-1 and found that both the bHLH-PAS (SRC1-N) and AD (SRC1-C) domains participated in the interactions with YAP and TEAD (FIGS. 3C and 3D). Then previously reported ChIP-seq datasets (Davis, C. A. et al. Nucleic Acids Res. 46, D794-D801 (2018)) was analyzed to further explore the genome-wide regulation of SRC-1 on YAP/TEAD transcriptome. Significant genomic co-occupancy of SRC-1 with both YAP and TEAD2 was observed (FIGS. 3E-H). Consistently, ChIP-qPCR data in SF268 cells confirmed the enrichment of SRC-1 and YAP/TEAD in the loci of YAP target genes including ANKRD1, NBBP and PAWR (FIG. 3I).

### Example 4

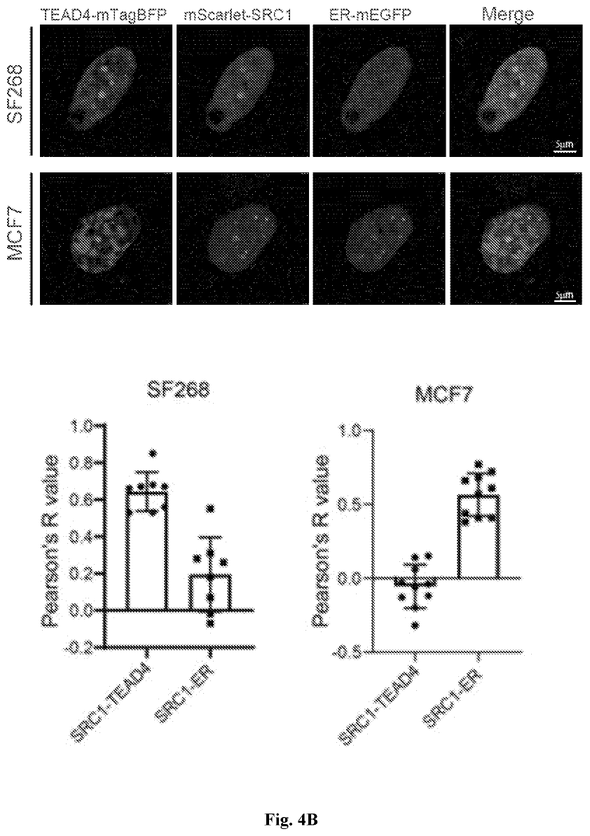

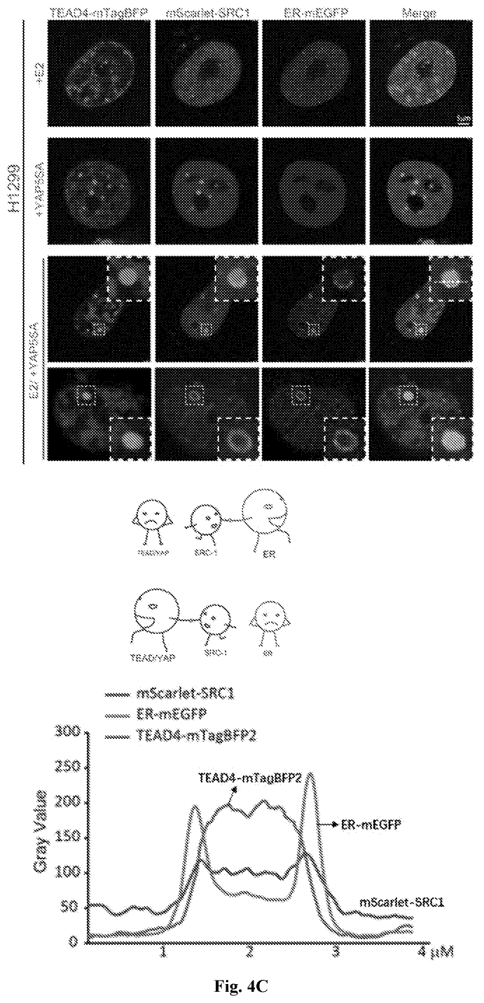

Microscopic images revealed that SRC-1 could form transcriptional condensates interplaying between the ERα signaling and Hippo pathway under different cell contexts (FIGS. 4A-C), highlighting the specificity of SRC-1 regulation on cell-specific transcriptional activation.

### Example 5

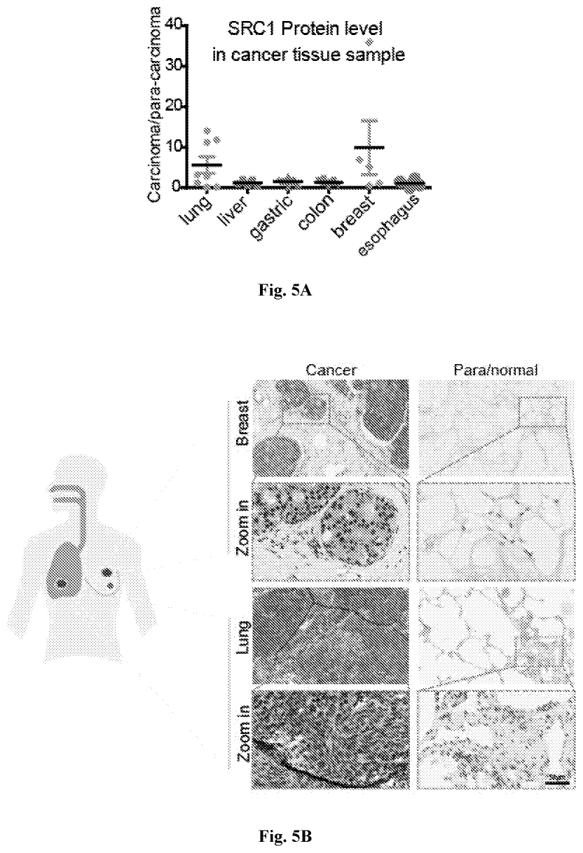

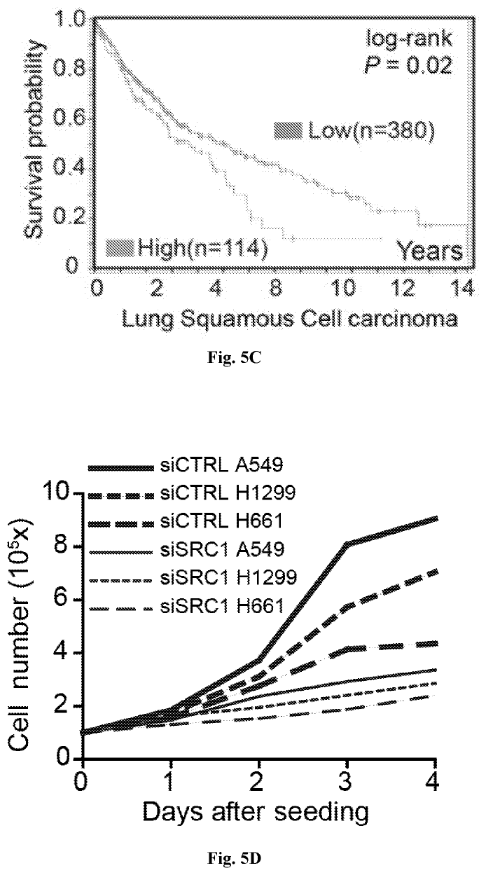

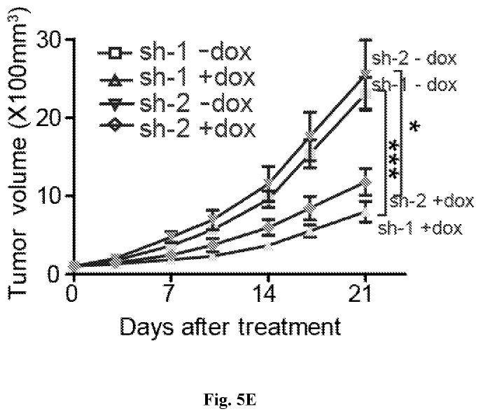

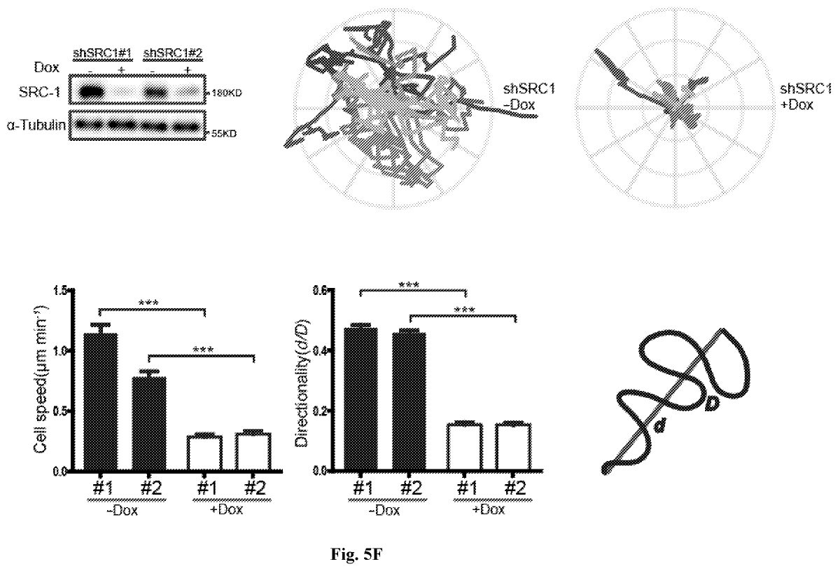

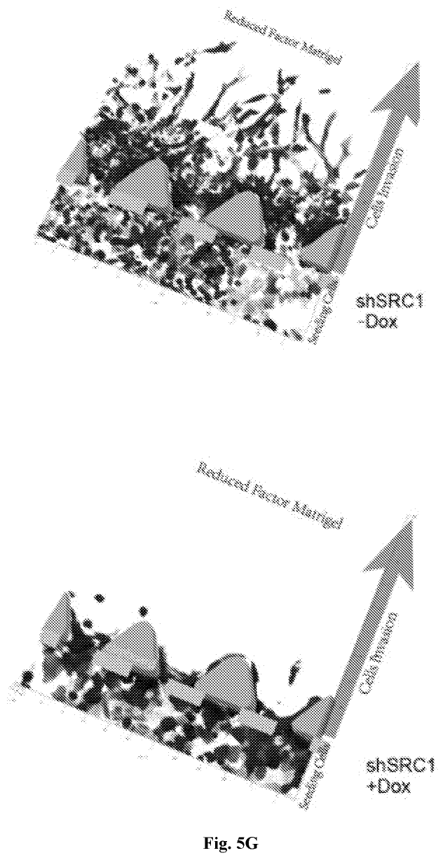

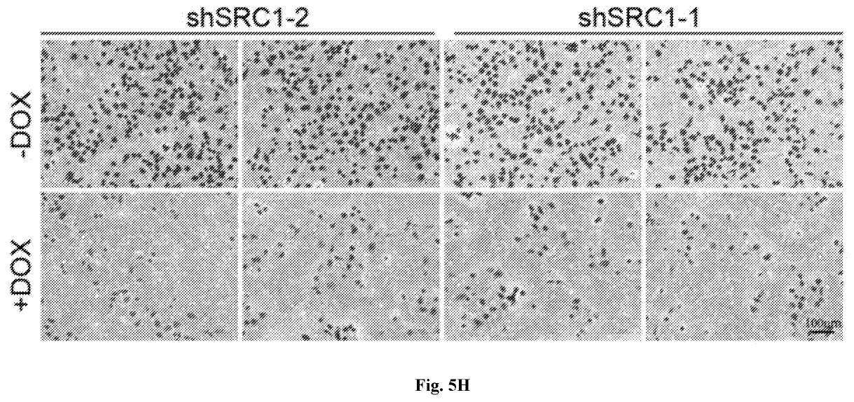

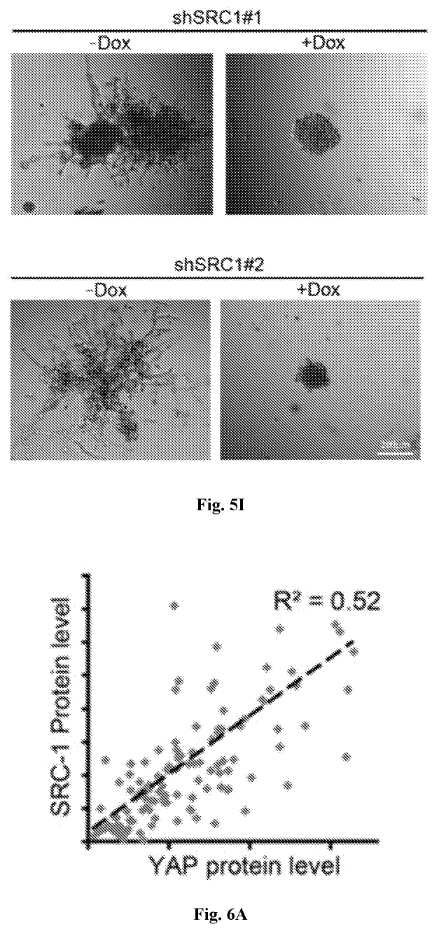

While SRC-1 has been associated with breast cancer previously (Redmond, A. M. et al. Clin Cancer Res. 15, 2098-2106 (2009)), elevated expression of SRC-1 was detected in NSCLCs which correlated with malignant features and poor prognosis (FIGS. 5A-C). A series of functional assays (FIGS. 5D-I) conducted using SRC-1 knockdown H1299 cells revealed that SRC-1 is essential for lung cancer proliferation, migration and invasion. These results support the noncanonical but critical oncogenic function of SRC-1 in NSCLCs.

### Example 6

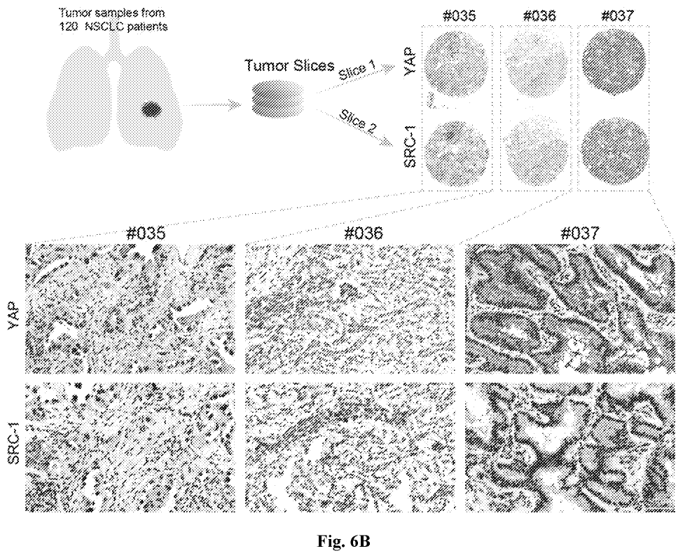

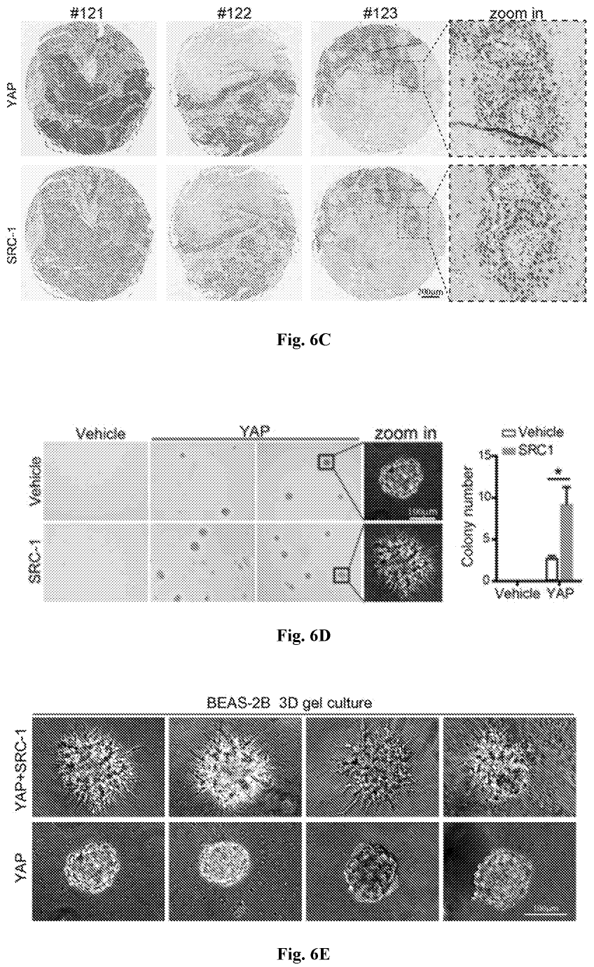

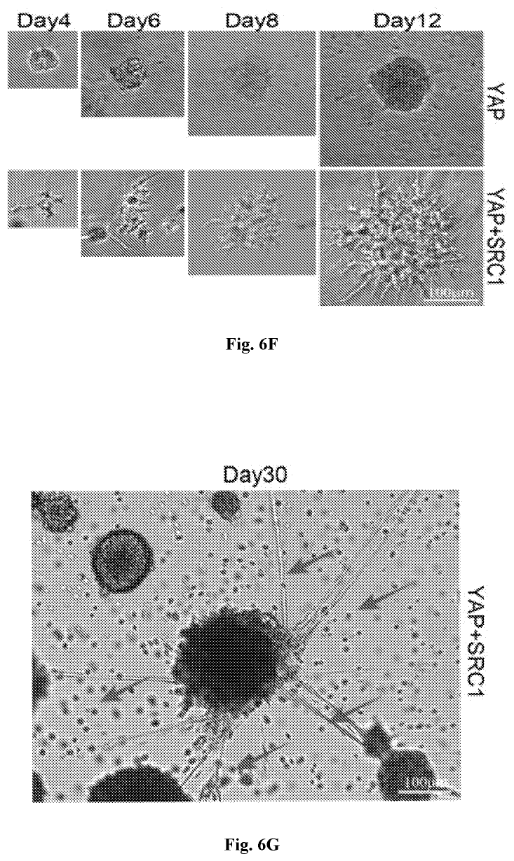

Analyzing SRC-1 and YAP expression in 120 NSCLCs samples by immunohistochemistry (IHC) found that SRC-1 and YAP were co-upregulated, with a strong correlation between the protein levels of SRC-1 and YAP (R²=0.52) (FIGS. 6A and 6B). Importantly, SRC-1 and YAP exhibited similar distribution pattern (FIG. 6C). To explore whether SRC-1 and YAP cooperate in driving tumorigenesis, normal human lung bronchial epithelium cells (BEAS-2B) was transformed with YAP and/or SRC-1. Microscopic observations revealed that both the number and extent of colony formation were more pronounced in BEAS-2B cells co-transfected with SRC-1 and YAP compared to that with YAP alone, whereas no colonies were formed with SRC-1 alone (FIGS. 6D-G). These results demonstrated that SRC-1 facilitated YAP to promote lung cancer progression.

### Example 7

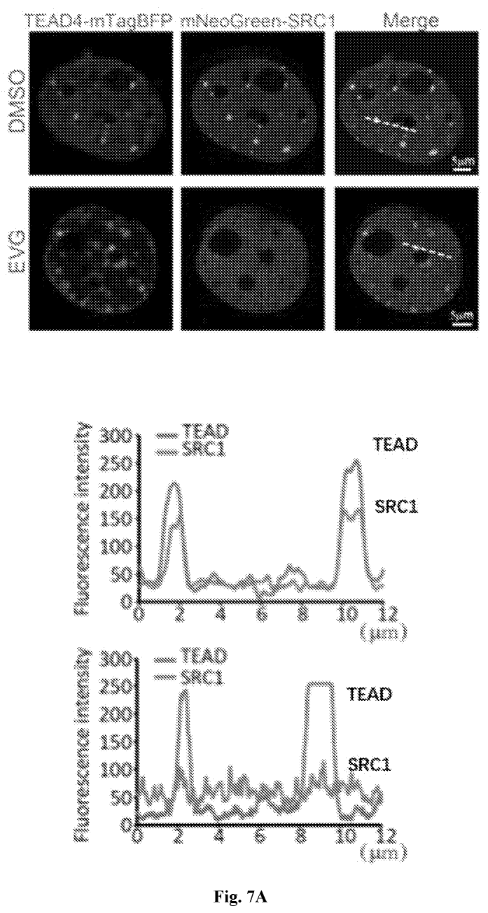

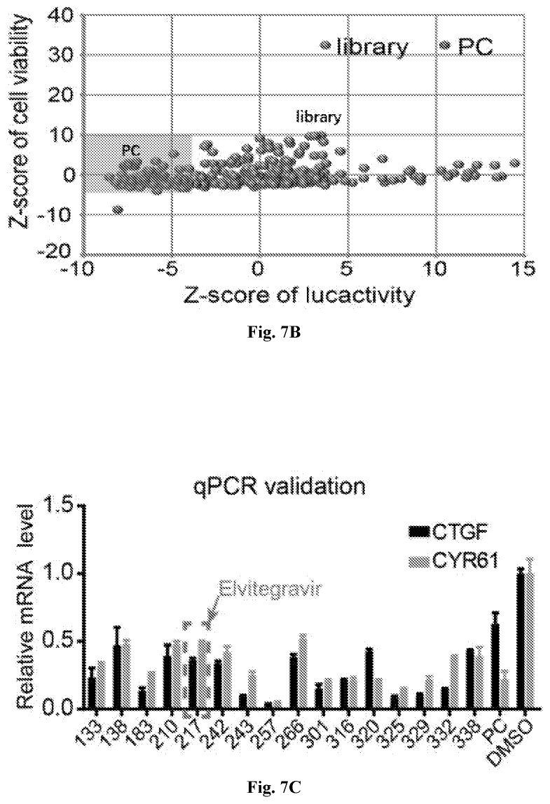

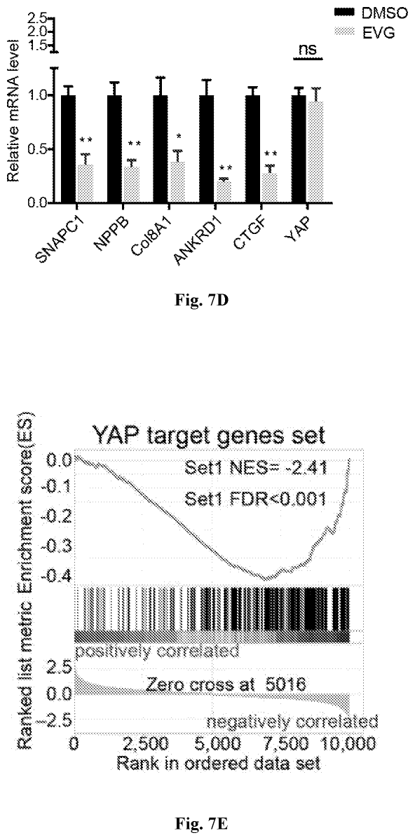

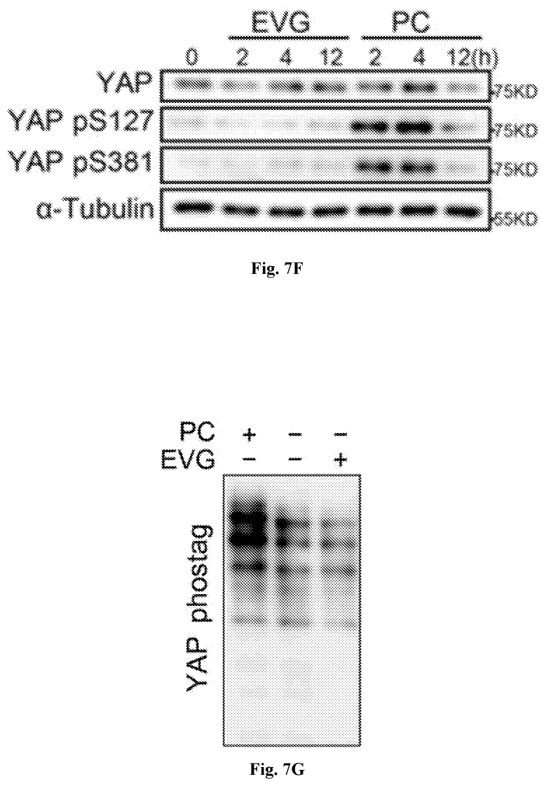

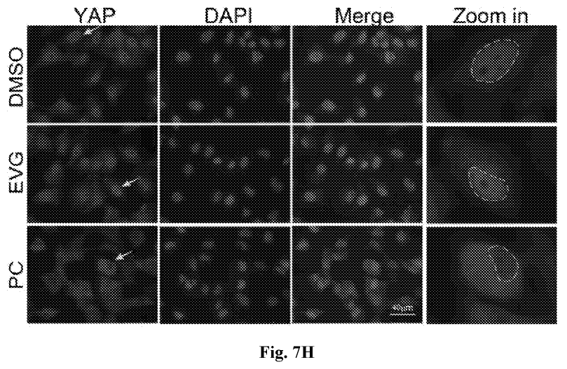

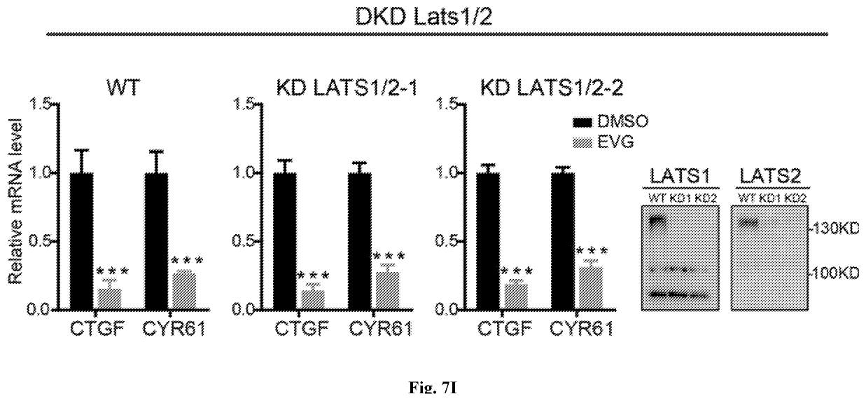

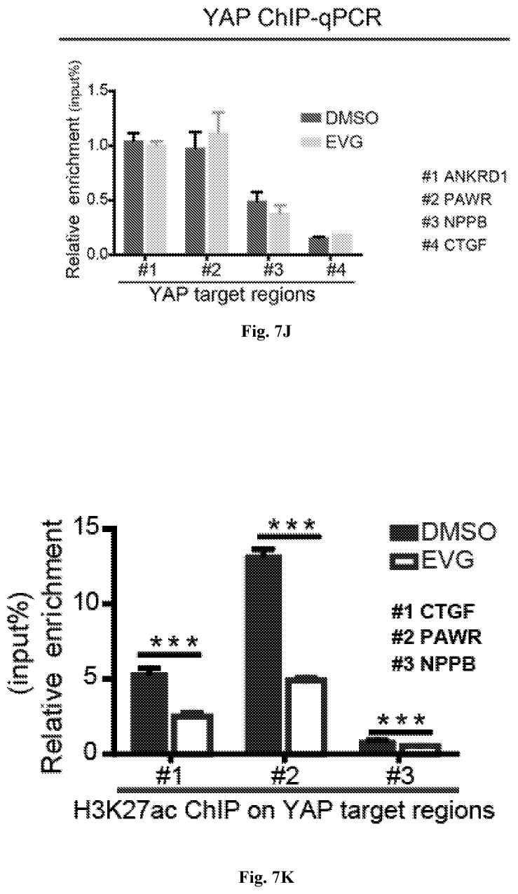

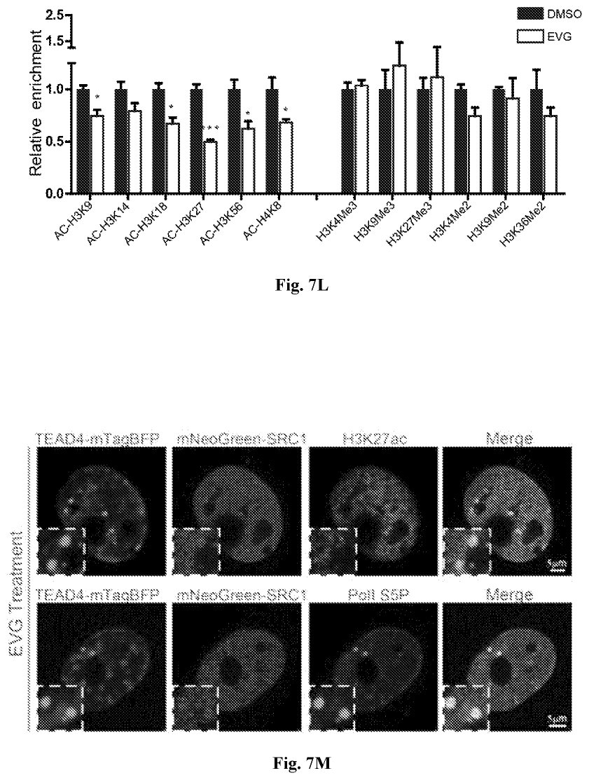

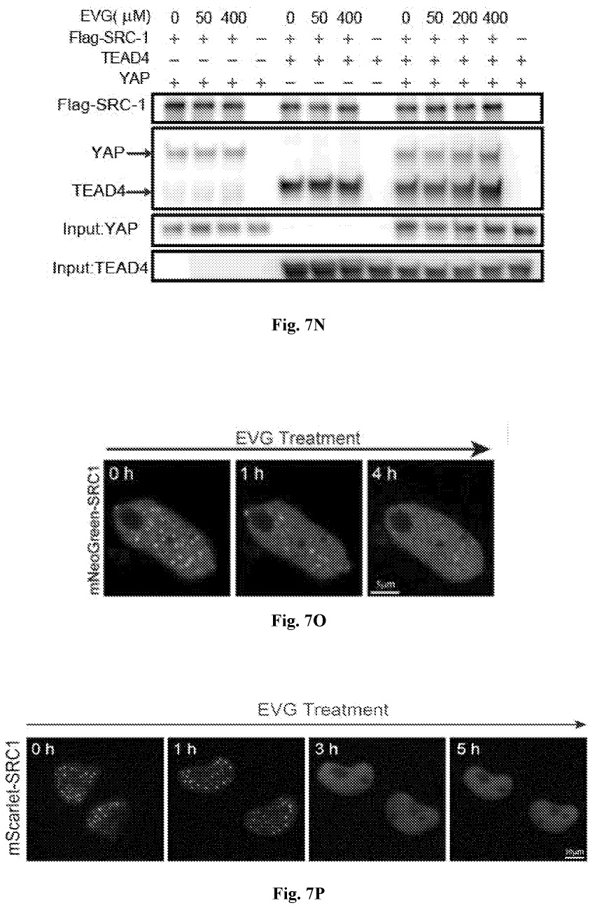

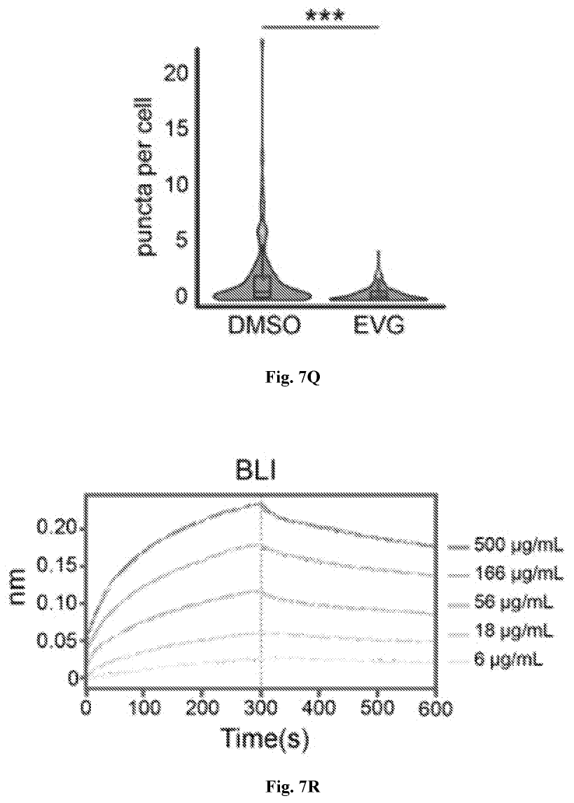

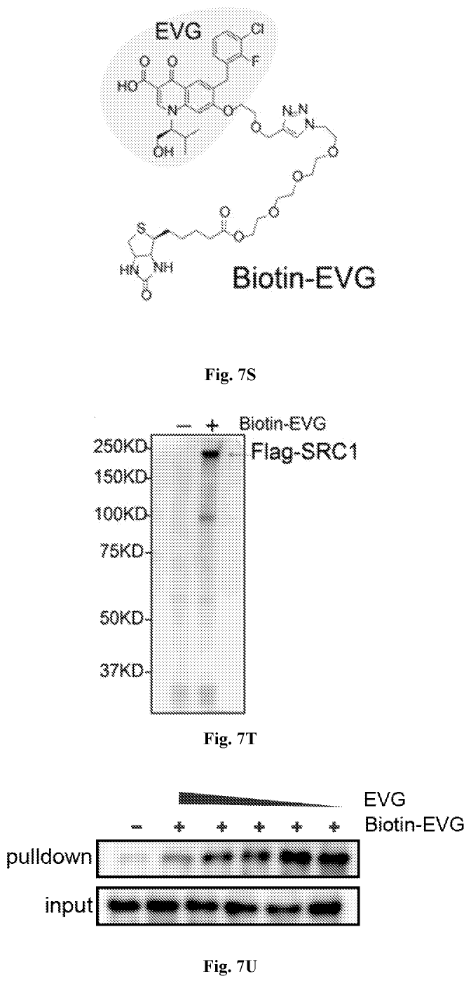

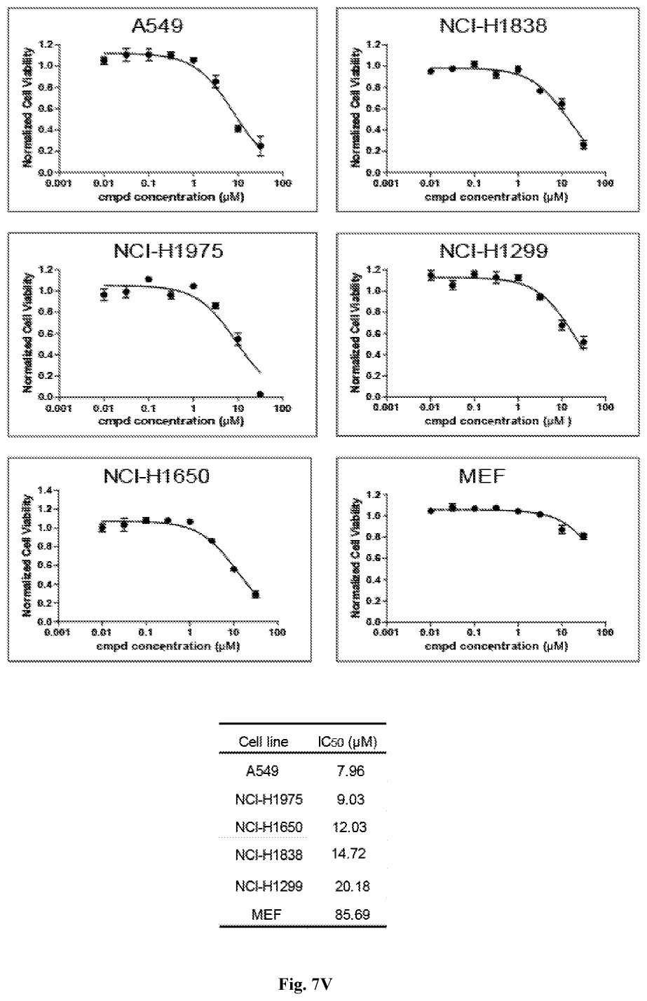

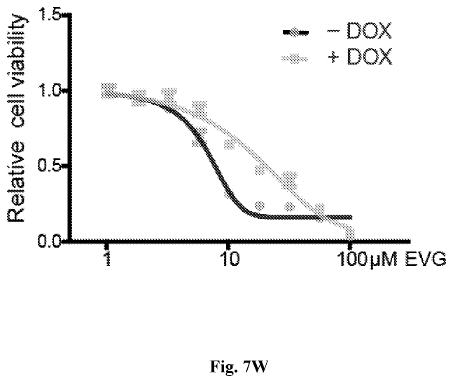

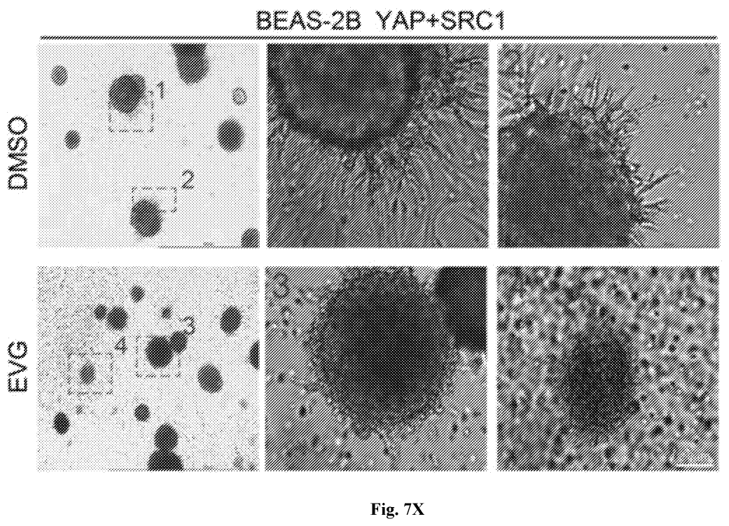

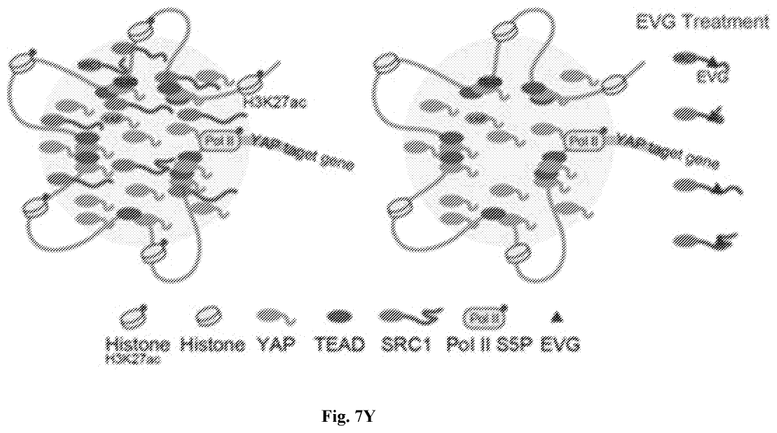

An anti-HIV drug elvitegravir (EVG) was identified from a YAP reporter cell-based screen in a library of FDA-approved drugs to suppress YAP activity (FIGS. 7B-D). The phase-separated SRC-1 condensates, but not the YAP/TEAD4 condensates, were selectively disrupted by the treatment of EVG (FIG. 7A). Global gene expression profiles verified that EVG downregulates YAP target genes (FIG. 7E). Particularly, EVG regulated YAP activity independent of canonical Hippo kinase cascade as revealed by the unaffected nuclear translocation and phosphorylation pattern in YAP (FIGS. 7F-I). After excluding the possibility that EVG may block the access of YAP to target genes (FIG. 7J), it is found that elvitegravir epigenetically regulates YAP by reducing H3K27ac mark levels at YAP target genes (FIGS. 7K and 7L). Importantly, microscopic images revealed that EVG inhibited the enrichment of H3K27ac at TEAD puncta, but exhibited no effect on RNA polymerase II phosphorylated at Ser 5 (CTD) (FIG. 7M). Next in vitro pulldown assays was conducted using recombinant proteins and it is found that EVG did not affect SRC-1's binding with YAP and TEAD even at concentrations up to 400 μM, indicating that the exclusion of SRC-1 from YAP/TEAD condensates was not due to interrupted binding with YAP or TEAD (FIG. 7N). Indeed, EVG suppressed the nuclear SRC-1 puncta formation in H1299 cells expressing either mNeoGreen or mScarlet labeled SRC-1 (FIGS. 7O and 7P). High content image results corroborated the inhibitory effects of EVG on SRC-1 phase separation (FIG. 7Q). These indicate that EVG directly disrupts the LLPS of SRC-1. Biophysical studies confirmed that EVG directly binds to SRC-1(FIG. 7R). Further competition pulldown and thermal shift assays (FIGS. 7S-U) indicated the binding was both direct and specific. It is found that EVG effectively inhibited the proliferation of lung cancer cell lines and inducible knockdown of SRC-1 rendered A549 cells partially resistant to elvitegravir's anti-proliferative effects (FIGS. 7V and 7W), suggesting that such effects are SRC-1-dependent. Moreover, treatment of SRC-1 and YAP co-expressed BEAS-2B cell colonies with EVG dramatically inhibited the migratory activity at the outer colony border (FIG. 7X). Taken together, EVG antagonized YAP oncogenic transcription activity by disturbing SRC-1 LLPS in SRC-1/YAP/TEAD condensates (FIG. 7Y).

### Examples 8: Pull-Down Assay

Characterization of Tested Compounds Binding with SRC-1 in Cellular Pull Down Assay:

Methods: Cells (SF268 cells and HEK293FT cells were transfected with expression vector of Flag-SRC-1) were harvested and lysed in lysis buffer (20 mM TrisHCl 7.5, 150 mM NaCl, 0.5% NP40, 5% glycerin, 1% proteinase Cocktail), and then incubated with 20 M compound with biotin tag or DMSO for 40 min at room temperature. Then incubated with 100 μL streptavidin-agarose (20359, Thermo Fisher Scientific) for 30 min at RT. The beads were washed 4 times with lysis buffer (4×583 1.5 min). And the precipitates were analyzed by western blot with anti-flag antibody.

Characterization of Tested Compounds Binding with SRC-1 In Vitro

1 μg of purified recombinant protein Flag-SRC-1 was incubated with 2 M biotin tagged compounds or DMSO as control in the presence of increasing concentrations of respective compounds without biotin tag (20, 40, 100, 588 200 μM) for 30 min (RT) and was further incubated with streptavidin-agarose (20359, Thermo Fisher Scientific) for 20 min (RT). Washing beads with PBS (3×1.5 min). The precipitates were analyzed by western blot with anti-flag antibody.

### Examples 9: Proliferation Assay

Characterization of Tested Compounds Effect on Cell Proliferation

Cells were cultured in 96-well ViewPlate by optimized density (200-2000 cells per well) and cultured in 100 μL medium as suggested by ATCC containing DMSO or compound. After 6 days [or other time period as indicated] culture, cell viability was measured using CellTiter-Glo® Luminescent Cell Viability Assay (Promega, G7572) and DMSO group was used as normalization to calculate half maximal inhibitory concentration (IC50) using Graphpad Prism software.

### Examples 10: RT-qPCR

Characterization of Tested Compounds Effect on SRC-1 Related Genes Expression:

Cells were treated with compounds for indicated time points, then total RNAs were extracted with TRIzol™ Reagent (15596018, Invitrogen) and reverse-transcripted using the High efficient cDNA synthesis master mix (FSQ-301,TOYOBO). Quantitative RT-PCR was performed with SYBR® Premix Ex Ta™ (RR420, Takara). Quantitative real-time PCR was carried out with QuantStudio™ 6 Flex Real-546 Time PCR Systems (Thermofisher Scientific). Qpcr Primers are listed Table 1.

TABLE 1
QPCR Primers

| | | | |
|---|---|---|---|
| ANKRD1 | Forward | TTTGGCAATTGTGGAGAAGT | SEQ ID |
| | | TA | NO: 1 |
| | Reverse | AAACATCCAGGTTTCCTCCA | SEQ ID |
| | | | NO: 2 |
| CYR61 | Forward | AAGAAACCCGGATTTGTGAG | SEQ ID |
| | | | NO: 3 |
| | Reverse | GCTGCATTTCTTGCCCTTT | SEQ ID |
| | | | NO: 4 |
| CTGF | Forward | CTCCTGCAGGCTAGAGAAGC | SEQ ID |
| | | | NO: 5 |
| | Reverse | GATGCACTTTTTGCCCTTCTT | SEQ ID |
| | | | NO: 6 |
| NPPB | Forward | GCTTTGGGAGGAAGATGGAC | SEQ ID |
| | | | NO: 7 |
| | Reverse | GCAGCCAGGACTTCCTCTTA | SEQ ID |
| | | | NO: 8 |
| YAP1 | Forward | GCAAATTCTCCAAAATGTCA | SEQ ID |
| | | GG | NO: 9 |
| | Reverse | CGGGAGAAGACACTGGATTT | SEQ ID |
| | | | NO: 10 |
| SNAPC1 | Forward | GAATGAAAGTTTGAGTGGAAC | SEQ ID |
| | | AGA | NO: 11 |
| | Reverse | CCAGGCTCTTTGTTCAGTGTT | SEQ ID |
| | | | NO: 12 |
| PAWR | Forward | CGTCCCCTACAAGCTCCTC | SEQ ID |
| | | | NO: 13 |
| | Reverse | GATGCCAGGAGACGACCTC | SEQ ID |
| | | | NO: 14 |
| Col8A1 | Forward | GAGGGCCAAGACGAAGACATC | SEQ ID |
| | | | NO: 15 |
| | Reverse | CAGATCACGTCATCGCACAAC | SEQ ID |
| | | | NO: 16 |
| BM2 | Forward | GAGGCTATCCAGCGTACTCCA | SEQ ID |
| | | | NO: 17 |
| | Reverse | CGGCAGGCATACTCATCTTTT | SEQ ID |
| | | | NO: 18 |
| GAPDH | Forward | GGAGCGAGATCCCTCCAAAAT | SEQ ID |
| | | | NO: 19 |
| | Reverse | GGCTGTTGTCATACTTCTCAT | SEQ ID |
| | | GG | NO: 20 |

### Examples 11: ChIP-qPCR

Characterization of Tested Compounds Effect on SRC-1 Related Chromatin Modifications:

Chromatin immunoprecipitation antibodies are listed in Supplementary 550 Supplementary Methods. Briefly, cells were crosslinked with 1% formaldehyde (Sigma) in culture medium for 10 min at room temperature, and chromatin from lysed nuclei was sheared to 200-1000 bp fragments using a Bioruptor® Pico (Diagenode SA). 50 g of sheared chromatin and 2 g of antibody were mixed. Antibody/antigen complexes were recovered with ProteinG-Dynabeads (Invitrogen) for 1.5 h at 4° C. Quantitative real-time PCR was carried out with QuantStudio™ 6 Flex Real-Time PCR Systems (Thermofisher Scientific). The amount of immunoprecipitated DNA in each sample was determined as the fraction of the input, and normalized to the IgG control.

TABLE 2
QPCR Primers

| | | | |
|---|---|---|---|
| Region#1 | Forward | ATGGCCTGCCACTTTGTTAC | SEQ ID |
| | primer | | NO: 21 |
| | Reverse | TTTTCAGAACTGGGGTCTGG | SEQ ID |
| | primer | | NO:22 |
| Region#2 | Forward | CAGCATTCCTGTCATTCCCT | SEQ ID |
| | primer | | NO: 23 |
| | Reverse | CAGGCTTCTTTTCTTGCACC | SEQ ID |
| | primer | | NO: 24 |
| Region#3 | Forward | TCTGGAATGCTGACCCTTCT | SEQ ID |
| | primer | | NO: 25 |
| | Reverse | CTTGGGTGACTTCGTCATCA | SEQ ID |
| | primer | | NO: 26 |
| #Ctrl | Forward | ACCAACACTCTTCCCTCAGC | SEQ ID |
| | primer | | NO: 27 |
| | Reverse | TTATTTTGGTTCAGGTGGTTGA | SEQ ID |
| | primer | | NO:28 |

### Examples 12: Live Cell Imaging for SRC-1 Condensates

Cells were grown on 24-well glass bottom plate (Cellvis, P24-1.5H-N) and images were taken with the Leica TCS SP8 confocal microscopy system using a 100× oil objective (NA=1.4). Cells were imaged on a heated stage (37° C.) and supplemented with warmed (37° C.) humidified air.

For compounds treatment assay, SF268 cells were co-transfected with YAP^{5SA}, TEAD4-mTagBFP2 and mNeoGreen-SRC1I. YAP variant YAP^{5SA} which is insensitive to the upstream Hippo pathway, sequestered YAP in nucleus. To circumvent the limitations brought by red fluorescent proteins, TEAD condensates which have been reported to depend on YAP, were used to characterize YAP/TEAD transcriptional condensates. Six hours after transfection, SF268 cells were seeded on 24-well glass bottom plate (Cellvis, P24-1.5H-N) and 20 μM compounds were added two hours later. The final concentration of DMSO was 0.1%. Cells were imaged every 30 mins after EVG or other compounds were added.

Data was analyzed by images collected from 100 representative fields in each group. The spots quantification was performed based on the number and intensity of the spots through green (mNeoGreen-SRC1) and blue (TEAD4-mTagBFP2) channel. Fluorescent images were processed and assembled into figures using LAS X (Leica) and Fiji. GraphPad Prism is used to plot and analyze the compound treatment results.

Live-cell images showing the distribution of TEAD4-mTagBFP and mNeoGreen-SRC1 in nucleus of H1299 cells co-transfected with YAP5SA plasmids treated w/o 20 μM EVG. Quantification of fluorescence intensity of TEAD4-mTagBFP and mNeoGreen-SRC1 along the line indicated in the merged image was shown on the right. TEAD condensates which is reported to depend on YAP were used to characterize YAP/TEAD transcriptional condensates. SRC-1 co-occupied with YAP/TEAD transcription condensates. After EVG treatment, SRC1 phase separation was disrupted while the YAP/TEAD condensates remained intact. Scale bar, 5 μm.

1. A method of modulating transcription of one or more genes in a cell or in a subject, comprising modulating a transcriptional SRC-1 condensate comprising at least SRC-1, wherein the transcriptional SRC-1 condensate regulates transcription of the one or more genes.

2. The method of claim 1, wherein the transcriptional SRC-1 condensate further comprises a first component capable of interacting with SRC-1.

3. The method of claim 2, wherein the first component comprises Yes-associated protein (YAP), Estrogen receptor (ER), androgen receptor (AR), vitamin D receptor (VDR) and AP-1.

4. The method of claim 2, wherein the transcriptional SRC-1 condensate further comprises a second component interacting with the first component.

5. The method of claim 4, wherein the second component comprises a TEA-domain transcription factor.

6. The method of claim 5, wherein the TEA-domain transcription factor comprises TEAD1, TEAD2, TEAD3, TEAD4 or any combination thereof.

7. The method of claim any one of the preceding claims, wherein the transcriptional SRC-1 condensate further comprises a RNA polymerase.

8. The method of claim any one of the preceding claims, wherein the transcriptional SRC-1 condensate is modulated by modulating or reducing formation, composition, stability, and/or activity of the transcriptional SRC-1 condensate.

9. The method of claim 8, wherein the transcriptional SRC-1 condensate is modulated by contacting with a SRC-1 condensate inhibitor.

10. The method of claim 9, wherein the SRC-1 condensate inhibitor is capable of
a) reducing formation or stability of a SRC-1 condensate,
b) reducing or eliminating interactions between SRC-1 and one or more components in the transcriptional condensate, optionally in a SRC-1 selective manner,
c) reducing or eliminating binding of SRC-1 to one or more components in the transcriptional condensate, optionally in a SRC-1 selective manner, or
d) sequestering SRC-1 outside the transcriptional condensate.

11. The method of claim 10, wherein the one or more components comprises YAP, Estrogen receptor (ER), androgen receptor (AR), vitamin D receptor(VDR) and AP-1.

12. The method of claim 10 or 11, wherein the SRC-1 condensate inhibitor interacts with an intrinsic disorder domain of SRC-1.

13. The method of claim 10 or 11, wherein the SRC-1 condensate inhibitor binds to a non-intrinsic disordered domains (non-IDD) region of SRC-1, and optionally allosterically induces a conformational change in the IDD.

14. The method of any of claims 10-11, wherein the SRC-1 condensate inhibitor sequesters SRC-1 outside the transcriptional condensate, optionally without significantly dissolving the transcriptional condensate without SRC-1.

15. The method of any of claims 10-14, wherein the SRC-1 condensate inhibitor comprises a peptide, nucleic acid, or small molecule.

16. The method of any of claims 10-15, wherein the SRC-1 condensate inhibitor decreases level of transcriptional SRC-1 condensate by at least 30% (e.g. at least 40%, 50%, 60%, or 70%) at a concentration of no more than 20 uM.

17. The method of any of claims 10-16, wherein the SRC-1 condensate inhibitor comprises elvitegravir (EVG), or competes with EVG for binding to SRC-1, or induces a conformational change in SRC-1 at least comparable to that induced by EVG.

18. The method of any of claims 10-16, wherein the SRC-1 condensate inhibitor has comparable activity to EVG or higher activity than EVG in decreasing level of transcriptional SRC-1 condensate.

19. The method of any of the preceding claims, wherein the transcription of the one or more genes is associated with an oncogenic signaling pathway.

20. The method of any of the preceding claims, wherein the one or more genes comprise one or more oncogenes.

21. The method of any of the preceding claims, wherein the one or more genes comprise one or more YAP target genes.

22. The method of claim 21, wherein the one or more YAP target genes are selected from the group consisting of ANKRD1, CTGF, and CYR61.

23. The method of claim 22, wherein the cell or the subject has an elevated expression level of SRC-1 relative to a reference level.

24. A method of modulating transcription of one or more YAP target genes in a cell or in a subject, comprising modulating SRC-1 by a SRC-1 inhibitor.

25. The method of claim 24, wherein the SRC-1 inhibitor is capable of reducing expression level or reducing activity of SRC-1, or is a SRC-1 condensate inhibitor.

26. The method of claim 24 or 25, wherein SRC-1 inhibitor comprises a peptide, nucleic acid, or small molecule.

27. The method of claim 26, wherein the nucleic acid comprises an oligonucleotide specifically hybridizable to SRC-1 mRNA, or a polynucleotide encoding the oligonucleotide, and/or wherein the peptide comprises a SRC-1 mimetic.

28. The method of claim 27, wherein the oligonucleotide comprises siRNA, shRNA, miRNA, or antisense oligonucleotide.

29. The method of any of claims 24-28, the one or more YAP target genes are selected from the group consisting of ANKRD1, CTGF, and CYR61.

30. The method of any of claims 24-29, the cell or the subject has an elevated expression level of SRC-1 relative to a reference level.

31. A method of treating a SRC-1 condensate associated disease or condition, or YAP-associated disease or condition in a subject, comprising administering to the subject a pharmaceutically effective amount of a SRC-1 inhibitor.

32. The method of claim 31, wherein the disease or condition is characterized in an elevated expression level of SRC-1 relative to a reference level.

33. The method of claim 31, wherein the disease or condition is associated with aberrant expression of an oncogene.

34. The method of claim 31, wherein the disease or condition is associated with aberrant expression of a YAP target gene.

35. The method of claim 31, wherein the disease or condition is associated with aberrant YAP transcription activity.

36. The method of any of claims 31-35, wherein the disease or condition is cancer.

37. The method of claim 36, wherein the cancer is metastatic.

38. The method of claim 36 or 37, wherein the cancer is breast cancer, lung cancer, adrenal cancer, lymphoepithelial neoplasia, adenoid cell carcinoma, lymphoma, acoustic neuroma, acute lymphocytic leukemia, acral lentiginous melanoma, acute myeloid leukemia, acrospiroma, chronic lymphocytic leukemia, acute eosinophilic leukemia, liver cancer, acute erythrocyte leukemia, small cell lung cancer, acute lymphocytic leukemia, non-small cell lung cancer, acute megakaryoblastic leukemia, MALT lymphoma, acute monocytic leukemia, malignant fibrous histiocytoma, acute promyelocytic leukemia, malignant peripheral schwannomas, mantle cell lymphoma, adenocarcinoma, marginal zone B-cell lymphoma, malignant hippocampal tumor, adenoid cystic carcinoma, gland tumor, adenoma-like odontogenic tumor, mast cell leukemia, adenosquamous carcinoma, mediastinal germ cell tumor, adipose tissue tumor, breast medullary carcinoma, adrenocortical carcinoma, medullary thyroid carcinoma, adult T cell leukemia/lymphoma, Medulloblastoma, invasive NK cell leukemia, melanoma, AIDS-related lymphoma, meningioma, lung rhabdomyosarcoma, Merkel cell carcinoma, alveolar soft tissue sarcoma, mesothelioma, ameloblastoma, metastatic urothelial carcinoma, anaplastic large cell lymphoma, mixed Müllerian tumor, thyroid undifferentiated carcinoma, mucinous neoplasm, angioimmunoblastic T-cell lymphoma, multiple myeloma, angiomyolipoma, muscle tissue tumor, angiosarcoma, mycosis fungoides, astrocytoma, myxoid liposarcoma, atypical deformed rhabdoid tumor, myxoma, B-cell chronic lymphocytic leukemia, mucinous sarcoma, B-cell lymphoblastic leukemia, nasopharyngeal carcinoma, B-cell lymphoma, schwannomas, basal cell carcinoma, neuroblastoma, biliary tract cancer, neurofibromatosis, bladder cancer, neuroma, blastoma, nodular melanoma, bone cancer, eye cancer, Brenner tumor, oligodendroxoma, brown tumor, oligodendroglioma, Burkitt's lymphoma, eosinophilic breast cancer, brain cancer, optic nerve tumor cancer, oral cancer carcinoma in situ, osteosarcoma, carcinosarcoma, ovarian cancer, cartilage tumor, pulmonary sulcus tumor, papillary thyroid carcinoma, myeloma, paraganglioma, chondroma, pineal blastoma, chordoma, pineal cell tumor, choriocarcinoma, pituitary tumor, choroid plexus papilloma, pituitary adenoma, kidney clear cell sarcoma, pituitary tumor, craniopharyngioma, plasmacytoma, cutaneous T-cell lymphoma, multiple embryonic cell tumor, cervical cancer, precursor T lymphoblastic lymphoma, colorectal cancer, primary central nervous system lymphoma, Degos disease, primary effusion lymphoma, proliferative small round cell tumor, primary preformed peritoneal cancer, diffuse large B-cell lymphoma, prostate cancer, embryonic dysplasia of neuroepithelial neoplasia, pancreatic cancer, anaplastic cell tumor, pharyngeal carcinoma, embryonic carcinoma, peritoneal pseudomyxoma, endocrine gland tumor, renal cell carcinoma, enteropathy-associated T-cell lymphoma, endodermal sinus tumor, renal medullary carcinoma, retinoblastoma, esophageal cancer, rhabdomyosarcoma, endadelphos, rhabdomyosarcoma, fibroids, Richter's transformation, fibrosarcoma, rectal cancer, follicular lymphoma, sarcoma, follicular thyroid cancer, schwannoma, ganglion cell tumor, seminoma, gastrointestinal cancer, Sertoli cell tum, germ cell tumor, sex cord-gonadal stromal tumor, pregnancy choriocarcinoma, signet ring cell carcinoma, giant cell fibroblastoma, skin cancer, bone giant cell tumor of bone, small blue round cell tumor, glioma, small cell carcinoma, glioblastoma multiforme, soft tissue sarcoma, glioma, somatostatin tumor, glioma brain, soot wart, pancreatic high glucagonoma, spinal tumor, Gonadoblastoma, spleen marginal lymphoma, granulosa cell tumor, squamous cell carcinoma, estrogen tumor, synovial sarcoma, gallbladder cancer, Sezary disease, gastric cancer, small intestine cancer, hairy cell leukemia, squamous cell carcinoma, hemangioblastoma, gastric cancer, head and neck cancer, T-cell lymphoma, vascular epithelioma, testicular cancer, hematological malignancies, sarcoma, hepatoblastoma, thyroid cancer, hepatosplenic T-cell lymphoma, transitional cell carcinoma, Hodgkin's lymphoma, laryngeal cancer, non-Hodgkin's lymphoma, urachal cancer, invasive lobular carcinoma, genitourinary cancer, intestinal cancer, urothelial carcinoma, renal cancer, uveal melanoma, laryngeal cancer, uterine cancer, malignant freckle-like sputum, verrucous carcinoma, lethal midline granuloma, visual pathway glioma, leukemia, vulvar cancer, testicular stromal tumor, vaginal cancer, liposarcoma, Waldenstrom's macroglobulinemia Disease, adenolymphoma, lymphangioma, nephroblastoma, lymphangisarcoma.

39. The method of claim 36 or 37, wherein the cancer is breast cancer, lung cancer (optionally non-small cell lung cancer), uveal melanoma, liver cancer, head neck cancer and squamous carcinoma, mesothelioma, or malignant pleural mesothelioma.

40. The method of any of claims 31-39, wherein the SRC-1 inhibitor is capable of reducing expression level or reducing biological activity of SRC-1.

41. The method of any of claims 31-40, wherein the SRC-1 inhibitor comprises a peptide, nucleic acid, or small molecule.

42. The method of claim 41, wherein the nucleic acid comprises an oligonucleotide specifically hybridizable to SRC-1 mRNA, or a polynucleotide encoding the oligonucleotide.

43. The method of claim 42, wherein the oligonucleotide comprises siRNA, shRNA, miRNA, or antisense oligonucleotide.

44. The method of claim 41, wherein the SRC-1 inhibitor is a SRC-1 mimetic. The method of any of claims 31-39, wherein the SRC-1 inhibitor comprises a SRC-1 condensate inhibitor.

45. The method of claim 44, wherein the SRC-1 condensate inhibitor is capable of:
a) reducing formation, composition, or stability of a SRC-1 condensate,
b) reducing or eliminating interactions between SRC-1 and one or more components in the transcriptional condensate, optionally in a SRC-1 selective manner,
c) reducing or eliminating binding of SRC-1 to one or more components in the transcriptional condensate, optionally in a SRC-1 selective manner, or
d) sequestering SRC-1 outside the transcriptional condensate.

46. The method of claim 45, wherein the one or more components comprises YAP.

47. The method of any of claims 44-46, wherein the SRC-1 condensate inhibitor interacts with an intrinsic disorder domain of SRC-1.

48. The method of any of claims 44-46, wherein the SRC-1 condensate inhibitor binds to a non-IDD region of SRC-1, and optionally allosterically induces a conformational change in the IDD.

49. The method of any of claims 44-48, wherein the SRC-1 condensate inhibitor sequesters SRC-1 outside the transcriptional condensate, optionally without significantly dissolving the transcriptional condensate without SRC-1.

50. The method of any of claims 44-49, wherein the SRC-1 condensate inhibitor decreases level of transcriptional SRC-1 condensate by at least 30% (e.g. at least 40%, 50%, 60%, or 70%) at a concentration of no more than 20 uM.

51. The method of any of claims 44-50, wherein the SRC-1 condensate inhibitor comprises elvitegravir (EVG), or competes with EVG for binding to SRC-1, or induces a conformational change in SRC-1 at least comparable to that induced by EVG.

52. The method of any of claims 44-51, wherein the SRC-1 condensate inhibitor has comparable activity to EVG or higher activity than EVG in decreasing level of transcriptional SRC-1 condensate.

53. A method of screening for an agent that modulates a SRC-1 condensate, comprising:
a. providing the SRC-1 condensate and assessing one or more physical properties or one or more biological effects of the condensate,
b. contacting the SRC-1 condensate with a test agent, and
c. assessing whether the test agent causes a change in the one or more physical properties or one or more biological effects of the SRC-1 condensate.

54. The method of claim 53, wherein the test agent is identified as modulating the condensate if it causes a change in the one or more physical properties or one or more biological effects of the SRC-1 condensate.

55. A method of identifying an agent that modulates formation of a SRC-1 condensate, comprising:
a. providing components capable of forming the SRC-1 condensate;
b. contacting the components with a test agent under the condition suitable for formation of the SRC-1 condensate, and
c. assessing whether presence of the test agent affects formation of the SRC-1 condensate or one or more biological effects of the SRC-1 condensate.

56. The method of claim 55, wherein the test agent is identified as modulating the formation of the condensate if it affects formation of the condensate or affects the one or more biological effects of the SRC-1 condensate.

57. The method of any of claims 53-56, wherein the SRC-1 condensate is an isolated synthetic condensate, or is in the form of an isolated cellular composition comprising the SRC-1 condensate.

58. The method of any of claims 53-56, wherein the SRC-1 condensate is inside a cell or inside nucleus.

59. The method of any of claims 53-58, wherein the SRC-1 condensate is a transcriptional condensate.

60. The method of any of claims 53-59, wherein the one or more biological effects of the transcriptional condensate is assessed based on expression of a target gene in a condensate-dependent manner.

61. The method of claim 60, wherein the target gene is a reporter gene.

62. The method of any one of claims 60-61, wherein the target gene is a YAP regulated gene.

63. The method of any one of claims 59-62 wherein the transcriptional SRC-1 condensate further comprises a first component capable of interacting with SRC-1.

64. The method of claim 63, wherein the first component comprises Yes-associated protein (YAP), Estrogen receptor (ER), androgen receptor(AR), vitamin D receptor(VDR) and AP-1.

65. The method of claim 63 or 64, wherein the transcriptional condensate further comprises a second component interacting with the first component.

66. The method of claim 65, wherein the second component comprises a TEA-domain transcription factor.

67. The method of claim 66, wherein the TEA-domain transcription factor comprises TEAD1, TEAD2, TEAD3, TEAD4 or any combination thereof.

68. The method of claim any one of claims 59-67, wherein the transcriptional condensate further comprises a RNA polymerase.

69. A synthetic SRC-1 condensate comprising at least SRC-1 or a fragment thereof comprising an intrinsic disorder domain of SRC-1.

70. The synthetic SRC-1 condensate of claim 69, wherein the fragment is fused with an inducible oligomerization domain.

71. An in vitro screening system comprising SRC-1 or a fragment thereof comprising an intrinsic disorder domain of SRC-1, and a detectable label, wherein the SRC-1 or the fragment thereof is capable of forming SRC-1 condensate.

72. The in vitro screening system of claim 72, wherein the detectable label is attached to the SRC-1 or the fragment thereof.

73. The in vitro screening system of claim 73, wherein the detectable label comprises a fluorophore, a radioisotope, a colorimetric substrate, or an antigenic epitope.

74. The in vitro screening system of claim 73, wherein the in vitro screening system further comprises further comprises a first component capable of interacting with SRC-1.

75. The in vitro screening system of claim 74, wherein the first component comprises Yes-associated protein (YAP), Estrogen receptor (ER), androgen receptor(AR), vitamin D receptor(VDR) and AP-1.

76. The in vitro screening system of any one of claims 74-75, further comprising a second component interacting with the first component.

77. The in vitro screening system of claim 76, wherein the second component comprises a TEA-domain transcription factor.

78. The in vitro screening system of claim 77, wherein the TEA-domain transcription factor comprises TEAD1, TEAD2, TEAD3, TEAD4 or any combination thereof.

79. The in vitro screening system of claim any one of claims 71-78, further comprising a RNA polymerase.

80. The in vitro screening system of any one of claims 71-79, further comprising a cell lysate or a nuclear lysate.

81. A modified host cell expressing SRC-1 or a fragment thereof comprising an intrinsic disorder domain, wherein the SRC-1 or the fragment is capable of forming SRC-1 condensate, and wherein the host cell further comprises a detectable label that allows for detection of the SRC-1 condensate.

82. The modified host cell of claim 81, wherein the detectable label is attached to the SRC-1 or the fragment thereof.

83. The modified host cell of claim 82, wherein the detectable label comprises a fluorophore, a radioisotope, a colorimetric substrate, or an antigenic epitope.

84. The modified host cell of any of claims 81-83, wherein the host cell is a tumor cell.

85. A modified host cell expressing: a) SRC-1 or a fragment thereof comprising an intrinsic disorder domain thereof, and b) YAP or a functional equivalent thereof, wherein the host cell comprises a YAP-responsive reporter construct.

86. The modified host cell of claim 85, wherein the YAP-responsive reporter construct comprises a reporter gene operably linked to a promoter responsive to YAP activity.

87. A method of screening for an agent that inhibits SRC-1, comprising:
a. contacting a test agent with the modified host cell of claim 86 under a condition suitable for expression of the reporter gene;
b. assessing change in expression of the reporter gene in response to the test agent;
wherein the change in expression of the reporter gene indicates inhibition of SRC-1.


SEQUENCE LISTING (28 sequences)

SEQ ID NO: 1 - DNA, 22 bp, Artificial Sequence
  Synthetic
tttggcaattgtggagaagtta

SEQ ID NO: 2 - DNA, 20 bp, Artificial Sequence
  Synthetic
aaacatccaggtttcctcca

SEQ ID NO: 3 - DNA, 20 bp, Artificial Sequence
  Synthetic
aagaaacccggatttgtgag

SEQ ID NO: 4 - DNA, 19 bp, Artificial Sequence
  Synthetic
gctgcatttcttgcccttt

SEQ ID NO: 5 - DNA, 20 bp, Artificial Sequence
  Synthetic
ctcctgcaggctagagaagc

SEQ ID NO: 6 - DNA, 21 bp, Artificial Sequence
  Synthetic
gatgcactttttgcccttctt

SEQ ID NO: 7 - DNA, 20 bp, Artificial Sequence
  Synthetic
gctttgggaggaagatggac

SEQ ID NO: 8 - DNA, 20 bp, Artificial Sequence
  Synthetic
gcagccaggacttcctctta

SEQ ID NO: 9 - DNA, 22 bp, Artificial Sequence
  Synthetic
gcaaattctccaaaatgtcagg

SEQ ID NO: 10 - DNA, 20 bp, Artificial Sequence
  Synthetic
cgggagaagacactggattt

SEQ ID NO: 11 - DNA, 24 bp, Artificial Sequence
  Synthetic
gaatgaaagtttgagtggaacaga

SEQ ID NO: 12 - DNA, 21 bp, Artificial Sequence
  Synthetic
ccaggctctttgttcagtgtt

SEQ ID NO: 13 - DNA, 19 bp, Artificial Sequence
  Synthetic
cgtcccctacaagctcctc

SEQ ID NO: 14 - DNA, 19 bp, Artificial Sequence
  Synthetic
gatgccaggagacgacctc

SEQ ID NO: 15 - DNA, 21 bp, Artificial Sequence
  Synthetic
gagggccaagacgaagacatc

SEQ ID NO: 16 - DNA, 21 bp, Artificial Sequence
  Synthetic
cagatcacgtcatcgcacaac

SEQ ID NO: 17 - DNA, 21 bp, Artificial Sequence
  Synthetic
gaggctatccagcgtactcca

SEQ ID NO: 18 - DNA, 21 bp, Artificial Sequence
  Synthetic
cggcaggcatactcatctttt

SEQ ID NO: 19 - DNA, 21 bp, Artificial Sequence
  Synthetic
ggagcgagatccctccaaaat

SEQ ID NO: 20 - DNA, 23 bp, Artificial Sequence
  Synthetic
ggctgttgtcatacttctcatgg

SEQ ID NO: 21 - DNA, 20 bp, Artificial Sequence
  Synthetic
atggcctgccactttgttac

SEQ ID NO: 22 - DNA, 20 bp, Artificial Sequence
  Synthetic
ttttcagaactggggtctgg

SEQ ID NO: 23 - DNA, 20 bp, Artificial Sequence
  Synthetic
cagcattcctgtcattccct

SEQ ID NO: 24 - DNA, 20 bp, Artificial Sequence
  Synthetic
caggcttcttttcttgcacc

SEQ ID NO: 25 - DNA, 20 bp, Artificial Sequence
  Synthetic
tctggaatgctgacccttct

SEQ ID NO: 26 - DNA, 20 bp, Artificial Sequence
  Synthetic
cttgggtgacttcgtcatca

SEQ ID NO: 27 - DNA, 20 bp, Artificial Sequence
  Synthetic
accaacactcttccctcagc

SEQ ID NO: 28 - DNA, 22 bp, Artificial Sequence
  Synthetic
ttattttggttcaggtggttga